(19) United States
(12) Patent Application Publication
(10) Pub. No.: US 2008/0221193 A1
(43) Pub. Date: Sep. 11, 2008

(54) 3-AMINO CHROMANE DERIVATIVES

(75) Inventors: Minsheng Zhang, Warren, NJ (US); Charles J. Stanton, Jersey City, NJ (US); Nicole T. Hatzenbuhler, Bridgewater, NJ (US); Boyd Lynn Harrison, Princeton Junction, NJ (US)

Correspondence Address: WilmerHale/Wyeth, 60 STATE STREET, BOSTON, MA, 02109, US

(73) Assignee: Wyeth, Madison, NJ (US)

(21) Appl. No.: 11/963,126

(22) Filed: Dec. 21, 2007

Related U.S. Application Data
(60) Provisional application No. 60/876,898, filed on Dec. 22, 2006.

(51) Int. Cl. A61K 31/404; C07D 405/12; C07K 14/705; A61P 25/00; A61P 25/16; A61P 25/28; A61P 25/30

(52) U.S. Cl. 514/414; 548/454; 530/350

(57) ABSTRACT

The present invention relates to 3-amino chromane derivatives; to compositions containing such compounds; to methods of synthesizing such compounds; and to methods of using such compounds and compositions containing such compounds in the treatment of serotonin disorders, such as depression and anxiety.

### CROSS REFERENCE TO RELATED APPLICATIONS

This application claims the benefit of priority under 35 U.S.C. § 119(e) to U.S. Provisional Patent Application No. 60/876,898 filed on Dec. 22, 2006 and is hereby incorporated by reference in its entirety.

Throughout this application, various publications are referenced. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure, as it appears in the U.S. Patent and Trademark Office patent file or records, but otherwise reserves any and all copyright rights whatsoever.

### FIELD OF THE INVENTION

Novel 3-amino chormane derivative compounds are disclosed, as well as their activity as serotonin modulators and as 5-HT_{1A} receptor agonists or antagonists, and processes for preparing them, methods of using them and to pharmaceutical compositions containing them.

### BACKGROUND

Depression is a serious health problem affecting a significant proportion of the population. Selective serotonin reuptake inhibitors (SSRIs) have produced success in treating depression and related illnesses and have become among the most prescribed drugs. They nonetheless have a slow onset of action, often taking several weeks to produce their full therapeutic effect. They also exhibit undesirable side effects, such as sexual dysfunction. Furthermore, SSRIs are effective in less than two-thirds of patients.

It has been proposed that a 5-HT_{1A} antagonist may improve the efficacy of the serotonin reuptake mechanism (Perez, V., et al, The Lancet, 349:1594-1597 (1997)). Such a combination therapy would be expected to speed up the effect of the serotonin reuptake inhibitor.

Certain 3-amino chormane and 2-amino teralin derivatives have been reported to possess pharmaceutical activity. (See, for example, WO 2005/012291, which is incorporated by reference in its entirety). In particular, these compounds are reported to have the ability to block the reuptake of serotonin, as well as having an affinity for 5-HT_{1A} serotonin receptors. Other chormane derivatives have also been reported to possess pharmaceutical activity relating to the 5-HT receptors and/or the central nervous system. (See, for example, U.S. Pat. No. 5,420,151; WO 95/11891; and WO 90/12795).

### SUMMARY

The 3-amino chormane derivatives described herein are useful in the treatment and/or prevention of 5HT_{1A}- and/or serotonin-related disorders. The compounds described herein have the ability to bind 5-HT_{1A} receptors, act as agonists, partial agonists or antagonists at the 5-HT_{1A} receptors, or act as serotonin reuptake inhibitors.

In one aspect, compounds of Formula I are described herein:

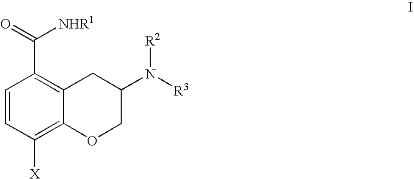

where X, R¹, R², and R³ are as described herein.

In another aspect, compounds of Formula II are described herein:

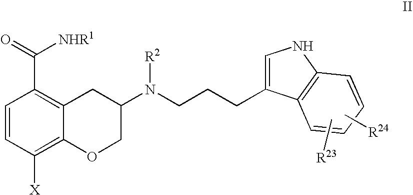

where X, R¹, R²³, and R²⁴ are as described herein.

In yet another aspect, compounds Formula III are described herein:

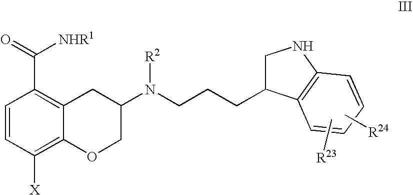

where X, R¹, R²³, and R²⁴ are as described herein.

In a further aspect, methods of making the compounds of Formulae I, II, or III are described herein.

In one aspect, compositions including a compound of Formulae I, II, or III and one or more pharmaceutically acceptable carriers are described herein.

In another aspect, methods of treating and/or preventing a serotonin-related disorder in a patient suspected of suffering from a serotonin-related disorder are described herein. The methods include the step of administering to the patient a therapeutically effective amount of a compound of Formulae I, II or III.

In yet another aspect, methods of agonizing 5-HT_{1A} receptors in a patient in need thereof are also described herein. The methods include the step of administering to the patient a therapeutically effective amount of a compound of Formulae I, II, or III.

In yet another aspect, methods of antagonizing 5-HT_{1A} receptors in a patient in need thereof are described herein. The methods include the step of administering to the patient a therapeutically effective amount of a compound of Formulae I, II, or III.

In a further aspect, methods of inhibiting the reuptake of serotonin in a patient in need thereof are described herein. The methods include the step of administering to the patient a therapeutically effective amount of a compound of Formulae I, II, or III.

### DETAILED DESCRIPTION

The term “(C₁-C₆)-alkyl” as used herein refers to a linear or branched, saturated hydrocarbon having from 1 to 6 carbon atoms. Representative (C₁-C₆)-alkyl groups include, but are not limited to methyl, ethyl, propyl, isopropyl, butyl, sec-butyl, tert-butyl, pentyl, isopentyl, neopentyl, hexyl, isohexyl, and neohexyl. In some embodiments, the (C₁-C₆)-alkyl group is optionally substituted with one or more of the following groups: halogen, —N₃, —NO₂, —CN, —OR′, —SR′, —SO₂R′, —SO₂N(R′)₂, —N(R′)₂, —COR′, —CO₂R′, —NR′CO₂R′, —NR′COR′, —NR′CONR′, or CON(R′)₂, where each R′ is independently hydrogen or unsubstituted (C₁-C₆)-alkyl. Similarly, “(C₁-C₃)-alkyl” as used herein refers to a linear or branched, saturated hydrocarbon, optionally substituted as described above, having from 1 to 3 carbon atoms. The carbon number, as used herein, refers to the carbon backbone and carbon branching, but does not include carbon atoms of the substituents, such as alkoxy substitutions and the like.

The term “haloalkyl” as used herein refers to an alkyl group where one or more of the alkyl group's hydrogen atoms has been replaced with a halogen atom, which may be the same or different. In some embodiments, n is 1 to 6, or 1 to 4 or 1 to 3. In some embodiments, n is 6, 5, 4, 3, 2, or 1. The one or more halogen atoms present in a haloalkyl group are located at any position in the group. For example, in some embodiments, the halogen atom(s) are located at the terminal portion of the group, while in other embodiments, the halogen atom(s) are located internally within the group or on the carbon atom bonded to the remainder of the molecule. Representative haloalkyl groups include, without limitation, —CH₃, —CF₃, —C₂F₅, —C₃H₄F₃, —C₄H₆F₃, —CHF₂, —CCl₃, —CHCl₂, —CH₂Cl, or —C₂Cl₅, —CH₂CH₂Br, —CH₂CH₂I, —CH₆F, —CH₆Cl, —CH₈Br, —CH₈I, —CH₁₀Br, —CH₁I, —CH₂CH(Br)CH₃, —CH₂CH(Cl)CH₂CH₃, —CH(F)CH₂CH₃, —C(CH₃)₂(CH₂Cl), —C₆H₁₂Br, and —C₆H₁₂I. “Haloalkyl” also includes alkyl groups where all of the hydrogen atoms are replaced with halogen atoms (i.e., perhaloalkyl groups).

The term “cycloalkyl”, as used herein, whether used alone or as part of another group refers to a substituted or unsubstituted saturated or unsaturated carbon ring having 3 to 6 carbon atoms; in one embodiment, 3 to 5 carbon atoms; in one embodiment, 3 to 4 carbon atoms. Any suitable ring position of the cycloalkyl group may be covalently linked to the defined chemical structure. Exemplary cycloalkyl groups include, but are not limited to cyclopropyl, cyclobutyl, cyclopentyl, cyclohexyl, —CH₂—(C₃H₅), and —CH₂—(C₄H₇). In one embodiment, the cycloalkyl group is substituted with one or more of the following groups: oxo (i.e., ═O), —V-halogen, —V—N₃, —V—NO₂, —V—CN, —V—OR′, —V—SR′, —V—SO₂R′, —V—SO₂N(R′)₂, —V—N(R′)₂, —V—COR′, —V—CO₂R′, —V—NR′CO₂R′, —V—NR′COR′, —V—NR′CONR′, or —V—CON(R′)₂, where each R′ is independently hydrogen or unsubstituted (C₁-C₆)-alkyl; and where each V is independently a bond or (C₁-C₆)-alkyl.

The terms “heterocycle” refer to fully saturated, or partially or fully unsaturated, including aromatic (i.e., “heteroaryl”) cyclic groups (for example, 4 to 7 membered monocyclic, 7 to 11 membered bicyclic, or 8 to 16 membered tricyclic ring systems) which have at least one heteroatom in at least one carbon atom-containing ring. Each ring of the heterocyclic group containing a heteroatom may have 1, 2, 3, or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. (The term “heteroarylium” refers to a heteroaryl group bearing a quaternary nitrogen atom and thus a positive charge.) The heterocyclic group may be attached to the remainder of the molecule at any heteroatom or carbon atom of the ring or ring system. Exemplary monocyclic heterocyclic groups include azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include indolyl, isoindolyl, benzothiazolyl, benzoxazolyl, benzoxadiazolyl, benzothienyl, benzo[d][1,3]dioxolyl, 2,3-dihydrobenzo[b][1,4]dioxinyl, quinuclidinyl, quinolinyl, tetrahydroisoquinolinyl, isoquinolinyl, benzimidazolyl, benzopyranyl, indolizinyl, benzofuryl, benzofurazanyl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl and the like. Exemplary tricyclic heterocyclic groups include carbazolyl, benzidolyl, phenanthrolinyl, acridinyl, phenanthridinyl, xanthenyl and the like.

“Substituted heterocycle” and “substituted heterocyclic” (such as “substituted heteroaryl”) refer to heterocycle or heterocyclic groups substituted with one or more substituents, preferably 1 to 4 substituents, at any available point of attachment. Exemplary substituents include, but are not limited to, cycloalkyl or substituted cycloalkyl, oxo (i.e., ═O), alkyl or substituted alkyl, as well as those groups recited above as exemplary cycloalkyl substituents.

The term “aryl” or “ara” (as in “aralkyl”) refers to cyclic, aromatic hydrocarbon groups that have 1 to 5 aromatic rings, especially monocyclic or bicyclic groups such as phenyl, biphenyl or naphthyl. Where containing two or more aromatic rings (bicyclic, etc.), the aromatic rings of the aryl group may be joined at a single point (e.g., biphenyl), or fused (e.g., naphthyl, phenanthrenyl and the like). “Substituted aryl” refers to an aryl group substituted by one or more substituents, preferably 1 to 3 substituents, at any point of attachment. Exemplary substituents include, but are not limited to, cycloalkyl or substituted cycloalkyl, alkyl or substituted alkyl, as well as those groups recited above as exemplary cycloalkyl substituents. The exemplary substitutents can themselves be optionally substituted. Exemplary substituents also include fused cylic groups, especially fused cycloalkyl, fused cycloalkenyl, fused heterocycle, or fused aryl, where the aforementioned cycloalkyl, cycloalkenyl, heterocycle and aryl substituents can themselves be optionally substituted.

The term “alkoxy”, as used herein, whether used alone or as part of another group, refers to the group —O—Rₐ, where Rₐ is an alkyl group containing 1 to 6 carbon atoms; in one embodiment 1 to 5 carbon atoms; in one embodiment 1 to 4 carbon atoms; in one embodiment, 1 to 3 carbon atoms.

The term “halogen” as used herein refers to fluorine, chlorine, bromine, and iodine.

The term “administer”, “administering”, or “administration”, as used herein refers to either directly administering a compound or pharmaceutically acceptable salt of the compound or a composition to an animal, or administering a prodrug derivative or analog of the compound or pharmaceutically acceptable salt of the compound or composition to the animal, which can form an equivalent amount of the compound within the animal's body.

The term “animal” as used herein includes, without limitation, a human, mouse, rat, guinea pig, dog, cat, horse, cow, pig, monkey, chimpanzee, baboon, or rhesus. In one embodiment, the animal is a mammal. In another embodiment, the animal is a human.

The term “conditions effective to” as used herein refers to synthetic reaction conditions that will be apparent to those skilled in the art of synthetic organic chemistry.

The term “effective amount” as used herein refers to an amount of a compound or pharmaceutically acceptable salt of a compound that, when administered to an animal, is effective to prevent, to at least partially ameliorate, or to cure, a condition from which the animal suffers or is suspected to suffer.

The term “isolated” as used herein refers to separate from other components of a reaction mixture, or a natural source. In certain embodiments, the isolate contains at least about 50%, at least about 55%, at least about 60%, at least about 65%, at least about 70%, at least about 75%, at least about 80%, at least about 85%, at least about 90%, at least about 95%, or at least about 98% of the compound or pharmaceutically acceptable salt of the compound by weight of the isolate.

The term “pharmaceutically acceptable salt”, as used herein, refers to salts derived from organic and inorganic acids of a compound described herein. Exemplary salts include, but are not limited to, sulfate, citrate, acetate, oxalate, chloride, hydrochloride, bromide, hydrobromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, succinate, fumarate, maleate, malonate, mandelate, malate, phthalate, and pamoate. The term “pharmaceutically acceptable salt” as used herein also refers to a salt of a compound described herein having an acidic functional group, such as a carboxylic acid functional group, and a base. Exemplary bases include, but are not limited to, hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like. The term “pharmaceutically acceptable salt” also includes hydrates of a compound described herein.

The term “substantially free of the corresponding opposite enantiomer” as used herein means that the compound contains no more than about 10% by weight of its corresponding opposite enantiomer. In other embodiments, the compound that is substantially free of its corresponding opposite enantiomer contains no more than about 50%, no more than about 25%, no more than about 10%, no more than about 5%, no more than about 1%, no more than about 0.5%, or no more than about 0.1% by weight of its corresponding opposite enantiomer. An enantiomer that is substantially free of its corresponding opposite enantiomer includes a compound that has been isolated and purified or has been prepared substantially free of its corresponding opposite enantiomer.

The term “carrier”, as used herein, shall encompass carriers, excipients, and diluents.

When ranges are used herein for physical properties, such as molecular weight, or chemical properties, such as chemical Formulae, all combinations and subcombinations of ranges of specific embodiments therein are intended to be included.

The disclosure of each patent, patent application, and publication cited or described in this document is hereby incorporated by reference, in their entirety.

In certain embodiments, compounds of Formula I are provided:

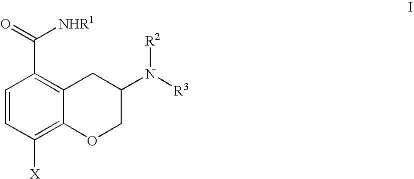

or an enantiomer, diastereomer, tautomer, or pharmaceutically acceptable salt or solvate thereof, wherein:

X is (C₁-C₆)-alkyl, —OR¹², —SR¹², or —NR¹³R¹⁴

R¹ is hydrogen, (C₁-C₃)-alkyl, cycloalkyl, cycloalkylalkyl, phenyl, or benzyl;

R² is hydrogen, (C₁-C₄)-alkyl, (C₃-C₆)-cycloalkyl, cycloalkylalkyl, aralkyl, or haloalkyl;

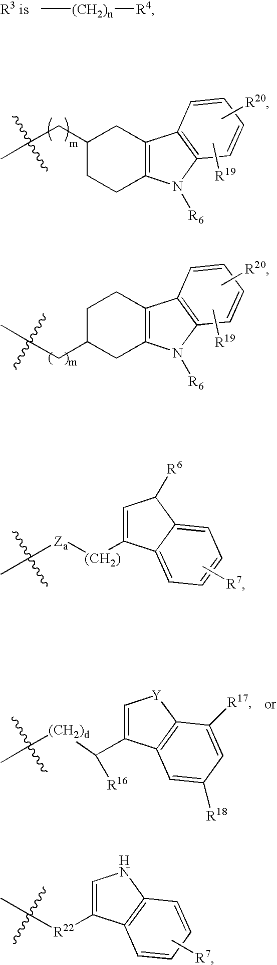

n is an integer of 2 to 4; and R⁴ is A, B, C, D, E, G, J, K, L, P, U, AA or BB, provided that when n is an integer of 2, then R⁴ is A, B, C, D, E, G, J, K, L, U, AA or BB; and further provided that when n is an integer of 3 or 4, then R⁴ is A, B, C, D, K, L, P, U, AA or BB;

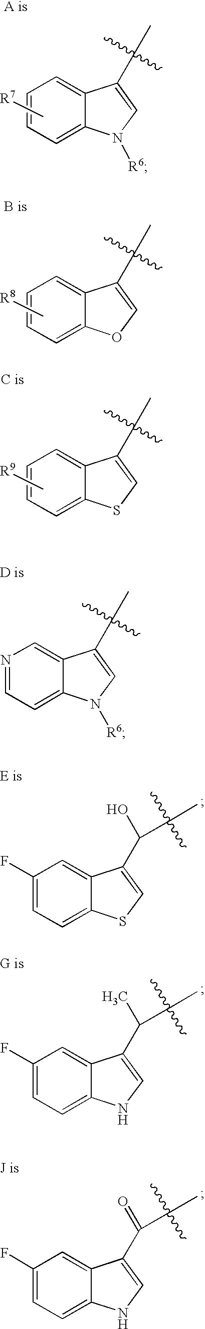

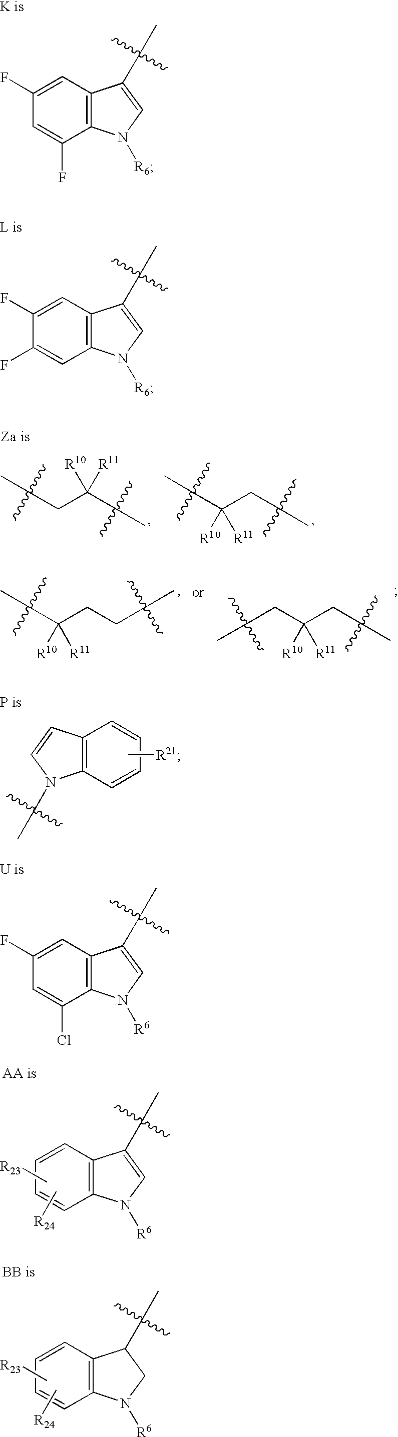

R⁶ is hydrogen or (C₁-C₆)-alkyl;

R⁷ is hydrogen, fluoro, chloro, cyano, or alkoxy;

R⁸ is hydrogen, halo, (C₁-C₃)-alkoxy or (C₁-C₃)-alkyl;

R⁹ is hydrogen, halo, (C₁-C₃)-alkoxy or (C₁-C₃)-alkyl;

R¹⁰ is hydrogen or methyl;

R¹¹ is methyl;

R¹², R¹³ and R¹⁴ are each independently hydrogen or (C₁-C₆)-alkyl, R¹³ and R¹⁴ together with the N to which they are bonded optionally form a heterocycle;

R¹⁹ and R²⁰ are independently hydrogen, fluoro, chloro, cyano, or (C₁-C₆)-alkyl;

R²¹ is hydrogen or fluoro;

R²² is a 3- to 7-membered saturated carbocyclic ring;

R²³ and R²⁴ are independently hydrogen, halogen, cyano, or (C₁-C₆)-alkyl;

m is an integer of 1 or 2;

Y is O, S, or NH;

provided that when Y is O, then R¹⁶ is hydrogen; R¹⁷ is hydrogen or OCH₃; R¹⁸ is hydrogen; and d is an integer of 2 or 3;

further provided that when Y is S, then R¹⁶ is hydrogen or hydroxyl; R¹⁷ is hydrogen; R¹⁸ is hydrogen or fluoro; and d is an integer of 2;

further provided that when Y is NH, then R¹⁶ is keto (═O) or methyl; R¹⁷ is hydrogen; R¹⁸ is fluoro; and d is an integer of 2.

In some embodiments, X is (C₁-C₃)-alkyl, —OR¹², —SR¹², or —NR¹³R¹⁴. In some embodiments, X is methyl, —OR¹², —SR¹², or —NR¹³R¹⁴. In some embodiments, X is (C₁-C₃)-alkyl or —OR¹². In some embodiments, X is (C₁-C₃)-alkyl or —OCH₃.

In some embodiments, R¹², R¹³, and R¹⁴ are each independently hydrogen or (C₁-C₃ alkyl), or R¹³ and R¹⁴ together with the N to which they are bonded optionally form a substituted or unsubstituted

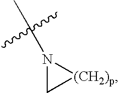

in which p is an integer selected from the group consisting of 1, 2, 3, 4 and 5. In some embodiments, R¹² is hydrogen or (C₁-C₃)-alkyl and R¹³ and R¹⁴ are each independently hydrogen or methyl.

In some embodiments, X is (C₁-C₃)-alkyl, —OR¹², —SR¹², or —NR¹³R¹⁴; and R¹²R¹³, and R¹⁴ are each independently hydrogen or (C₁-C₃)-alkyl. In some embodiments, X is methyl, —OR¹², —SR¹², or —NR¹³R¹⁴; and R¹², R¹³, and R¹⁴ are each independently hydrogen or (C₁-C₃)-alkyl. In some embodiments, X is methyl, —OR¹², —SR¹², or —NR¹³ R¹⁴; and R¹² is hydrogen or (C₁-C₃)-alkyl; and R¹³, and R¹⁴ are each independently hydrogen or methyl. In some embodiments, X is —OR¹² and R¹² is hydrogen or (C₁-C₃)-alkyl. In some embodiments, X is —SR¹² and R¹² is hydrogen or methyl. In some embodiments, X is —NR¹³R¹⁴ and R¹³ and R¹⁴ are each independently hydrogen or methyl. In some embodiments, X is (C₁-C₃)-alkyl, —OH, —OCH₃, —OCH₂CH₃, —OCH₂CH₂CH₃, —OCH(CH₃)₂, —SCH₃, —SCH₂CH₃, —NHCH₃, —NHCH₂CH₃, —N(CH₃)₂, —N(CH₃)(CH₂CH₃), or —N(CH₂CH₃)₂.

In some embodiments, R¹ is hydrogen or (C₁-C₃)-alkyl. In some embodiments, R¹ is hydrogen or methyl, ethyl, propyl or isopropyl. In some embodiments, R¹ is hydrogen or methyl.

In some embodiments, R² is hydrogen, (C₁-C₆)-alkyl, (C₃-C₆)-cycloalkyl, cycloalkylalkyl, alkylcycloalkyl, or haloalkyl. In some embodiments, R² is hydrogen, (C₁-C₆)-alkyl, (C₃-C₆)-cycloalkyl, cycloalkylalkyl, or alkylcycloalkyl. In some embodiments, R² is hydrogen, (C₁-C₄)-alkyl, (C₃-C₄)-cycloalkyl, cycloalkylalkyl, alkylcycloalkyl, or trifluoro-(C₁-C₄)-alkyl. In some embodiments, R² is hydrogen, (C₁-C₄)-alkyl, (C₃-C₄)-cycloalkyl, (C₃-C₆)-cycloalkyl-(C₁-C₆)alkyl, (C₁-C₆)-alkyl-(C₃-C₆)-cycloalkyl or halo-(C₁-C₆)-alkyl. In some embodiments, R² is hydrogen, (C₁-C₄)-alkyl, (C₃-C₄)-cycloalkyl, (C₃-C₄)-cycloalkyl-(C₁-C₃)alkyl, (C₁-C₃)alkyl-(C₃-C₄)-cycloalkyl, or halo-(C₁-C₄)-alkyl. In some embodiments, R² is hydrogen, (C₁-C₄)-alkyl, (C₃-C₄)-cycloalkyl, (C₃-C₄)-cycloalkyl-(C₁)-alkyl, (C₁)-alkyl-(C₃-C₄)-cycloalkyl, or halo-(C₁-C₄)-alkyl where the alkyl chain is partially or fully substituted with halogen atoms. In some embodiments, R² is hydrogen, methyl, ethyl, propyl, isopropyl, butyl, isobutyl, cyclopropyl, cyclobutyl, cyclopropylmethyl, cyclopropylethyl, cyclopropylpropyl, cyclobutylmethyl, cyclobutylethyl, cyclobutylpropyl, methylcyclopropyl, —CF₃, —CH₂CF₃, —CH₂CH₂CF₃, or —CH₂CH₂CH₂CF₃. In some embodiments, R² is hydrogen, methyl, ethyl, propyl, isopropyl, butyl, isobutyl, cyclobutyl, cyclopropylmethyl, methylcyclopropyl, —CH₂CH₂CF₃, or —CH₂CH₂CH₂CF₃. In some embodiments, R² is hydrogen, ethyl, propyl, cyclobutyl, cyclopropylmethyl, or methylcyclopropyl.

In some embodiments, R³ is —(CH₂)ₙ—R⁴; n is an integer of 2 to 4; and R⁴ is

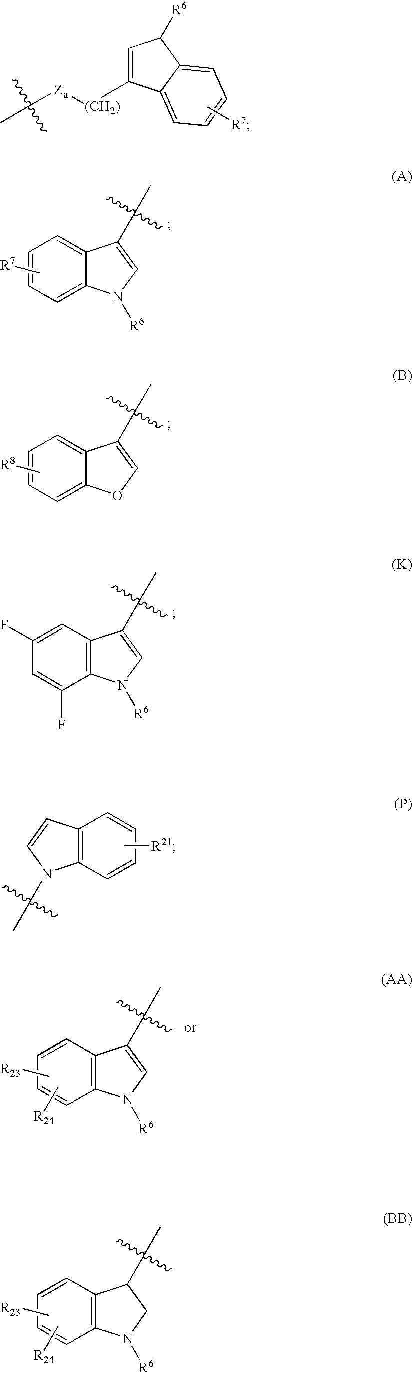

In some embodiments, R⁴ is, K, A, AA or BB. In some embodiments, R⁴ is AA or BB.

In some embodiments, R⁶ is hydrogen or alkyl. In some embodiments, R⁶ is hydrogen or (C₁-C₆)-alkyl. In some embodiments, R⁶ is hydrogen or (C₁-C₃)-alkyl. In some embodiments, R⁶ is hydrogen or methyl.

In some embodiments, R⁷ is hydrogen, fluoro, or cyano at any of the 5-, 6-, or 7-position. In some embodiments, R⁷ is hydrogen, fluoro, or cyano at the 5-position.

In some embodiments, R⁸ is hydrogen or —OR¹², where R¹² is (C₁-C₃)-alkyl. In some embodiments, R⁸ is hydrogen or —OCH₃.

In some embodiments, R⁹ is hydrogen or fluoro.

In some embodiments, R²³ and R²⁴ are each independently hydrogen, fluoro, chloro, bromo, or iodo.

In some embodiments, R²³ and R²⁴ are each independently hydrogen or fluoro.

In some embodiments, Zₐ is

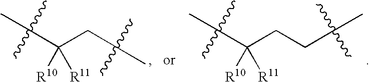

In some embodiments, R¹⁶ is hydrogen when Y is O or S, or methyl when Y is NH.

In some embodiments, R¹⁷ is hydrogen when Y is O or S, or NH. In some embodiments, R¹⁷ is methoxy when Y is O.

In some embodiments, R¹⁹ and R²⁰ are each fluoro.

In some embodiments, R²¹ is fluoro.

In some embodiments, R²² is a 4-, 5-, or 6-membered ring.

In some embodiments, n is 3.

In some embodiments, n is 3 and R⁷ is fluoro. In some embodiments, n is 3 and R⁷ is fluoro at the 5-position. In some embodiments, n is 3 and one of R₂₃ and R₂₄ is fluoro and the other of R₂₃ and R₂₄ is hydrogen. In some embodiments, n is 3 and both R₂₃ and R₂₄ are fluoro. In some embodiments, n is 3 and R₂₃ and R₂₄ are fluoro at the 5 and 7 positions.

In certain embodiments, compounds or pharmaceutically acceptable salts of Formula II are provided

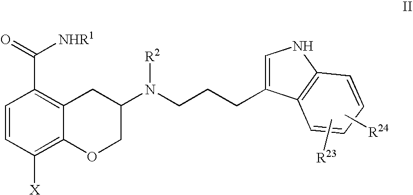

where X, R¹, R², R¹², R¹³, R¹⁴ R²³ and R²⁴ are as defined herein.

In certain other embodiments, compounds or pharmaceutically acceptable salts of Formula III are provided:

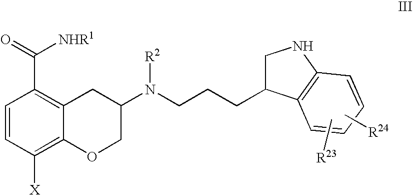

where X, R¹, R², R¹², R¹³, R¹⁴ R²³ and R²⁴ are as defined herein.

In some embodiments, X of Formula II or III is methyl, —OR¹², —SR¹², or —NR¹³R¹⁴.

In some embodiments, R¹², R¹³, and R¹⁴ of Formula II or III are each independently hydrogen or (C₁-C₃)-alkyl. In some cases, R¹³ and R¹⁴ together with the N to which they are bonded optionally form a substituted or unsubstituted

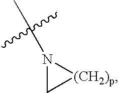

in which p is an integer selected from the group consisting of 1, 2, 3, 4 and 5. In some embodiments, R¹² is hydrogen or (C₁-C₃)-alkyl and R¹³ and R¹⁴ are each independently hydrogen or methyl.

In some embodiments, X of Formula II or III is (C₁-C₃)-alkyl, —OR¹², —SR¹², or —NR¹³R¹⁴; and R¹², R¹³, and R¹⁴ are each independently hydrogen or (C₁-C₃)-alkyl. In some embodiments, X is methyl, —OR¹², —SR¹², or —NR¹³R¹⁴; and R¹², R¹³, and R¹⁴ are each independently hydrogen or (C₁-C₃)-alkyl. In some embodiments, X is methyl, —OR¹², —SR¹², or —NR¹³R¹⁴; and R¹² is hydrogen or (C₁-C₃)-alkyl; and R¹³, and R¹⁴ are each independently hydrogen or methyl. In some embodiments, X is —OR¹² and R¹² is hydrogen or (C₁-C₃)-alkyl. In some embodiments, X is —SR¹² and R¹² is hydrogen or methyl. In some embodiments, X is —NR¹³R¹⁴ and R¹³ and R¹⁴ are each independently hydrogen or methyl. In some cases, R¹³ and R¹⁴ together with the N to which they are bonded optionally form a substituted or unsubstituted

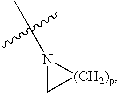

in which p is an integer selected from the group consisting of 1, 2, 3, 4 and 5. In some embodiments, X is (C₁-C₃)-alkyl, —OH, —OCH₃, —OCH₂CH₃, —OCH₂CH₂CH₃, —OCH(CH₃)₂, —SCH₃, —SCH₂CH₃, —NHCH₃, —NHCH₂CH₃, —N(CH₃)₂, —N(CH₃)(CH₂CH₃), or —N(CH₂CH₃)₂.

In some embodiments, R¹ of Formula II or III is hydrogen or (C₁-C₃)-alkyl. In some embodiments, R¹ is hydrogen or methyl, ethyl, propyl or isopropyl. In some embodiments, R¹ is hydrogen or methyl.

In some embodiments, R² of Formula II or III is hydrogen, (C₁-C₆)-alkyl, (C₃-C₆)-cycloalkyl, cycloalkylalkyl, or haloalkyl. In some embodiments, R² is hydrogen, (C₁-C₄)-alkyl, (C₃-C₄)-cycloalkyl, cycloalkylalkyl, or trifluoro-(C₁-C₄)-alkyl. In some embodiments, R² is hydrogen, (C₁-C₄)-alkyl, (C₃-C₄)-cycloalkyl, (C₃-C₆)-cycloalkyl-(C₁-C₆)alkyl, or halo-(C₁-C₆)-alkyl. In some embodiments, R² is hydrogen, (C₁-C₄)-alkyl, (C₃-C₄)-cycloalkyl, (C₃-C₄)-cycloalkyl-(C₁-C₃)alkyl, or halo-(C₁-C₄)-alkyl. In some embodiments, R² is hydrogen, (C₁-C₄)-alkyl, (C₃-C₄)-cycloalkyl, (C₃-C₄)-cycloalkyl-(C₁)-alkyl, or halo-(C₁-C₄)-alkyl where the alkyl chain is partially or fully substituted with halogen atoms. In some embodiments, R² is hydrogen, methyl, ethyl, propyl, isopropyl, butyl, isobutyl, cyclopropyl, cyclobutyl, cyclopropylmethyl, cyclopropylethyl, cyclopropylpropyl, cyclobutylmethyl, cyclobutylethyl, cyclobutylpropyl, —CF₃, —CH₂CF₃, —CH₂CH₂CF₃, or —CH₂CH₂CH₂CF₃. In some embodiments, R² is hydrogen, methyl, ethyl, propyl, isopropyl, butyl, isobutyl, cyclobutyl, cyclopropylmethyl, —CH₂CH₂CF₃, or —CH₂CH₂CH₂CF₃.

In some embodiments of Formulae II or III, R²³ and R²⁴ are each independently hydrogen, fluoro, chloro, bromo, or iodo. In some embodiments, R²³ and R²⁴ are each independently hydrogen or fluoro.

Specific examples of compounds of Formulae I, II or III include, without limitation:
3-{[3-(5-fluoro-1H-indol-3-yl)propyl]amino}-8-methoxychromane-5-carboxamide;
(+)-3-{[3-(5-fluoro-1H-indol-3-yl)propyl]amino}-8-methoxychromane-5-carboxamide;
(−)-3-{[3-(5-fluoro-1H-indol-3-yl)propyl]amino}-8-methoxychromane-5-carboxamide;
3-{cyclobutyl[3-(5-fluoro-1H-indol-3-yl)propyl]amino}-8-methoxychromane-5-carboxamide;
(+)-3-{cyclobutyl[3-(5-fluoro-1H-indol-3-yl)propyl]amino}-8-methoxychromane-5-carboxamide;
(−)₃-{cyclobutyl[3-(5-fluoro-1H-indol-3-yl)propyl]amino}-8-methoxychromane-5-carboxamide;
3-{cyclopropylmethyl[3-(5-fluoro-1H-indol-3-yl)propyl]amino}-8-methoxychromane-5-carboxamide;
(−)-3-{cyclopropylmethyl[3-(5-fluoro-1H-indol-3-yl)propyl]amino}-8-methoxy chromane-5-carboxamide;
(+)-3-{cyclopropylmethyl[3-(5-fluoro-1H-indol-3-yl)propyl]amino}-8-methoxy chromane-5-carboxamide;
3-{cyclobutyl[3-(5-fluoro-1H-indol-3-yl)propyl]amino}-8-methylchromane-5-carboxamide;
(+)-3-{cyclobutyl[3-(5-fluoro-1H-indol-3-yl)propyl]amino}-8-methylchromane-5-carboxamide;
(−)-3-{cyclobutyl[3-(5-fluoro-1H-indol-3-yl)propyl]amino}-8-methylchromane-5-carboxamide;
3-{cyclobutyl[3-(5-fluoro-1H-indol-3-yl)propyl]amino}-8-ethylchromane-5-carboxamide;
(+)-3-{cyclobutyl[3-(5-fluoro-1H-indol-3-yl)propyl]amino}-8-ethylchromane-5-carboxamide;
(−)-3-{cyclobutyl[3-(5-fluoro-1H-indol-3-yl)propyl]amino}-8-ethylchromane-5-carboxamide;
3-{cyclobutyl[3-(5,7-difluoro-1H-indol-3-yl)propyl]amino}-8-methoxychromane-5-carboxamide;
(−)-3-{cyclobutyl[3-(5,7-difluoro-1H-indol-3-yl)propyl]amino}-8-methoxychromane-5-carboxamide;
(+)-3-{cyclobutyl[3-(5,7-difluoro-1H-indol-3-yl)propyl]amino}-8-methoxychromane-5-carboxamide;
3-{[(5-fluoro-1H-indol-3-yl)-1-propyl](propyl)amino}-8-methoxychromane-5-carboxamide;
(+)-3-{[(5-fluoro-1H-indol-3-yl)-1-propyl](propyl)amino}-8-methoxychromane-5-carboxamide;
(−)-3-{[(5-fluoro-1H-indol-3-yl)-1-propyl](propyl)amino}-8-methoxychromane-5-carboxamide;
3-{cyclobutyl[3-(5-fluoro-1H-indol-3-yl)propyl]amino}-8-methylaminochromane-5-carboxamide;
3-{cyclobutyl[3-(5-fluoro-1H-indol-3-yl)propyl]amino}-8-(methylthio)chromane-5-carboxamide;
3-{cyclobutyl[3-(5-fluoro-1H-indol-3-yl)propyl]amino}-8-dimethylaminochromane-5-carboxamide;
3-{ethyl[3-(5-fluoro-1H-indol-3-yl)propyl]amino}-8-methoxychromane-5-carboxamide;
3-{[3-(5-fluoro-1H-indolin-3-yl)propyl]amino}-8-methoxychromane-5-carboxamide;
(+)-3-{[3-(5-fluoro-1H-indolin-3-yl)propyl]amino}-8-methoxychromane-5-carboxamide;
(−)-3-{[3-(5-fluoro-1H-indolin-3-yl)propyl]amino}-8-methoxychromane-5-carboxamide;
3-{cyclobutyl[3-(5-fluoro-1H-indolin-3-yl)propyl]amino}-8-methoxychromane-5-carboxamide;
(+)-3-{cyclobutyl[3-(5-fluoro-1H-indolin-3-yl)propyl]amino)-8-methoxychromane-5-carboxamide;
(−)₃-{cyclobutyl[3-(5-fluoro-1H-indolin-3-yl)propyl]amino)-8-methoxychromane-5-carboxamide;
3-{cyclopropylmethyl[3-(5-fluoro-1H-indolin-3-yl)propyl]amino}-8-methoxychromane-5-carboxamide;
(−)-3-{cyclopropylmethyl[3-(5-fluoro-1H-indolin-3-yl)propyl]amino}-8-methoxy chromane-5-carboxamide;
(+)-3-{cyclopropylmethyl[3-(5-fluoro-1H-indolin-3-yl)propyl]amino}-8-methoxy chromane-5-carboxamide;
3-{cyclobutyl[3-(5-fluoro-1H-indolin-3-yl)propyl]amino}-8-methylchromane-5-carboxamide;
(+)-3-{cyclobutyl[3-(5-fluoro-1H-indolin-3-yl)propyl]amino}-8-methylchromane-5-carboxamide;
(−)-3-{cyclobutyl[3-(5-fluoro-1H-indolin-3-yl)propyl]amino}-8-methylchromane-5-carboxamide;
3-{cyclobutyl[3-(5-fluoro-1H-indolin-3-yl)propyl]amino}-8-ethylchromane-5-carboxamide;
(+)-3-{cyclobutyl[3-(5-fluoro-1H-indolin-3-yl)propyl]amino}-8-ethylchromane-5-carboxamide;
(−)-3-{cyclobutyl[3-(5-fluoro-1H-indolin-3-yl)propyl]amino}-8-ethylchromane-5-carboxamide;
3-{cyclobutyl[3-(5,7-difluoro-1H-indolin-3-yl)propyl]amino}-8-methoxychromane-5-carboxamide;
(−)-3-{cyclobutyl[3-(5,7-difluoro-1H-indolin-3-yl)propyl]amino}-8-methoxychromane-5-carboxamide;
(+)-3-{cyclobutyl[3-(5,7-difluoro-1H-indolin-3-yl)propyl]amino}-8-methoxychromane-5-carboxamide;
3-{[4-(5-fluoro-1H-indolin-3-yl)-1-propyl](propyl)amino}-8-methoxychromane-5-carboxamide;
(+)-3-{[4-(5-fluoro-1H-indolin-3-yl)-1-propyl](propyl)amino}-8-methoxy-chromane-5-carboxamide;
(−)-3-{[4-(5-fluoro-1H-indolin-3-yl)-1-propyl](propyl)amino}-8-methoxychromane-5-carboxamide;
3-{cyclobutyl[3-(5-fluoro-1H-indolin-3-yl)propyl]amino}-8-methylaminochromane-5-carboxamide;
(+)-3-{cyclobutyl[3-(5-fluoro-1H-indolin-3-yl)propyl]amino}-8-methylaminochromane-5-carboxamide;
(−)-3-{cyclobutyl[3-(5-fluoro-1H-indolin-3-yl)propyl]amino}-8-methylaminochromane-5-carboxamide;
3-{cyclobutyl[3-(5-fluoro-1H-indolin-3-yl)propyl]amino}-(8-methylthio)-chromane-5-carboxamide;
3-{cyclobutyl[3-(5-fluoro-1H-indolin-3-yl)propyl]amino}-(8-methylthio)-chromane-5-carboxamide;
(+)-3-{cyclobutyl[3-(5-fluoro-1H-indolin-3-yl)propyl]amino}-(8-methylthio)-chromane-5-carboxamide;
(−)-3-{cyclobutyl[3-(5-fluoro-1H-indolin-3-yl)propyl]amino}-(8-methylthio)-chromane-5-carboxamide;
3-{cyclobutyl[3-(5-fluoro-1H-indolin-3-yl)propyl]amino}-8-dimethylaminochromane-5-carboxamide;
(+)-3-{cyclobutyl[3-(5-fluoro-1H-indolin-3-yl)propyl]amino}-8-dimethylaminochromane-5-carboxamide;
(−)-3-{cyclobutyl[3-(5-fluoro-1H-indolin-3-yl)propyl]amino}-8-dimethylaminochromane-5-carboxamide;
3-{ethyl[3-(5-fluoro-1H-indolin-3-yl)propyl]amino}-8-methoxychromane-5-carboxamide
(+)-3-{ethyl[3-(5-fluoro-1H-indolin-3-yl)propyl]amino}-8-methoxychromane-5-carboxamide;
(−)-3-{ethyl[3-(5-fluoro-1H-indolin-3-yl)propyl]amino}-8-methoxychromane-5-carboxamide;
3-{cyclobutyl[3-(5-fluoro-1H-indol-3-yl)propyl]amino}-8-ethoxychromane-5-carboxamide;
(3R)-3-{cyclobutyl[3-(5-fluoro-1H-indol-3-yl)propyl]amino}-8-ethoxychromane-5-carboxamide;
(+)-3-{cyclobutyl[3-(5-fluoro-1H-indol-3-yl)propyl]amino}-8-ethoxychromane-5-carboxamide;
(−)-3-{cyclobutyl[3-(5-fluoro-1H-indol-3-yl)propyl]amino}-8-ethoxychromane-5-carboxamide;
3-{cyclobutyl[3-(5-fluoro-1H-indol-3-yl)propyl]amino}-8-propoxychromane-5-carboxamide;
(3R)-3-{cyclobutyl[3-(5-fluoro-1H-indol-3-yl)propyl]amino}-8-propoxychromane-5-carboxamide;
(+)-3-{cyclobutyl[3-(5-fluoro-1H-indol-3-yl)propyl]amino}-8-propoxychromane-5-carboxamide;
(−)-3-{cyclobutyl[3-(5-fluoro-1H-indol-3-yl)propyl]amino}-8-propoxychromane-5-carboxamide;
3-{cyclobutyl[3-(5-fluoro-1H-indol-3-yl)propyl]amino}-8-isopropoxychromane-5-carboxamide;
(+)-3-{cyclobutyl[3-(5-fluoro-1H-indol-3-yl)propyl]amino}-8-isopropoxychromane-5-carboxamide;
(−)-3-{cyclobutyl[3-(5-fluoro-1H-indol-3-yl)propyl]amino}-8-isopropoxychromane-5-carboxamide;
3-{cyclobutyl[3-(5-fluoro-1H-indol-3-yl)propyl]amino}-8-isopropoxychromane-5-carboxamide;
(3R)-3-{cyclobutyl[3-(5-fluoro-1H-indol-3-yl)propyl]amino}-8-isopropoxychromane-5-carboxamide;
(+)-3-{cyclobutyl[3-(5-fluoro-1H-indol-3-yl)propyl]amino}-8-isopropoxychromane-5-carboxamide;
(−)-3-{cyclobutyl[3-(5-fluoro-1H-indol-3-yl)propyl]amino}-8-isopropoxychromane-5-carboxamide;
3-{cyclobutyl[3-(5-fluoro-1H-indol-3-yl)propyl]amino}-8-hydroxychromane-5-carboxamide;
(3R)-3-{cyclobutyl[3-(5-fluoro-1H-indol-3-yl)propyl]amino}-8-hydroxychromane-5-carboxamide;
(+)-3-{cyclobutyl[3-(5-fluoro-1H-indol-3-yl)propyl]amino}-8-hydroxychromane-5-carboxamide;
(−)-3-{cyclobutyl[3-(5-fluoro-1H-indol-3-yl)propyl]amino}-8-hydroxychromane-5-carboxamide;
3-{(3,3,3-trifluoropropyl)-[3-(5-fluoro-1H-indol-3-yl)propyl]amino}-8-methoxychromane-5-carboxamide;
(3R)-3-{(3,3,3-trifluoropropyl)-[3-(5-fluoro-1H-indol-3-yl)propyl]amino}-8-methoxychromane-5-carboxamide;
(+)-3-{(3,3,3-trifluoropropyl)-[3-(5-fluoro-1H-indol-3-yl)propyl]amino}-8-methoxychromane-5-carboxamide;
(−)-3-{(3,3,3-trifluoropropyl)-[3-(5-fluoro-1H-indol-3-yl)propyl]amino}-8-methoxychromane-5-carboxamide;
3-{isopropyl[3-(5-fluoro-1H-indol-3-yl)propyl]amino}-8-methoxychromane-5-carboxamide;
(3R)-3-{isopropyl[3-(5-fluoro-1H-indol-3-yl)propyl]amino}-8-methoxychromane-5-carboxamide;
(+)-3-{isopropyl[3-(5-fluoro-1H-indol-3-yl)propyl]amino}-8-methoxychromane-5-carboxamide;
(−)-3-{isopropyl[3-(5-fluoro-1H-indol-3-yl)propyl]amino}-8-methoxychromane-5-carboxamide;
-3-{butyl[3-(5-fluoro-1H-indol-3-yl)propyl]amino}-8-methoxychromane-5-carboxamide;
(3R)-3-{butyl[3-(5-fluoro-1H-indol-3-yl)propyl]amino}-8-methoxychromane-5-carboxamide;
(+)-3-{butyl[3-(5-fluoro-1H-indol-3-yl)propyl]amino}-8-methoxychromane-5-carboxamide;
(−)-3-{butyl[3-(5-fluoro-1H-indol-3-yl)propyl]amino}-8-methoxychromane-5-carboxamide;
(3R)-3-{4,4,4-trifluorobutyl[3-(5-fluoro-1H-indol-3-yl)propyl]amino}-8-methoxychromane-5-carboxamide;
3-{2-isobutyl[3-(5-fluoro-1H-indol-3-yl)propyl]amino}-8-methoxychromane-5-carboxamide;
(3R)-3-{2-isobutyl[3-(5-fluoro-1H-indol-3-yl)propyl]amino}-8-methoxychromane-5-carboxamide;
(+)-3-{2-isobutyl[3-(5-fluoro-1H-indol-3-yl)propyl]amino}-8-methoxychromane-5-carboxamide;
(−)-3-{2-isobutyl[3-(5-fluoro-1H-indol-3-yl)propyl]amino}-8-methoxychromane-5-carboxamide;
3-{methyl[3-(5-fluoro-1H-indol-3-yl)propyl]amino}-8-methoxychromane-5-carboxamide;
(3R)-3-{methyl[3-(5-fluoro-1H-indol-3-yl)propyl]amino}-8-methoxychromane-5-carboxamide;
(+)-3-{methyl[3-(5-fluoro-1H-indol-3-yl)propyl]amino}-8-methoxychromane-5-carboxamide;
(−)-3-{methyl[3-(5-fluoro-1H-indol-3-yl)propyl]amino}-8-methoxychromane-5-carboxamide;
3-{cyclobutyl[3-(5-fluoro-1H-indol-3-yl)propyl]amino}-8-methoxychromane-5-methylcarboxamide;
(3R)-3-{cyclobutyl[3-(5-fluoro-1H-indol-3-yl)propyl]amino}-8-methoxychromane-5-methylcarboxamide;
(+)-3-{cyclobutyl[3-(5-fluoro-1H-indol-3-yl)propyl]amino}-8-methoxychromane-5-methylcarboxamide;
(−)-3-{cyclobutyl[3-(5-fluoro-1H-indol-3-yl)propyl]amino}-8-methoxychromane-5-methylcarboxamide;
3-{(3,3,3-trifluoropropyl)-[3-(5-fluoro-1H-indol-3-yl)propyl]amino}-8-methoxychromane-5-methylcarboxamide;
(3R)-3-{(3,3,3-trifluoropropyl)-[3-(5-fluoro-1H-indol-3-yl)propyl]amino}-8-methoxychromane-5-methylcarboxamide;
(+)-3-{(3,3,3-trifluoropropyl)-[3-(5-fluoro-1H-indol-3-yl)propyl]amino}-8-methoxychromane-5-methylcarboxamide;
(−)-3-{(3,3,3-trifluoropropyl)-[3-(5-fluoro-1H-indol-3-yl)propyl]amino}-8-methoxychromane-5-methylcarboxamide;
3-{cyclobutyl[3-(5-fluoro-1H-indol-3-yl)propyl]amino}-8-(ethylthio)chromane-5-carboxamide;
(3R)-3-{cyclobutyl[3-(5-fluoro-1H-indol-3-yl)propyl]amino}-8-(ethylthio)chromane-5-carboxamide; (+)-3-{cyclobutyl[3-(5-fluoro-1H-indol-3-yl)propyl]amino}-8-(ethylthio)chromane-5-carboxamide; and
(−)-3-{cyclobutyl[3-(5-fluoro-1H-indol-3-yl)propyl]amino}-8-(ethylthio)chromane-5-carboxamide;
   or pharmaceutically acceptable salts or polymorphs thereof.

Both the R and S stereoisomers of the compounds and pharmaceutically acceptable salts of Formulae I, II or III, as well as mixtures of the R and S stereoisomers, are within the scope of the embodiments described herein. Throughout this application, where the absolute configuration of the compounds and pharmaceutically acceptable salts of Formulae I, II or III, is not indicated, is intended to embrace the individual R and S enantiomers as well as mixtures of the two.

R and S stereoisomers at the carbon alpha or beta from the basic nitrogen at position 3 of the chormane group of Formulae I, II or III are also within the scope of the embodiments described herein. Throughout this application, where the absolute configuration at the above two positions is not indicated, is intended to embrace the individual R and S enantiomers. In some embodiments, the compound of Formula I is the R enantiomer of one of the above exemplary compounds.

In some embodiments, the stereoisomer is substantially free of the corresponding enantiomer. Thus, an enantiomer substantially free of the corresponding enantiomer refers to a compound which is isolated or separated via separation techniques or prepared free of the corresponding enantiomer. In some embodiments, stereoisomers are isolated from racemic mixtures by any method known to those skilled in the art, including high performance liquid chromatography (e.g., using a chiral column) and the formation and crystallization of chiral salts or prepared by methods described herein. See, for example, Jacques, et al., Tetrahedron 33:2725 (1977); Eliel, E. L., Stereochemistry of Carbon Combounds (McGraw-Hill, NY, 1962); Wilen, S. H., Tables of Resolving Agents and Optical Resolutions P. 268 (E. L. Eliel, Ed., Univ. Notre Dame Press, Notre Dame, Ind. 1972).

In some embodiments, the compounds and pharmaceutically acceptable salts described herein exist as tautomers. Such tautomers can be transient or isolatable as a stable product. These tautomers are within the scope of the compounds and pharmaceutically acceptable salts described herein.

In certain embodiments, the present invention is directed to prodrugs of compounds of Formulae I, II, or III. Various forms of prodrugs are known in the art, for example, as discussed in, for example, Bundgaard, (ed.), Design of Prodrugs, Elsevier (1985); Widder, et al. (ed.), Methods in Enzymology, vol. 4, Academic Press (1985); Krogsgaard-Larsen, et al. (ed.), “Design and Application of Prodrugs”, Textbook of Drug Design and Development, Chapter 5, 113-191 (1991), Bundgaard, et al., Journal of Drug Deliver reviews, 8:1-38 (1992), Bundgaard, J. of Pharmaceutical Sciences, 77:285 et seq. (1988); and Higuchi and Stella (eds.) Prodrugs as Novel Drug Delivery Systems, American Chemical Society (1975), each of which is incorporated by reference in its entirety.

In certain embodiments, the present invention provides a method of synthesizing a compound comprising:

(a) reacting a compound of Formula 1:

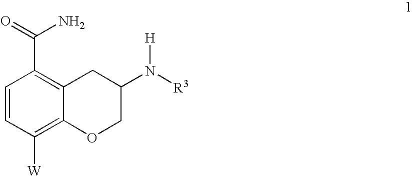

wherein

W is halogen; and

R³ is —(CH₂)ₙ—R⁴;

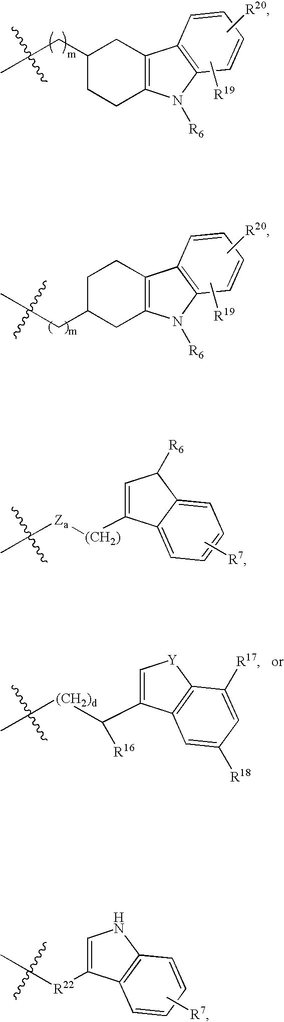

n is an integer of 2 to 4; and R⁴ is A, B, C, D, E, G, J, K, L, P, U, AA or BB, provided that when n is an integer of 2, then R⁴ is A, B, C, D, E, G, J, K, L, U, AA or BB; and further provided that when n is an integer of 3 or 4, then R⁴ is A, B, C, D, K, L, P, U, AA or BB;

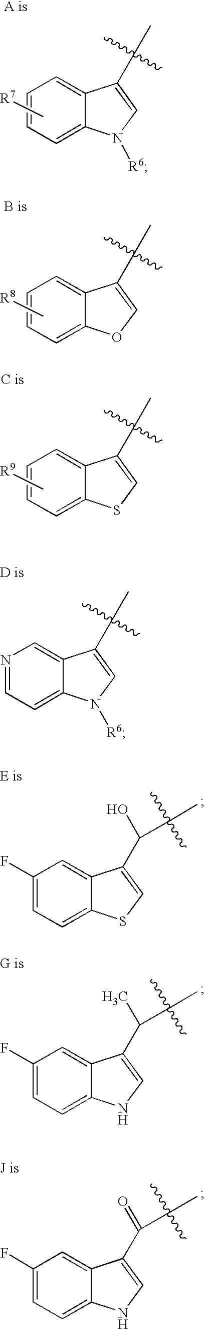

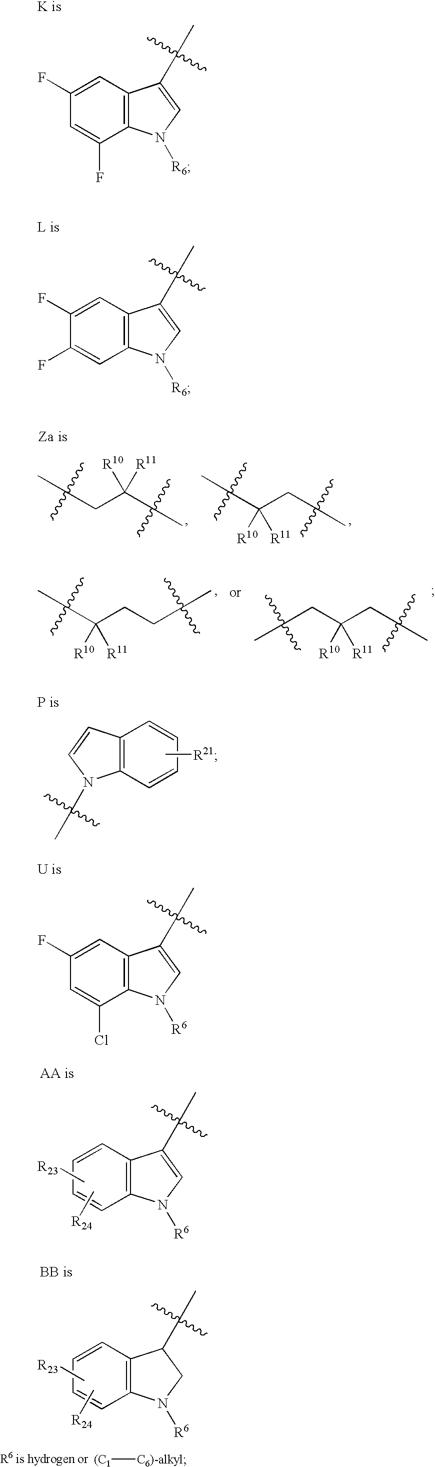

R⁷ is hydrogen, fluoro, chloro, cyano, or alkoxy;

R⁵ is hydrogen, halo, (C₁-C₃)-alkoxy or (C₁-C₃)-alkyl;

R⁹ is hydrogen, halo, (C₁-C₃)-alkoxy or (C₁-C₃)-alkyl;

R¹⁰ is hydrogen or methyl;

R¹¹ is methyl;

R¹⁹ and R²⁰ are independently hydrogen, fluoro, chloro, cyano, or (C₁-C₆)-alkyl;

R²¹ is hydrogen or fluoro;

R²² is a 3- to 7-membered saturated carbocyclic ring;

R²³ and R²⁴ are independently hydrogen, halogen, cyano, or (C₁-C₆)-alkyl;

m is an integer of 1 or 2;

Y is O, S, or NH;

provided that when Y is O, then R¹⁶ is hydrogen; R¹⁷ is hydrogen or OCH₃; R¹⁸ is hydrogen; and d is an integer of 2 or 3;

further provided that when Y is S, then R¹⁶ is hydrogen or hydroxyl; R¹⁷ is hydrogen; R¹⁸ is hydrogen or fluoro; and d is an integer of 2;

further provided that when Y is NH, then R¹⁶ is keto (═O) or methyl; R¹⁷ is hydrogen; R¹⁸ is fluoro; and d is an integer of 2;

with an alkyl alcohol salt of Formula 1a:

R¹²O⁻Z⁺ (1a)

wherein R¹² is (C₁-C₆)-alkyl; and

Z is a pharmaceutically acceptable counter ion;

under conditions effective to produce a compound of Formula 2:

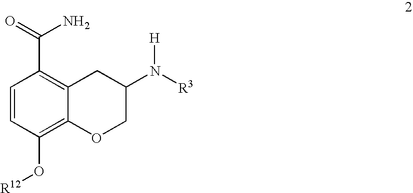

(b) optionally converting the compound of Formula 2 to a compound of Formula 3:

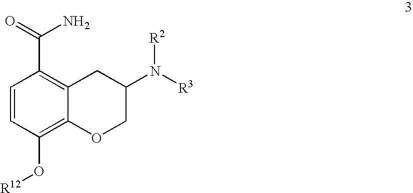

wherein R² is (C₁-C₄)-alkyl, (C₃-C₆)-cycloalkyl, cycloalkylalkyl, aralkyl, or haloalkyl;

under conditions effective to bring about reductive amination at the basic nitrogen of the compound of Formula 2; and

(c) further optionally reacting the compound of Formula 3 under conditions effective to remove the R¹² group, thereby providing a compound of Formula 4:

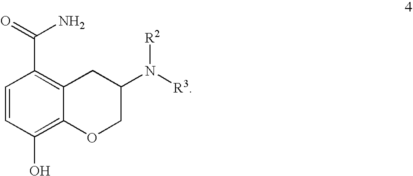

In certain embodiments, the compound of Formula 2, 3 or 4 further reacts under conditions effective to bring about hydrolysis of the amino group to provide a compound of Formula 8:

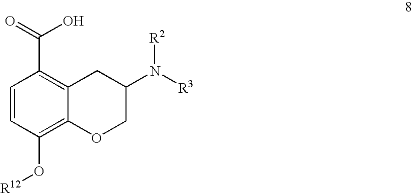

wherein R² is hydrogen, (C₁-C₄)-alkyl, (C₃-C₆)-cycloalkyl, cycloalkylalkyl, aralkyl, or haloalkyl; R³ is as defined claim 16; and R¹² is hydrogen or (C₁-C₆)-alkyl.

In certain other embodiments, the compound of Formula 8 further reacts with an alkylamine of Formula 8a:

R¹NH₂ (8a)

wherein R¹ is hydrogen or (C₁-C₃)-alkyl;

under conditions effective to provide a compound of Formula Ia:

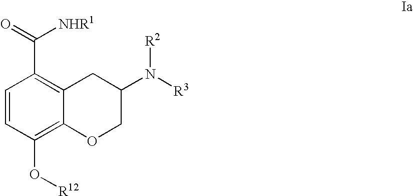

wherein R¹, R², R³, and R¹² are as defined hereinabove.

In certain embodiments, the method further comprises the following steps:

(a) reacting the compound of Formula 4 under conditions effective to produce a compound of Formula 5

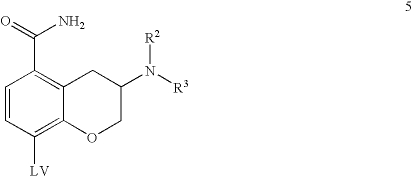

wherein LV is a precursor for a palladium catalyzed reaction;

(b) reacting a compound of Formula 5 under conditions effective to product a compound of Formula 7:

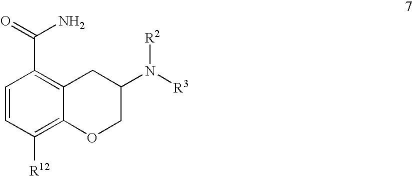

wherein R¹² is (C₁-C₆)-alkyl;

(c) reacting the compound of Formula 7 under conditions effective to bring about hydrolysis of the amino group to provide a compound of Formula 8:

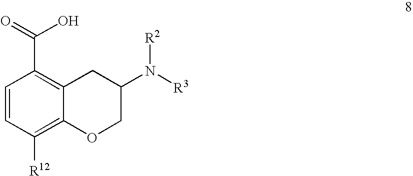

(d) reacting the compound of Formula 8 with an alkylamine of formula 8a:

R¹NH₂ (8a)

wherein R¹ is hydrogen or (C₁-C₃)-alkyl;

under conditions effective to provide a compound of Formula Ib:

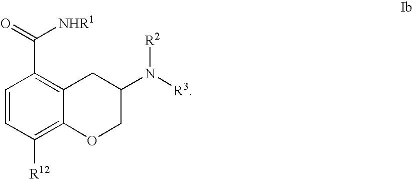

In certain embodiments, the present invention provides a method of synthesizing a compound comprising:

(a) reacting a compound of Formula 1:

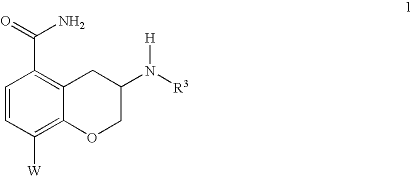

wherein

W is halogen; and

R³ is —(CH₂)ₙ—R⁴,

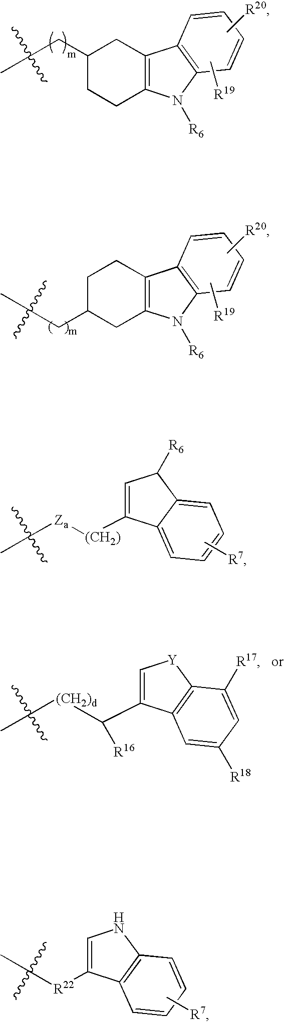

n is an integer of 2 to 4; and R⁴ is A, B, C, D, E, G, J, K, L, P, U, AA or BB, provided that when n is an integer of 2, then R⁴ is A, B, C, D, E, G, J, K, L, U, AA or BB; and further provided that when n is an integer of 3 or 4, then R⁴ is A, B, C, D, K, L, P, U, AA or BB;

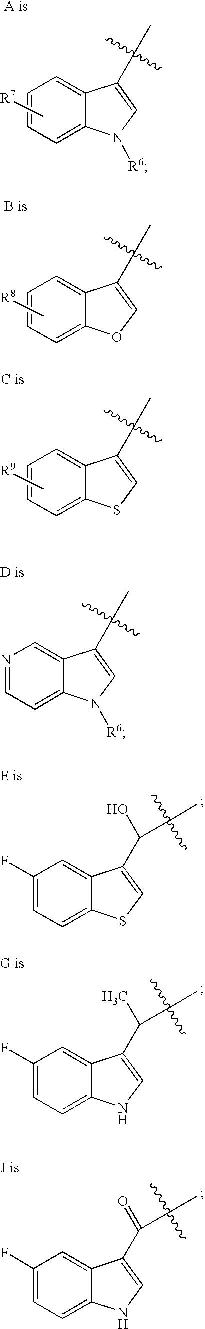

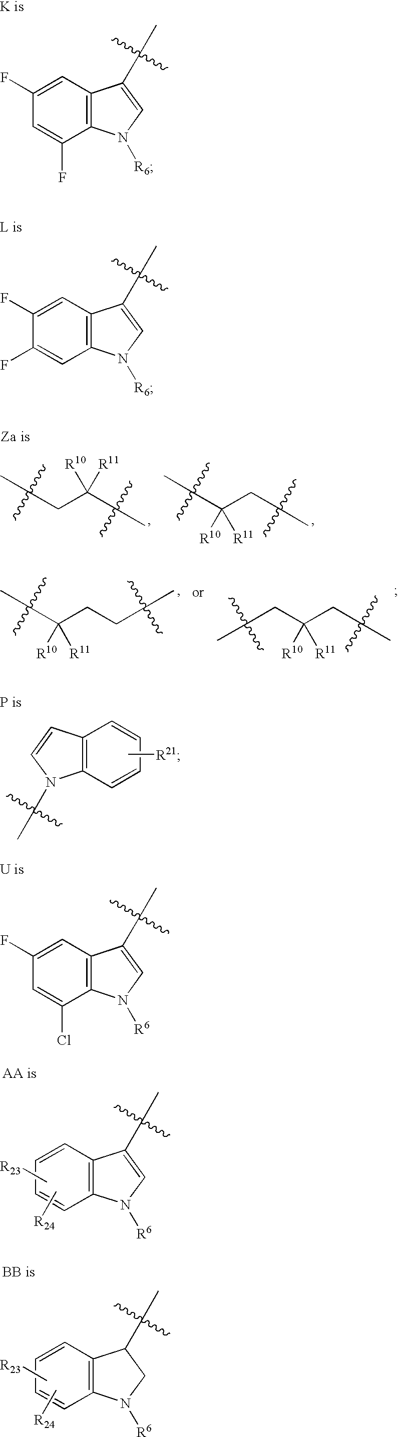

R⁶ is hydrogen or (C₁-C₆)-alkyl;

R⁷ is hydrogen, fluoro, chloro, cyano, or alkoxy;

R⁸ is hydrogen, halo, (C₁-C₃)-alkoxy or (C₁-C₃)-alkyl;

R⁹ is hydrogen, halo, (C₁-C₃)-alkoxy or (C₁-C₃)-alkyl;

R¹⁰ is hydrogen or methyl;

R¹¹ is methyl;

R¹⁹ and R²⁰ are independently hydrogen, fluoro, chloro, cyano, or (C₁-C₆)-alkyl;

R²¹ is hydrogen or fluoro;

R²² is a 3- to 7-membered saturated carbocyclic ring;

R²³ and R²⁴ are independently hydrogen, halogen, cyano, or (C₁-C₆)-alkyl;

m is an integer of 1 or 2;

Y is O, S, or NH;

provided that when Y is O, then R¹⁶ is hydrogen; R¹⁷ is hydrogen or OCH₃; R¹⁸ is hydrogen; and d is an integer of 2 or 3;

further provided that when Y is S, then R¹⁶ is hydrogen or hydroxyl; R¹⁷ is hydrogen;

R¹⁸ is hydrogen or fluoro; and d is an integer of 2;

further provided that when Y is NH, then R¹⁶ is keto (═O) or methyl; R¹⁷ is hydrogen;

R¹⁸ is fluoro; and d is an integer of 2;

with an alkylthio salt of Formula Ia or an amine salt of Formula Ib:

R¹²S⁻Z⁺ (1a)

or R¹³R¹⁴N⁻Z⁺ (1b)

wherein R¹², R¹³, and R¹⁴ are each independently hydrogen or (C₁-C₆)-alkyl, and Z is

a pharmaceutically acceptable counter ion;
under conditions effective to produce a compound of Formula 6:

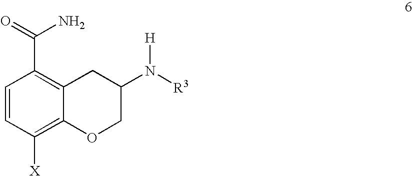

wherein X is —SR¹² or —NR¹³R¹⁴;

(b) reacting the compound of Formula 6 with an alkylaldehyde of Formula 6a or a ketone of Formula 6b:

R²C(O)H (6a);

R²C(O)R² (6b);

wherein R² is hydrogen, (C₁-C₄)-alkyl, (C₃-C₆)-cycloalkyl, cycloalkylalkyl, aralkyl,

or haloalkyl;
under conditions effective to bring about reductive alkylation at the basic nitrogen to product a compound of Formula 7:

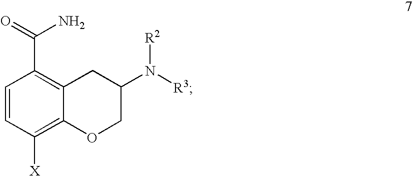

(c) reacting the compound of Formula 7 under conditions effective to bring about hydrolysis of the amino group to provide a compound of Formula 8:

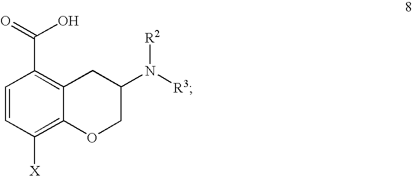

(d) reacting the compound of Formula 8 with an alkylamine of Formula 8a:

R¹NH₂ (8a)

wherein R¹ is hydrogen or (C₁-C₃)-alkyl;

under conditions effective to provide a compound of Formula Ic:

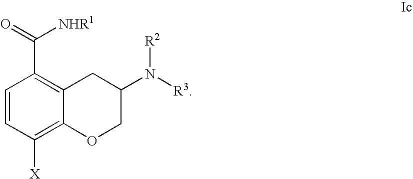

In certain embodiments, the present invention provides a method of synthesizing a compound comprising:

(a) reacting a compound of Formula 10:

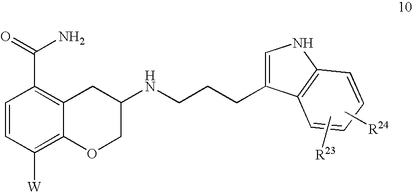

wherein W is halogen:

R²³ and R²⁴ are each independently hydrogen, —F, —Cl, —Br, —I, —CN, or (C₁-C₆)-alkyl;

with an alkyl alcohol salt of Formula Ia:

R¹²O⁻Z⁺ (1a)

wherein R¹² is (C₁-C₆)-alkyl and Z is a pharmaceutically acceptable counter ion; under conditions effective to produce a compound of Formula 11:

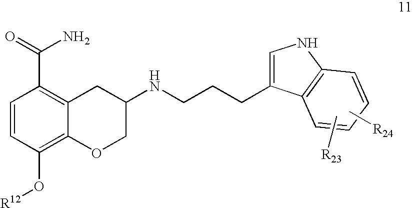

(b) converting the compound of Formula 11 to a compound of Formula 12:

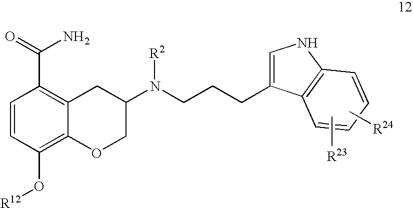

wherein R² is hydrogen, (C₁-C₄)-alkyl, (C₃-C₆)-cycloalkyl, cycloalkylalkyl, aralkyl, or haloalkyl;

under conditions effective to bring about reductive amination at the basic nitrogen of the compound of Formula 11;

(c) optionally reacting the compound of Formula 12 under conditions effective to remove the R¹² group, thereby providing a compound of Formula 13:

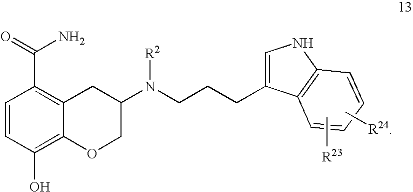

In certain other embodiments, the method further comprises the following steps:
(a) reacting a compound of Formula 12 or 13 under conditions effective to bring about hydrolysis of the amino group to provide a compound of Formula 16,

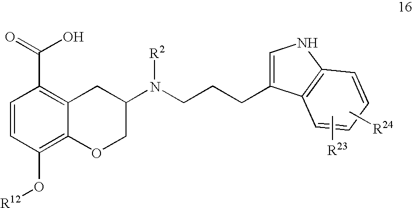

wherein R¹² is hydrogen or (C₁-C₆)-alkyl; and
(b) reacting the compound of Formula 16 with an alkylamine of Formula 16a:

R¹NH₂ (16a)

wherein R¹ is hydrogen or (C₁-C₃)-alkyl;

under conditions effective to provide a compound of Formula Ia:

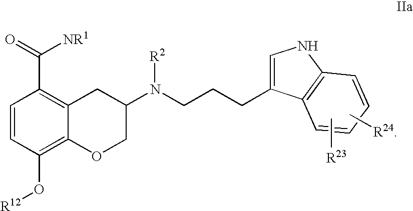

In certain embodiments, the method further comprises reacting the compound of Formula IIa under conditions effective to bring about reduction of the indol to produce a compound of Formula IIIa:

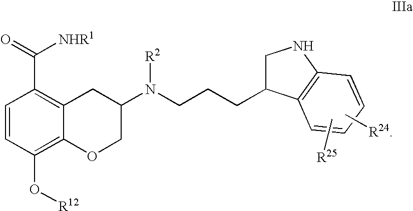

In certain embodiments, the present invention provides a method of synthesizing a compound comprising:
(a) reacting a compound of Formula 10:

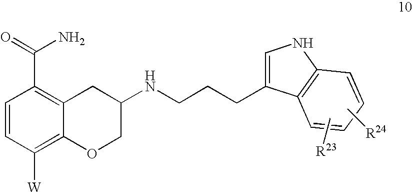

wherein W is halogen:

R²³ and R²⁴ are each independently hydrogen, —F, —Cl, —Br, —I, —CN, or (C₁-C₆)-alkyl; with an alkyl alcohol salt of Formula 10a:

R¹²O⁻Z⁺ (10a);

wherein R¹² is hydrogen or (C₁-C₆)-alkyl and Z is a pharmaceutically acceptable counter ion;

under conditions effective to produce a compound of Formula 11:

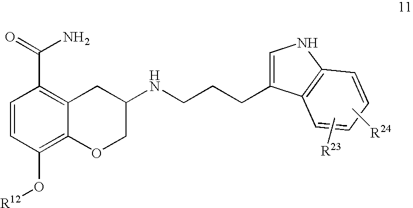

(b) converting the compound of Formula 11 to a compound of Formula 12:

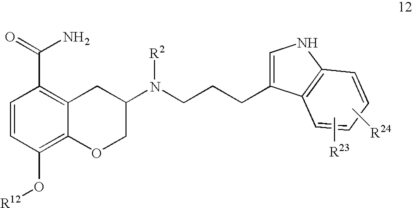

wherein R² is hydrogen, (C₁-C₄)-alkyl, (C₃-C₆)-cycloalkyl, cycloalkylalkyl, aralkyl, or haloalkyl;

under conditions effective to bring about reductive amination at the basic nitrogen of the compound of Formula 11;
(c) reacting the compound of Formula 12 under conditions effective to remove the R¹² group, thereby providing a compound of Formula 13:

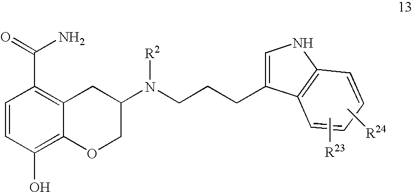

(d) reacting the compound of Formula 13 under conditions effective to produce a compound of Formula 14

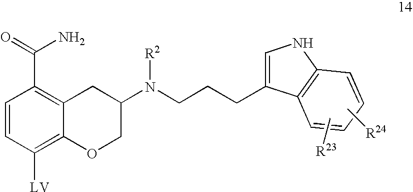

wherein LV is a precursor for a palladium catalyzed reaction; and
(e) reacting a compound of Formula 14 under conditions effective to product a compound of Formula 15:

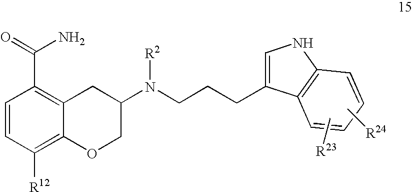

wherein R¹² is (C₁-C₆)-alkyl.

In certain other embodiments, the method further comprises the following steps:

(a) reacting the compound of Formula 15 under conditions effective to bring about hydrolysis of the amino group to provide a compound of Formula 16:

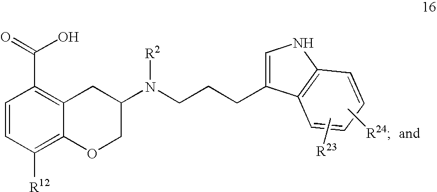

(b) reacting the compound of Formula 16 with an alkylamine of Formula 16a:

R¹NH₂ (16a)

wherein R¹ is hydrogen or (C₁-C₃)-alkyl;

under conditions effective to provide a compound of Formula IIb:

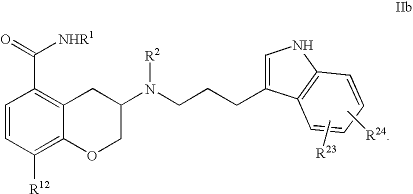

In some cases, the method further comprises reacting the compound of Formula IIb under conditions effective to bring about reduction of the indol to produce a compound of Formula IIIb:

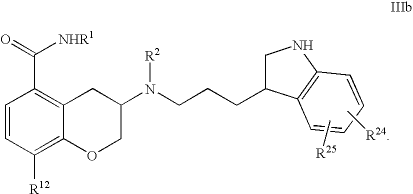

In certain embodiments, the present invention provides a method of synthesizing a compound comprising:

(a) reacting a compound of Formula 10:

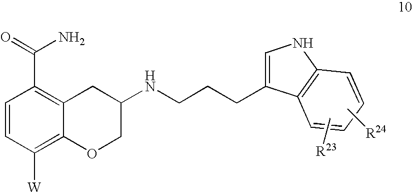

wherein W is halogen:

R²³ and R²⁴ are each independently hydrogen, —F, —Cl, —Br, —I, —CN, or (C₁-C₆)-alkyl; with an alkylthio salt of Formula 10a or an amine salt of Formula 10b:

R¹²S⁻Z⁺ (10a)

or R¹³R¹⁴N⁻Z⁺ (10b)

wherein R¹², R¹³, and R¹⁴ are each independently hydrogen or (C₁-C₆)-alkyl and Z is a pharmaceutically acceptable counter ion;

under conditions effective to produce a compound of Formula 18:

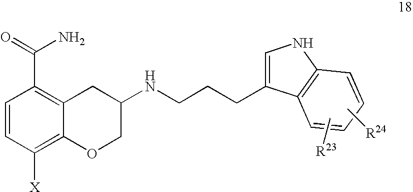

wherein X is —SR¹² or —NR¹³R¹⁴;

(b) converting the compound of Formula 18 to a compound of Formula 15:

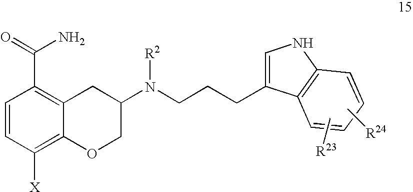

wherein R² is hydrogen, (C₁-C₄)-alkyl, (C₃-C₆)-cycloalkyl, cycloalkylalkyl, aralkyl, or haloalkyl;

under conditions effective to bring about reductive alkylation at the basic nitrogen of Formula 18.

In certain other embodiments, the method further comprises the following steps:

(a) reacting the compound of Formula 15 under conditions effective to bring about hydrolysis of the amino group to provide a compound of Formula 16:

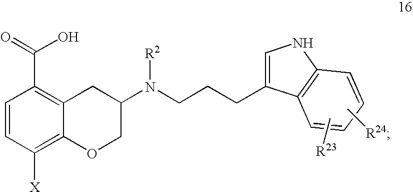

(b) reacting the compound of Formula 16 with an alkylamine of Formula 16a:

R¹NH₂ (16a)

wherein R¹ is hydrogen or (C₁-C₃)-alkyl;

under conditions effective to provide a compound of Formula IIc:

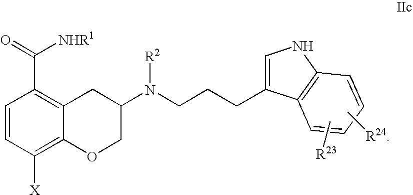

In certain embodiments, the method further comprises reacting the compound of Formula IIc under conditions effective to bring about reduction of the indol to produce a compound of Formula IIIc:

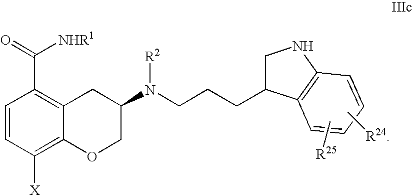

In the synthetic methods described herein, in certain embodiments, W is —F, —Cl, or —Br. In certain other embodiments, R¹² is (C₁-C₃)-alkyl. In yet other embodiments, Z is Na⁺, K⁺, or Li⁺. In yet other embodiments, LV is triflate.

The compounds and pharmaceutically acceptable salts described herein can be prepared using a variety of methods starting from commercially available compounds, known compounds, or compounds prepared by known methods. General synthetic routes to many of the compounds described herein are included in the following schemes. The methods for making some intermediates and precursor compounds are described in WO 2005/011291, which is incorporated in its entirety by reference. It is understood by those skilled in the art that protection and deprotection steps not shown in the Schemes may be required for these syntheses, and that the order of steps may be changed to accommodate functionality in the target molecule. The need for protection and deprotection, and the selection of appropriate protecting groups can be found, for example, in Greene and Wuts, Protecting Groups in Organic Synthesis, Third Edition, John Wiley & Sons (1999), which is incorporated by reference in its entirety.

In the schemes described herein, appropriate polar solvents solvents include, but are not limited to, dimethyl sulfoxide, dimethylformamide, tetrahydrofuran, methanol and ethanol. Suitable acid binding agents include, but are not limited to, organic tertiary bases, such as, for example, triethylamine, triethanolamine, 1,8-diazabicyclo[5.4.0]undec-7-ene (DBU), and diisopropylethylamine (DIPEA); and alkaline metal carbonates, such as, for example, potassium carbonate and sodium carbonates. Suitable reducing agents include, but are not limited to, sodium cyanoborohydride and sodium triacetoxyborohydride.

Scheme 1 illustrates one process for making compound of Formula I, where R¹, R², R³ and X are as defined herein. As shown in Scheme 1, to prepare compounds of Formula I where X is —OH, compound 1 containing a halogen, W (e.g., fluorine, chlorine, bromine), is reacted with a salt of a lower alkyl alcohol (e.g., ZOR¹², where Z is an appropriate counter ion, including, but not limited to, Na, K and Li; non-limiting examples of ZOR¹² include sodium methoxide, sodium ethoxide, sodium propoxide, etc.) under conditions effective to produce compound 2. For example, in some embodiments, the reaction is performed in the presence of the corresponding alcohol and the salt and is heated to reflux for 24 hours. Compound 2 is then reacted under conditions effective to bring about reductive amination at the basic nitrogen of compound 2 to afford compound 3. For example, in some embodiments, compound 2 is reacted with an aldehyde or ketone (e.g., R′C(═O)H or R″C(═O)R′″, wherein R″ and R′″ may be linked to form a cyclic ketone and wherein the total number of carbons in R′C(═O)H or R′C(═O)R″ equals to that in R²) to introduce the appropriate alkyl chain R² group at the basic nitrogen. In some embodiments, the reaction proceeds in sodium cyanoborohydride or sodium triacetoxyborohydride. Compound 3 is then reacted under conditions effective to remove the alkyl group (e.g., R¹²), thereby producing the hydroxychormane analog, compound 4. For example, in some embodiments, the reaction proceeds with BBr₃ in CH₂Cl₂. In one embodiment, where R¹ is other than hydrogen, compound 4 is reacted under conditions effective to bring about the hydrolysis of the amino group, thereby producing compound 8. Compound 8 is reacted with an alkyl amine (R¹NH₂) under conditions effective to produce a compound of Formula I.

To prepare compounds of Formula I where X is alkoxy, compound 1 containing a halogen, W (e.g., fluorine, chlorine, bromine), is reacted with a salt of a lower alkyl alcohol (e.g., ZOR¹², where Z is an appropriate counter ion, including but not limited to, Na, K and Li; non-limiting examples of ZOR¹² include sodium methoxide, sodium ethoxide, sodium propoxide, etc.) as described above to produce compound 2. Compound 2 is then reacted as described above to afford compound 3. Compound 3 is reacted under conditions effective to bring about hydrolysis of the amino group to provide the carboxylic acid analog, compound 8. Compound 8 is reacted with an alkylamine containing the appropriate R¹ group (e.g., R¹NH₂, including but not limited to methylamine, ethylamine, propylamine, or isopropylamine) under conditions effective to provide compounds of Formula I, where X is alkoxy.

To prepare compounds of Formula I where X is alkyl, compound 1 containing a halogen, W (e.g., fluorine, chlorine, bromine), is reacted with a salt of a lower alkyl alcohol (e.g., ZOR¹², where Z is an appropriate counter ion, including, but not limited to, Na, K and Li; non-limiting examples of ZOR¹² include sodium methoxide, sodium ethoxide, sodium propoxide, etc.) as described above to produce compound 2. Compound 2 is then reacted as described above to afford compound 3. Compound 3 is then reacted as described above to produce the hydroxychormane analog, compound 4. Compound 4 is then reacted under appropriate conditions to convert compound 4 to a precursor compound for a palladium catalyzed reaction (e.g., by converting the hydroxyl group to triflate, i.e., trifluoromethanesulfonate), affording compound 5. Compound 5 is then reacted under conditions appropriate to produce compound 7. For example, in some embodiments, compound 5 is subjected to a coupling reaction (e.g., Stille coupling) with an appropriate reactant (e.g., (R¹²)₄M, where M is a metal including but not limited to tin, such as tetraalkyltin) to provide the alkylchormane analog, compound 7. Compound 7 is then reacted under conditions effective to bring about hydrolysis of the amino group to provide the carboxylic acid analog, compound 8. Compound 8 is reacted with an alkylamine containing the appropriate R¹ group (e.g., R¹NH₂, including but not limited to methylamine, ethylamine, propylamine, or isopropylamine) under conditions effective to provide compounds of Formula I.

To prepare compounds of Formula I where X is alkylthio or alkylamino, compound 1 is reacted with an alkylthio salt (e.g., R¹²SZ, where Z is an appropriate counter ion, including but not limited to, sodium, potassium, or lithium) or the salt of an amine (e.g., R¹³R¹⁴NZ, where Z is an appropriate counter ion, including but not limited to, sodium, potassium, or lithium) under conditions effective to provide compound 6, where X is alkylthio or alkylamino. Compound 6 is reated with an appropriate aldehyde or ketone (e.g., R′C(═O)H or R″C(═O)R′″, wherein R″ and R′″ may be linked to form a cyclic ketone and wherein the total number of carbons in R′C(═O)H or R′C(═O)R″ equals to that in R²) to bring about reductive alkylation at the basic nitrogen to introduce the appropriate alkyl chain R² group, thereby producing the tertiary amine, compound 7. Compound 7 is then reacted under conditions effective to bring about hydrolysis of the amino group to provide the carboxylic acid analog, compound 8. Compound 8 is reacted with an alkylamine containing the appropriate R¹ group (e.g., R¹NH₂, including but not limited to methylamine, diethyleamine, ethylamine, diethylamine, propylamine, di propylamine, isopropylamine or diisopropylamine) under conditions effective to provide compounds of Formula I.

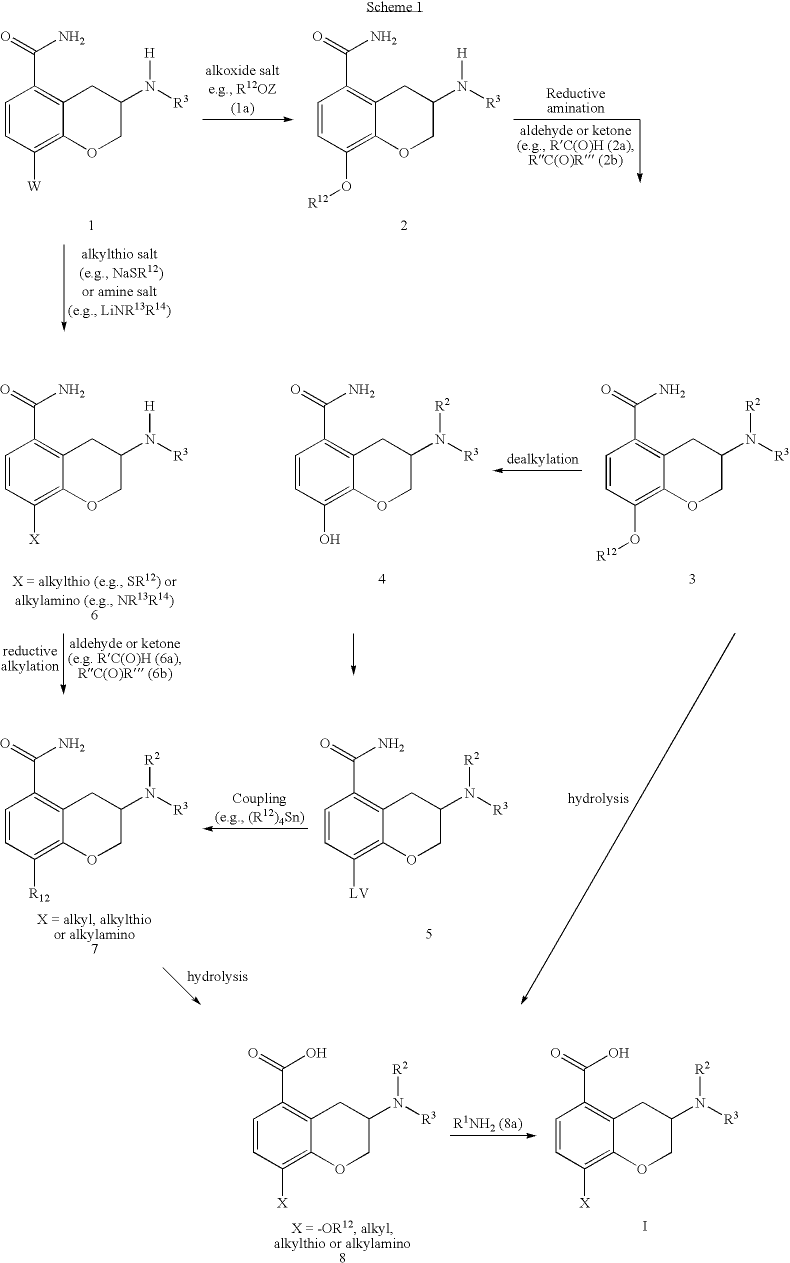

Scheme 2 illustrates an exemplary process for the preparation of compounds of Formula II or III, where R¹, R², X, Y, and Z are as defined herein.

As shown in Scheme 2, to prepare compounds of Formula II where X is —OH, compound 10 containing a halogen, W (e.g., fluorine, chlorine, bromine), is reacted with a salt of a lower alkyl alcohol (e.g., ZOR¹², where Z is an appropriate counter ion, including but not limited to, Na, K and Li; non-limiting examples of ZOR¹² include sodium methoxide, sodium ethoxide, sodium propoxide, etc.) under conditions effective to produce compound 11. For example, in some embodiments, the reaction is performed in the presence of the corresponding alcohol and the salt and is heated to reflux for 24 hours. Compound 11 is then reacted under conditions effective to bring about reductive amination at the basic nitrogen of compound 11 to afford compound 12. For example, in some embodiments, compound 11 is reacted with an aldehyde or ketone (e.g., R′C(═O)H or R″C(═O)R′″, wherein R″ and R′″ may be linked to form a cyclic ketone and wherein the total number of carbons in R′C(═O)H or R″C(═O)R′″ equals to that in R²) to introduce the appropriate alkyl chain R² group at the basic nitrogen. In some embodiments, the reaction proceeds in sodium cyanoborohydride or sodium triacetoxyborohydride. Compound 12 is then reacted under conditions effective to remove the alkyl group (e.g., R¹²), thereby producing the hydroxychormane analog, compound 13. For example, in some embodiments, the reaction proceeds with BBr₃ in CH₂Cl₂. In one embodiment, where R¹² is other than hydrogen, compound 12 is reacted under conditions effective to bring about the hydrolysis of the amino group, thereby producing compound 16. Compound 16 is reacted with an alkyl amine (R¹NH₂) under conditions effective to produce a compound of Formula II. Moreover, alternatively, to produce an indoline analog, compound 12 or II is reacted under conditions effective to bring about reduction of the indol group to produce compound 17 or III with an indoline group.

To prepare compounds of Formula II and Formula III where X is alkoxy, compound 10 containing a halogen, W (e.g., fluorine, chlorine, bromine), is reacted with a salt of a lower alkyl alcohol (e.g., ZOR¹², where Z is an appropriate counter ion, including but not limited to, Na, K and Li; non-limiting examples of ZOR¹² include sodium methoxide, sodium ethoxide, sodium propoxide, etc.) as described above to produce compound 11. Compound 11 is then reacted as described above to afford compound 12. Compound 12 is reacted as described above under conditions effective to bring about hydrolysis of the amino group to provide the carboxylic acid analog, compound 16. Compound 16 is reacted with an alkylamine containing the appropriate R¹ group (e.g., R¹NH₂, including but not limited to methylamine, dimethyleamine, ethylamine, diethylamine, propylamine, dipropylamine, isopropylamine or diisopropylamine) under conditions effective to provide compounds of Formula II, where X is alkoxy (IIa). The corresponding indoline analog compounds of Formula III are prepared by reduction of compound II, for example with sodium borohydride in TFA. Alternatively, to produce the indoline analog where R¹ is hydrogen, compound 12 is reacted under conditions effective to bring about reduction of the indol to produce compound 17.

To prepare compounds of Formula I where X is alkyl, compound 10 containing a halogen, W (e.g., fluorine, chlorine, bromine), is reacted with a salt of a lower alkyl alcohol (e.g., ZOR¹², where Z is an appropriate counter ion, including but not limited to, Na, K and Li; non-limiting examples of ZOR¹² include sodium methoxide, sodium ethoxide, sodium propoxide, etc.) as described above to produce compound 11. Compound 11 is then reacted as described above to afford compound 12. Compound 12 is then reacted as described above to produce the hydroxychormane analog, compound 13. Compound 13 is then reacted under appropriate conditions to convert compound 13 to a precursor compound for a palladium catalyzed reaction (e.g., by converting the hydroxyl group of compound 13 to triflate), affording compound 14. Compound 14 is then reacted under conditions appropriate to produce compound 15. For example, in some embodiments, compound 14 is subjected to a coupling coupling reaction (e.g., Stille coupling) with an appropriate reactant (e.g., (R¹²)₄M, where M is a metal including but not limited to tin, such as tetraalkyltin) to provide the alkylchormane analog, compound 15. Compound 15 is then reacted under conditions effective to bring about hydrolysis of the amino group to provide the carboxylic acid analog, compound 16. Compound 16 is reacted with an alkylamine containing the appropriate R¹ group (e.g., R¹NH₂, including but not limited to methylamine, dimethyleamine, ethylamine, diethylamine, propylamine, dipropylamine, isopropylamine or diisopropylamine) under conditions effective to provide compounds of Formula II, where X is alkyl (IIb). The corresponding indoline analog compounds of Formula III are prepared by reduction of compound II, for example with sodium borohydride in TFA.

To prepare compounds of Formulae II or III where X is alkylthio or alkylamino, compound 10 is reacted with an alkylthio salt (e.g., a sodium salt) (e.g., R¹²SZ, where Z is an appropriate counter ion, including but not limited to, sodium, potassium, or lithium) or the salt of an amine (e.g., R¹³R¹⁴NZ, where Z is an appropriate counter ion, including but not limited to, sodium, potassium, or lithium) as described above to provide compound 18, where X is alkylthio or alkylamino. Compound 18 is reacted with an appropriate aldehyde or ketone (e.g., R′C(═O)H or R″C(═O)R′″, wherein R″ and R′″ may be linked to form a cyclic ketone and wherein the total number of carbons in R′C(═O)H or R″C(═O)R′″ equals to that in R²) to bring about reductive alkylation at the basic nitrogen to produce the tertiary amine, compound 15. Compound 15 is then reacted under conditions effective to bring about hydrolysis of the amino group to provide the carboxylic acid analog, compound 16. Compound 16 is reacted with an alkylamine containing the appropriate R¹ group (e.g., R¹NH₂, including but not limited to methylamine, dimethyleamine, ethylamine, diethylamine, propylamine, dipropylamine, isopropylamine or diisopropylamine) under conditions effective to provide compounds of Formula II, where X is alkylthio or alkylamino (IIc). The corresponding indoline analog compounds of Formula III are prepared by reduction of compound II, for example with sodium borohydride in TFA.

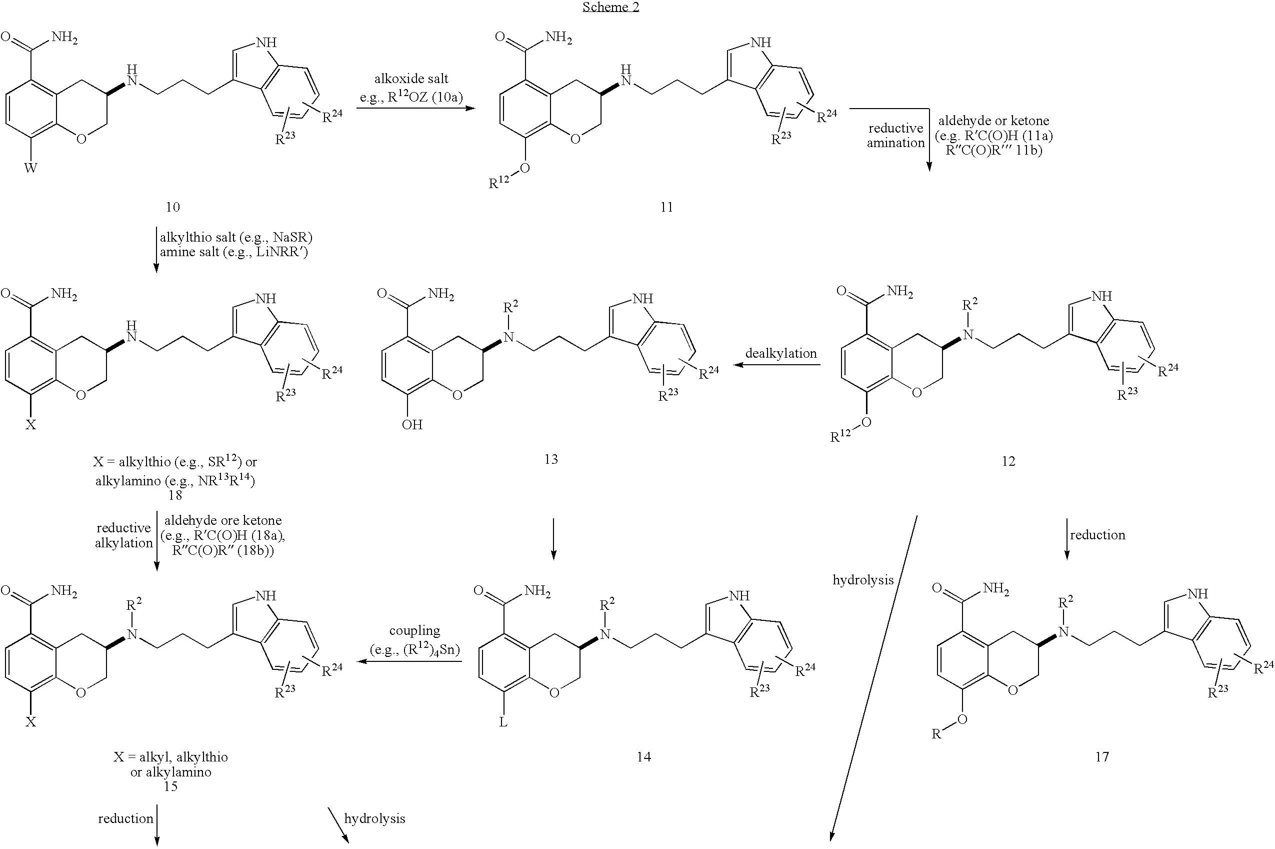

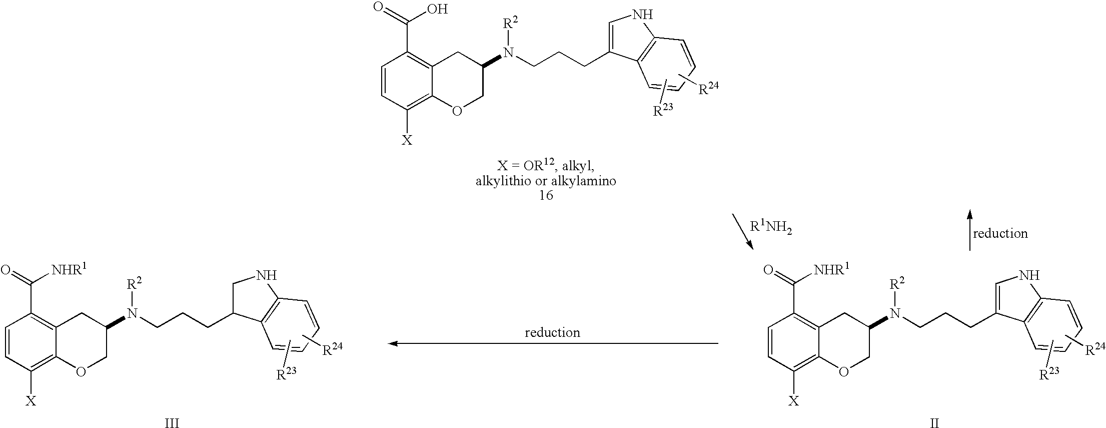

Synthetic intermediates useful for preparing the compounds described herein may be prepared according to methods known to those in the art or methods published elsewhere. In some embodiments, the starting materials and reactants described herein described herein are either available from commercial sources or may be prepared according to the methods described herein, or according to methods known to those of skill in the art.

In some embodiments, the compounds described herein are further reacted to form a salt through an acid addition process. In one nonlimiting example, one or more equivalents of an acid are reacted with the free base of a compound described herein to form an acid addition salt. Exemplary salts include, without limitation, mono-, di-, tri, and tetra-acid salts.

Schemes 1 and 2 illustrate the synthetic metholodolgy used to prepare particular compounds described herein. One of skill in the art will recognize that Schemes 1 and 2 can be adapted to produce other compounds according to one or more embodiments described herein and that other methods may be used to produce the compounds described herein.

In certain embodiments, the present invention provides pharmaceutical composition comprising a compound described herein or a pharmaceutically acceptable salt thereof and one or more pharmaceutically acceptable carriers. In some embodiments, the compounds or pharmaceutically acceptable salts of the compounds described herein are a component of a composition that includes one or more pharmaceutically acceptable vehicles, carriers, excipients, or diluents. For example, in some embodiments, solid carriers include one or more substances which may also act as flavoring agents, lubricants, solubilizers, suspending agents, fillers, glidants, compression aids, binders or tablet-disintegrating agents or encapsulating materials. Such pharmaceutical compositions can be prepared using a method including admixing the compound or pharmaceutically acceptable salt of the compound and a physiologically acceptable carrier, excipient, or diluent. Admixing can be accomplished using methods well known for admixing a compound or a pharmaceutically acceptable salt of a compound and a physiologically acceptable carrier, excipient, or diluent. Examples of such carriers, excipients, and diluents are well known to those skilled in the art and are prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), which is incorporated herein by reference in its entirety. Pharmaceutically acceptable carriers, excipients, and diluents are those that are compatible with the other ingredients in the formulation and biologically acceptable.

In addition, auxiliary, stabilizing, thickening, lubricating, and coloring agents can optionally be used. In one embodiment the physiologically acceptable excipients are sterile when administered to an animal. The physiologically acceptable excipient should be stable under the conditions of manufacture and storage and, as necessary, should be preserved against the contaminating action of microorganisms. The present compositions, if desired, also optionally contain minor amounts of wetting or emulsifying agents, or pH buffering agents. Other examples of suitable physiologically acceptable excipients are described in Remington's Pharmaceutical Sciences, pp. 1447-1676 (Alfonso R. Gennaro, ed., 19th ed. 1995).

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the composition is in the form of a capsule.

The compounds or pharmaceutically acceptable salts of the compounds described herein may be administered orally or parenterally, neat or in combination with conventional pharmaceutical carriers as described above. The compounds or pharmaceutically acceptable salts of the compounds described herein can also be administered by any convenient route, for example, orally, by infusion or bolus injection, by absorption through epithelial or mucocutaneous linings (e.g., oral, rectal, vaginal, and intestinal mucosa, etc.) and can be administered together with another therapeutic agent. Other methods of administration include, but are not limited to, intradermal, intramuscular, intraperitoneal, intravenous, subcutaneous, intranasal, epidural, sublingual, intracerebral, intravaginal, transdermal, rectal, by inhalation, or topical, particularly to the ears, nose, eyes, or skin. Administration can be systemic or local. Various known delivery systems, including encapsulation in liposomes, microparticles, microcapsules, and capsules, can be used. In some instances, administration will result of release of the compound or a pharmaceutically acceptable salt of the compound into the bloodstream. The mode of administration is left to the discretion of the practitioner.

In one embodiment, the compound or pharmaceutically acceptable salt of the compound is administered orally.

In another embodiment, the compound or pharmaceutically acceptable salt of the compound is administered intravenously.

In another embodiment, it may be desirable to administer the compounds or pharmaceutically acceptable salts of the compounds described herein locally. This can be achieved, for example, by local infusion during surgery, topical application, e.g., in conjunction with a wound dressing after surgery, by injection, by means of a catheter, by means of a suppository or edema, or by means of an implant, said implant being of a porous, non-porous, or gelatinous material, including membranes, such as sialastic membranes, or fibers.

In certain embodiments, it can be desirable to introduce the compounds or pharmaceutically acceptable salts of the compounds described herein into the central nervous system, circulatory system or gastrointestinal tract by any suitable route, including intraventricular, intrathecal injection, paraspinal injection, epidural injection, enema, and by injection adjacent to the peripheral nerve. Intraventricular injection can be facilitated by an intraventricular catheter, for example, attached to a reservoir, such as an Ommaya reservoir.

In one embodiment, the compound or a pharmaceutically acceptable salt of the compound is formulated in accordance with routine procedures as a composition adapted for oral administration to humans. Compositions for oral delivery can be in the form of tablets, lozenges, buccal forms, troches, aqueous or oily suspensions or solutions, granules, powders, emulsions, capsules, syrups, elixirs, oral liquids, suspensions or solutions, for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Oral formulations may utilize standard delay or time release formulations to alter the absorption of the compound or pharmaceutically acceptable salt of the compound. The oral formulation may also consist of administering the compound or pharmaceutically acceptable salt of the compound in water or fruit juice, containing appropriate solubilizers or emulisifiers as needed.

The compound or a pharmaceutically acceptable salt of the compound can be administered by controlled-release or sustained-release means or by delivery devices that are known to those of ordinary skill in the art (see, e.g., Goodson, in Medical Applications of Controlled Release, vol. 2, pp. 115-138 (1984)).

Liquid pharmaceutical compositions, which are sterile solutions or suspensions, can be utilized by, for example, intramuscular, intraperitoneal or subcutaneous injection. Sterile solutions can also be administered intravenously. Compositions for oral administration may be in either liquid or solid form.

Pulmonary administration can also be employed, e.g., by use of an inhaler or nebulizer, and formulation with an aerosolizing agent, or via perfusion in a fluorocarbon or synthetic pulmonary surfactant. In some embodiments, the compounds or pharmaceutically acceptable salts of the compounds are administered directly to the airways in the form of an aerosol. For administration by intranasal or intrabronchial inhalation, the compounds or pharmaceutically acceptable salts of the compounds may be formulated into an aqueous or partially aqueous solution

The pharmaceutical forms suitable for injectable use include sterile aqueous solutions or dispersions and sterile powders for the extemporaneous preparation of sterile injectable solutions or dispersions. In some embodiments, the form is sterile and is fluid to the extent that easy syringability exists. Such dosage forms are generally stable under the conditions of manufacture and storage and are preserved against the contaminating action of microorganisms such as bacteria and fungi.

The compounds or pharmaceutically acceptable salts of the compounds described herein can be administered transdermally through the use of a transdermal patch. Transdermal administrations include administrations across the surface of the body and the inner linings of bodily passages including epithelial and mucosal tissues. Such administrations may be carried out using the compounds described herein, or pharmaceutically acceptable salts thereof, in lotions, creams, foams, patches, suspensions, solutions, and suppositories (rectal and vaginal).

Transdermal administration may be accomplished through the use of a transdermal patch containing the compound or pharmaceutically acceptable salt of the compound and a carrier that is inert to the compound, is non-toxic to the skin, and allows delivery of the compound for systemic absorption into the blood stream via the skin. The carrier may take any number of forms such as creams and ointments, pastes, gels and occlusive devices.

The compounds or pharmaceutically acceptable salts of the compounds described herein may be administered rectally or vaginally in the form of a conventional suppository. Suppository formulations may be made from traditional binders and excipients, including cocoa butter and triglycerides, with or without the addition of waxes to alter the suppository's melting point, and glycerin.

It is understood that the dosage, regimen and mode of administration of these compounds will vary according to the malady and the individual being treated and will be subject to the judgment of the medical practitioner involved. In some embodiments, administration of one or more of the compounds or pharmaceutically acceptable salts of the compounds described herein begins at a low dose and is increased until the desired effects are achieved.

When administered for the treatment or inhibition of a particular disease state or disorder, it is understood that the effective dosage may vary depending upon the particular compound or pharmaceutically acceptable salt of the compound utilized, the mode of administration, the condition, and severity thereof, of the condition being treated, as well as the various physical factors related to the individual being treated. In therapeutic application, the compounds and pharmaceutically acceptable salts of the compounds described herein are provided to a patient already suffering from a disease in an amount sufficient to cure or at least partially ameliorate the symptoms of the disease and its complications. An amount adequate to accomplish this is defined as a “therapeutically effective amount”. The dosage to be used in the treatment of a specific case must be subjectively determined by the attending physician. The variables involved include the specific condition and the size, age and response pattern of the patient.

In one embodiment a controlled- or sustained-release composition includes a minimal amount of the compound or a pharmaceutically acceptable salt of the compound to treat or prevent a central nervous system disorder in a minimal amount of time. Advantages of controlled- or sustained-release compositions include extended activity of the drug, reduced dosage frequency, and increased compliance by the animal being treated. In addition, controlled or sustained release compositions can favorably affect the time of onset of action or other characteristics, such as blood levels of the compound or a pharmaceutically acceptable salt of the compound, and can thus reduce the occurrence of adverse side effects.

Controlled- or sustained-release compositions can initially release an amount of the compound or a pharmaceutically acceptable salt of the compound that promptly produces the desired therapeutic or prophylactic effect, and gradually and continually release other amounts of the compound or a pharmaceutically acceptable salt of the compound to maintain this level of therapeutic or prophylactic effect over an extended period of time. To maintain a constant level of the compound or a pharmaceutically acceptable salt of the compound in the body, the compound or a pharmaceutically acceptable salt of the compound can be released from the dosage form at a rate that will replace the amount of the compound or a pharmaceutically acceptable salt of the compound being metabolized and excreted from the body. Controlled or sustained release of the compound or pharmaceutically acceptable salt of the compound can be stimulated by various conditions, including but not limited to, changes in pH, changes in temperature, concentration or availability of enzymes, concentration or availability of water, or other physiological conditions or compounds.

The amount of the compound or a pharmaceutically acceptable salt of the compound delivered is an amount that is effective for treating or preventing a central nervous system disorder. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. The effective dosage amounts described herein refer to total amounts administered; that is, if more than one compound or a pharmaceutically acceptable salt of the compound is administered, the effective dosage amounts correspond to the total amount administered.

The amount of the compound or a pharmaceutically acceptable salt of the compound that is effective for treating or preventing a central nervous system disorder will typically range from about 0.001 mg/kg to about 600 mg/kg of body weight per day, in one embodiment, from about 1 mg/kg to about 600 mg/kg body weight per day, in one embodiment, from about 1 mg/kg to about 250 mg/kg body weight per day, in another embodiment, from about 10 mg/kg to about 400 mg/kg body weight per day, in another embodiment, from about 10 mg/kg to about 200 mg/kg of body weight per day, in another embodiment, from about 10 mg/kg to about 100 mg/kg of body weight per day, in one embodiment, from about 10 mg/kg to about 25 mg/kg body weight per day, in another embodiment, from about 1 mg/kg to about 10 mg/kg body weight per day, in another embodiment, from about 0.001 mg/kg to about 100 mg/kg of body weight per day, in another embodiment, from about 0.001 mg/kg to about 10 mg/kg of body weight per day, and in another embodiment, from about 0.001 mg/kg to about 1 mg/kg of body weight per day.

In one embodiment, the pharmaceutical composition is in unit dosage form, e.g., as a tablet, capsule, powder, solution, suspension, emulsion, granule, or suppository. In such form, the composition is sub-divided in unit dose containing appropriate quantities of the compound or pharmaceutically acceptable salt of the compound; the unit dosage form can be packaged compositions, for example, packeted powders, vials, ampoules, prefilled syringes or sachets containing liquids. The unit dosage form can be, for example, a capsule or tablet itself, or it can be the appropriate number of any such compositions in package form. Such unit dosage form may contain from about 0.01 mg/kg to about 250 mg/kg, in one embodiment from about 1 mg/kg to about 250 mg/kg, in another embodiment from about 10 mg/kg to about 25 mg/kg, and may be given in a single dose or in two or more divided doses. Variations in the dosage will necessarily occur depending upon the species, weight and condition of the patient being treated and the patient's individual response to the medicament.

In one embodiment, the unit dosage form is about 0.01 to about 1000 mg. In another embodiment, the unit dosage form is about 0.01 to about 500 mg; in another embodiment, the unit dosage form is about 0.01 to about 250 mg; in another embodiment, the unit dosage form is about 0.01 to about 100 mg; in another embodiment, the unit dosage form is about 0.01 to about 50 mg; in another embodiment, the unit dosage form is about 0.01 to about 25 mg; in another embodiment, the unit dosage form is about 0.01 to about 10 mg; in another embodiment, the unit dosage form is about 0.01 to about 5 mg; and in another embodiment, the unit dosage form is about 0.01 to about 10 mg;

The compound or a pharmaceutically acceptable salt of the compound can be assayed in vitro or in vivo for the desired therapeutic or prophylactic activity prior to use in humans. Animal model systems can be used to demonstrate safety and efficacy.

The present methods for treating or preventing a central nervous system disorder can further include administering another therapeutic agent to the animal being administered the compound or a pharmaceutically acceptable salt of the compound. In one embodiment the other therapeutic agent is administered in an effective amount.

Effective amounts of the other therapeutic agents are well known to those skilled in the art. However, it is well within the skilled artisan's purview to determine the other therapeutic agent's optimal effective amount range. The compound or a pharmaceutically acceptable salt of the compound and the other therapeutic agent can act additively or, in one embodiment, synergistically. In one embodiment, where another therapeutic agent is administered to an animal, the effective amount of the compound or a pharmaceutically acceptable salt of the compound is less than its effective amount would be where the other therapeutic agent is not administered. In this case, without being bound by theory, it is believed that the compound or a pharmaceutically acceptable salt of the compound and the other therapeutic agent act synergistically. In some cases, the patient in need of treatment is being treated with one or more other therapeutic agents. In some cases, the patient in need of treatment is being treated with at least two other therapeutic agents. In one embodiment, the other therapeutic agent is selected from the group consisting of one or more anti-depressant agents (e.g., SSRIs, monoamine oxidase inhibitors, norepinephrine reuptake inhibitors, and serotonin and noradrenaline reuptake inhibitors), anti-anxiety agents (e.g., benzodiazepines, serotonin 1A (5-HT_{1A}) agonists or antagonists (such as 5-HT_{1A} partial agonists), or corticotrophin releasing factor), anti-psychotic agents (e.g., phethiazine, piperainze phenothiazines, butyrophenones, substituted benzamides, thioxanthine, haloperidol, olanzapine, clozapine, risperidone, pimozide, aripiprazol, or ziprasidone), or cognitive enhancers (e.g., acetylcholinesterase or cholinesterase inhibitors, cholinergic receptor agonists, or serotonin receptor antagonists, drugs that modulate the level of soluble Aβ amyloid fibril formation or amyloid plaque burden, or drugs that protect neuronal activity).

In one embodiment, the compound or a pharmaceutically acceptable salt of the compound is administered concurrently with another therapeutic agent.

In one embodiment, a composition including an effective amount of the compound or a pharmaceutically acceptable salt of the compound and an effective amount of another therapeutic agent within the same composition can be administered.

In another embodiment, a composition including an effective amount of the compound or a pharmaceutically acceptable salt of the compound and a separate composition including an effective amount of another therapeutic agent can be concurrently administered. In another embodiment, an effective amount of the compound or a pharmaceutically acceptable salt of the compound is administered prior to or subsequent to administration of an effective amount of another therapeutic agent. In this embodiment, the compound or a pharmaceutically acceptable salt of the compound is administered while the other therapeutic agent exerts its therapeutic effect, or the other therapeutic agent is administered while the compound or a pharmaceutically acceptable salt of the compound exerts its preventative or therapeutic effect for treating or preventing a central nervous system disorder.

Thus, in one embodiment, a composition including an effective amount of a compound described herein or a pharmaceutically acceptable salt of a compound described herein and a pharmaceutically acceptable carrier is provided. In another embodiment, the composition further includes a second therapeutic agent. In some embodiments, the second therapeutic agent includes one or more other antidepressants, anti-anxiety agents, anti-psychotic agents or cognitive enhancers. In another embodiment, the pharmaceutically acceptable carrier is suitable for oral administration and the composition includes an oral dosage form.

In one embodiment, the compounds or pharmaceutically acceptable salts of the compounds described herein are useful as modulators of the activity of a 5-HT_{1A} receptor. As 5-HT_{1A} agonists, partial agonists, or antagonists, the compounds described herein are useful for the treatment and/or prevention of several diseases and disorders related to the 5-HT_{1A} receptor. For example, in one embodiment, the compounds or pharmaceutically acceptable salts of the compounds described herein bind to a 5-HT_{1A} receptor. In one embodiment, the compounds or pharmaceutically acceptable salts of the compounds described herein are useful as 5-HT_{1A} receptor antagonists. Compounds that modulate the activity of 5-HT_{1A} receptors, such as for example by binding to or antagonizing the receptor, can be readily identified by those skilled in the art using numerous art-recognized methods, including standard pharmacological test procedures such as those described herein. Accordingly, in some embodiments, the compounds and pharmaceutically acceptable salts of the compounds described herein are useful for treating a mammal with a central nervous system disorder that is mediated through the 5-HT_{1A} pathway. Central nervous system disorders include, without limitation, anxiety-related disorders, cognition-related disorders, depression and depression-related disorders, and schizophrenia and other psychotic disorders. Thus, in one embodiment, the compounds and pharmaceutically acceptable salts of the compounds described herein that act as 5-HT_{1A} receptor modulators are useful for treating a mammal with a cognition-related disorder, an anxiety-related disorder, depression or schizophrenia.

Exemplary cognition-related disorders (e.g., cognitive dysfunction) include, without limitation, mild cognitive impairment (MCI), dementia, delirium, amnestic disorder, Alzheimer's disease, Parkinson's disease, Huntington's disease, memory disorders including memory deficits associated with depression, senile dementia, dementia of Alzheimer's disease, cognitive deficits or cognitive dysfunction associated with neurological conditions including, for example, Parkinson's disease (PD), Huntington's disease (HD), Alzheimer's disease, depression and schizophrenia (and other psychotic disorders such as paranoia and mano-depressive illness); cognitive dysfunction in schizophrenia, disorders of attention and learning such as attention deficit disorders (e.g., attention deficit hyperactivity disorder (ADHD)) and dyslexia, cognitive dysfunction associated with developmental disorders such as Down's syndrome and Fragile X syndrome, loss of executive function, loss of learned information, vascular dementia, schizophrenia, cognitive decline, neurodegenerative disorder, and other dementias, for example, due to HIV disease, head trauma, Parkinson's disease, Huntington's disease, Pick's disease, Creutzfeldt-Jakob disease, or due to multiple etiologies. Cognition-related disorders also include, without limitation, cognitive dysfunction associated with MCI and dementias such as Lewy Body, vascular, and post stroke dementias. Cognitive dysfunction associated with surgical procedures, traumatic brain injury or stroke may also be treated in accordance with the embodiments described herein.

Exemplary anxiety-related disorders include, without limitation, generalized anxiety disorder, attention deficit disorder, attention deficit hyperactivity disorder, obsessive compulsive disorder, substance addiction, withdrawal from drug, alcohol or nicotine addiction, panic disorder, panic attacks, post traumatic stress disorder, premenstrual dysphoric disorder, social anxiety disorder, eating disorders such as anorexia nervosa and bulimia nervosa, vasomotor flushing, and phobias, including social phobia, agoraphobia, and specific phobias. Substance addition includes, without limitation, drug, alcohol or nicotine addiction.

Compounds of Formulas I, II and III have been found to act as serotonin reuptake inhibitors and to have an affinity for the 5-HT_{1A} reuptake transporter. They are, therefore, useful in the treatment of diseases affected by disorders of the serotonin affected neurological systems. Accordingly, in one embodiment, the compounds or pharmaceutically acceptable salts of the compounds described herein are useful as modulators of serotonin reuptake. For example, in some embodiments, the compounds or pharmaceutically acceptable salts of the compounds described herein can block the reuptake of the brain neurotransmitter serotonin. Accordingly, the compounds or pharmaceutically acceptable salts of the compounds described herein are useful for the treatment or prevention of conditions commonly treated by the administration of serotonin selective reuptake inhibitor (SSRI) antidepressants, such as depression (including but not limited to major depressive disorder, childhood depression, dysthymia, and depression associated with stroke), anxiety, panic disorder, post-traumatic stress disorder, premenstrual dysphoric disorder (e.g., pre-menstrual syndrome), attention deficit disorder (with and without hyperactivity), obsessive compulsive disorders (including but not limited to trichotillomania), obsessive compulsive spectrum disorders (including but not limited to autism), social anxiety disorder, generalized anxiety disorder, obesity, eating disorders such as anorexia nervosa, bulimia nervosa, vasomotor flushing, cocaine and alcohol addiction, sexual dysfunction (including but not limited to premature ejaculation), incontinence (including, but not limited to fecal incontinence, urge incontinence, overflow incontinence, passive incontinence, reflex incontinence, stress urinary incontinence urinary exertional incontinence and urinary incontinence), and pain (including, but not limited to migraine, chronic back pain, phantom limb pain, neuropathic pain such as diabetic neuropathy, and post herpetic neuropathy) and related illnesses.

The compounds or pharmaceutically acceptable salts of the compounds described herein are also useful for the treatment or prevention of conditions mediated through the 5-HT_{1A} receptors (e.g., those commonly treated by the administration of 5-HT_{1A} antagonists), such as depression, such as single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder, pediatric depression, child abuse induced depression and postpartum depression; bipolar disorders or manic depression, for example, bipolar I disorder, bipolar II disorder and cyclothymic disorder; conduct disorder; disruptive behavior disorder; disorders of attention and learning such as attention deficit hyperactivity disorder (ADHD) and dyslexia; behavioral disturbances associated with mental retardation, autistic disorder, pervasive development disorder and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia, substance-induced psychotic disorder, shared psychotic disorder, and psychotic disorder due to a general medical condition; delirium, dementia, and amnestic and other cognitive or neurodegenerative disorders, such as Parkinson's disease (PD), Huntington's disease (HD), Alzheimer's disease, senile dementia, dementia of the Alzheimer's type, mild cognitive impairment (MCI), memory disorders, loss of executive function, vascular dementia, and other dementias, for example, due to HIV disease, head trauma, Parkinson's disease, Huntington's disease, Pick's disease, Creutzfeldt Jakob disease, or due to multiple etiologies; cognitive deficits associated with neurological conditions including, for example, Parkinson's disease (PD), Huntington's disease (HD), Alzheimer's disease; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremor; chemical dependencies and addictions (e.g., dependencies on, or addictions to, alcohol, heroin, cocaine, benzodiazepines, nicotine, or phenobarbitol); behavioral addictions such as an addiction to gambling; and ocular disorders such as glaucoma and ischemic retinopathy; sexual dysfunction associated with drug treatment (e.g., sexual dysfunction associated with SSRI's).

The compounds described herein are useful use as active therapeutic substances. Compounds described herein are of particular use in the treatment of diseases affected by disorders of serotonin.

In one embodiment, methods for treating depression (including, but not limited to major depressive disorder, childhood depression and dysthymia), anxiety, panic disorder, post-traumatic stress disorder, premenstrual dysphoric disorder (also known as premenstrual syndrome), attention deficit disorder (with or without hyperactivity), obsessive compulsive disorder, social anxiety disorder, generalized anxiety disorder, obesity, eating disorders such as anorexia nervosa and bulimia nervosa, vasomotor flushing, cocaine and alcohol addiction, sexual dysfunction, and related illnesses in mammals including man are provided. The methods include administering to the afflicted mammal an effective amount of a compound described herein or a pharmaceutical composition containing one or more of the compounds described herein.

In some embodiments, the compounds and pharmaceutically acceptable salts of the compounds described herein have dual-acting mechanisms. That is, the compounds and pharmaceutically acceptable salts of the compounds have an ability to modulate serotonin reuptake, as well as an ability to modulate 5-HT_{1A} receptors (e.g., through binding or antagonism).

In one embodiment, a method for modulating the activity of a 5-HT_{1A} receptor is provided. The method includes contacting the receptor with one or more compounds or pharmaceutically acceptable salts of the compounds described herein. In one embodiment, a method of binding a 5-HT_{1A} receptor in a patient is provided. The method includes administering to the patient an effective amount of one or more compounds or pharmaceutically acceptable salts of the compounds described herein. In one embodiment, a method of antagonizing a 5-HT_{1A} receptor is provided. The method includes administering an effective amount of one or more compounds or pharmaceutically acceptable salts of the compounds described herein. In some embodiments, the method includes administration to a patient suffering from a 5-HT_{1A}-related disorder. In one embodiment, a method of modulating serotonin reuptake in a patient is provided. The method includes administering an effective amount of one or more compounds or pharmaceutically acceptable salts of the compounds described herein.

In one embodiment, a method for treating depression, including administering to a mammal in need thereof a compound or a pharmaceutically acceptable salt of a compound or pharmaceutically acceptable salt of a compound described herein in an amount effective to treat depression is provided. In one embodiment, the method for treating depression includes administering a second therapeutic agent. In some embodiments, the second therapeutic agent is an anti-depressant agent, an anti-anxiety agent, an anti-psychotic agent, or a cognitive enhancer.

In some embodiments, pharmaceutical compositions or medicaments are provided. The pharmaceutical compositions or medicaments include one or more compounds or pharmaceutically acceptable salts of the compounds described herein. In some embodiments, the pharmaceutical compositions also include one or more pharmaceutically acceptable carriers. In some embodiments, the pharmaceutical composition is useful for modulating the activity of a 5-HT_{1A} receptor (e.g., by binding or antagonizing the receptor). In some embodiments, the pharmaceutical composition is useful for modulating serotonin reuptake in a patient. In some embodiments, the pharmaceutical composition is useful for treating a central nervous system disorder. In one embodiment, a pharmaceutical composition for treating depression is provided. The composition includes a compound or a pharmaceutically acceptable salt of a compound described herein.

In some embodiments, the compounds or pharmaceutically acceptable salts of the compounds described herein demonstrate fewer undesired side effects than observed for other central nervous system treatments. For example, a side effect associated with some antidepressant therapy (such as SSRIs) is sexual dysfunction. In some embodiments, administration of the compounds or pharmaceutically acceptable salts of the compounds described herein demonstrate a lower incidence or degree of sexual dysfunction than that associated with SSRI antidepressants, tricyclic antidepressants, aminoketone class compounds, monoamine oxidase inhibitors (MAOI), serotonin and norepinepherine reuptake inhibitors (SNRI), norepinephrine reuptake inhibitor (NRI), partial 5-HT_{1A} agonists, 5-HT_{2A} receptor antagonists, or antipsychotic drug (typical and atypical). Accordingly, in one embodiment, the compounds or pharmaceutically acceptable salts of the compounds described herein are useful for treating sexual dysfunction, e.g., sexual dysfunction associated with drug treatment such as drug treatment with an antidepressant, an antipsychotic, or an anticonvulsant. In some embodiments, the sexual dysfunction includes a deficiency in penile erection.

The compounds and pharmaceutically acceptable salts of the compounds described herein are also useful in the manufacture of medicaments for treating a central nervous system disorder in a mammal. Similarly, the compounds and pharmaceutically acceptable salts of the compounds described herein are also useful in the manufacture of medicaments for treating a cognition-related disorder, an anxiety-related disorder, depression, or schizophrenia in a mammal. Also, the compounds and pharmaceutically acceptable salts of the compounds of described herein are useful in the manufacture of medicaments for modulating the activity of a 5-HT_{1A} receptor in a mammal. In some embodiments, the compounds and pharmaceutically acceptable salts of the compounds of described herein are also useful in the manufacture of medicaments for modulating serotonin reuptake in a mammal.

The following examples illustrate the production of representative compounds described herein.

### EXAMPLES

### Example 1

### (3R)-3-[3-(5-fluoro-1H-indol-3-yl)propyl]amino-8-methoxychromane-5-carboxamide

(3R)-3-[3-(5-fluoro-1H-indol-3-yl)propyl]amino-8-fluorochromane-5-carboxamide (4.00 g, 10.39 mmol) was dissolved in MeOH (5.00 mL) and then treated with NaOMe (25% wt in MeOH; 20.00 mL) this solution was heated at reflux overnight, afterwhich LC/MS indicated reaction was complete. The reaction mixture was then diluted with H₂O and treated with NaOH until pH˜11. The reaction mixture was then extracted with EtOAc, and the organic layers were combined, washed with brine, dried over Na₂SO₄, and concentrated under reduced pressure to afford the title compound as a white crystalline solid (3.5 g, 85%); MS (ES) m/z=398.2 [M+H]+, 420.2 [M+Na]+, 795.4 [2M+H]+.

### Example 2

### (3R)-3-{cyclobutyl[3-(5-fluoro-1H-indol-3-yl)propyl]amino}-8-methoxychromane-5-carboxamide

(3R)-3-{[3-(5-fluoro-1H-indol-3-yl)propyl]amino}-8-methoxychromane-5-carboxamide (2.50 g, 6.29 mmol) was dissolved in MeOH (70.00 mL) and treated with cyclobutanone (1.65 mL, 22.04 mmol), AcOH (0.83 mL, 13.21 mmol) and NaCNBH₃ (0.83 g, 13.21 mmol). This mixture was stirred under an inert atmosphere for 3 days, after which LC/MS indicated reaction was complete. The reaction mixture was quenched with NaOH (1N) until pH˜10, and then extracted with CH₂Cl₂. The organic layer was then washed with brine, dried over Na₂SO₄, and concentrated under reduced pressure. The crude material was subjected to silica gel chromatography to afford title compound as a colorless oil (1.90 g, 68%); MS (ES) m/z=452.2 [M+H]+.

### Example 3

### (3R)-3-{cyclobutyl[3-(5-fluoro-1H-indol-3-yl)propyl]amino}-8-ethoxychromane-5-carboxamide

Prepared as described for examples 1 and 2 by using ethanol and sodium ethoxide instead of methanol and sodium methoxide in example 1. MS (ES) m/z=466.2 [M+H]+.

### Example 4

### (3R)-3-{cyclobutyl[3-(5-fluoro-1H-indol-3-yl)propyl]amino}-8-propoxychromane-5-carboxamide

Prepared as described for examples 1 and 2 by using propanol and sodium propoxide instead of methanol and sodium methoxide in example 1. MS (ES) m/z=480.3 [M+H]+.

### Example 5

### (3R)-3-{cyclobutyl[3-(5-fluoro-1H-indol-3-yl)propyl]amino}-8-isopropoxychromane-5-carboxamide

Prepared as described for examples 1 and 2 by using isopropanol and sodium isopropoxide instead of methanol and sodium methoxide in example 1. MS (ES) m/z=480.3 [M+H]+.

### Example 6

### (3R)-3-{cyclobutyl[3-(5-fluoro-1H-indol-3-yl)propyl]amino}-8-hydroxychromane-5-carboxamide

(3R)-3-{cyclobutyl[3-(5-fluoro-1H-indol-3-yl)propyl]amino}-8-methoxychromane-5-carboxamide (0.36 g, 0.79 mmol) was dissolved in CH₂Cl₂ (8.00 mL) and subsequently treated with BBr₃ (1.0M in CH₂Cl₂; 9.00 mL). After 4 hours, the reaction appeared complete by TLC and LC/MS analysis. The reaction mixture was cooled to 0° C., and carefully quenched with aqueous NaHCO₃. Once quenched, the mixture was extracted with CH₂Cl₂; the resulting organic layer was then washed with brine, dried over anhydrous Na₂SO₄, and concentrated under reduced pressure. The crude material was subjected to silica gel chromatography to afford the title compound as a white solid (0.24 g, 70%); MS (ES) m/z=438.2 [M+H]+, 460.2 [M+Na]+.

### Example 7

### (3R)-3-{3,3,3-trifluoropropyl[3-(5-fluoro-1H-indol-3-yl)propyl]amino}-8-methoxychromane-5-carboxamide

Prepared as described for example 2 by using 3,3,3-trifluoropropaldehyde instead of cyclobutanone in example 1. MS (ES) m/z=494.2 [M+H]+.

### Example 8

### (3R)-3-{cyclopropylmethyl[3-(5-fluoro-1H-indol-3-yl)propyl]amino}-8-methoxychromane-5-carboxamide

Prepared as described for example 2 by using cyclopropylformaldehyde instead of cyclobutanone in example 1. MS (ES) m/z=452.3 [M+H]+.

### Example 9

### (3R)-3-{2-isopropyl[3-(5-fluoro-1H-indol-3-yl)propyl]amino}-8-methoxychromane-5-carboxamide

Prepared as described for example 2 by using acetone instead of cyclobutanone in example 1. MS (ES) m/z=440.3 [M+H]+.

### Example 10

### (3R)-3-{propyl[3-(5-fluoro-1H-indol-3-yl)propyl]amino}-8-methoxychromane-5-carboxamide

Prepared as described for example 2 by using propaldehyde instead of cyclobutanone in example 1. MS (ES) m/z=440.2 [M+H]+.

### Example 11

### (3R)-3-{butyl[3-(5-fluoro-1H-indol-3-yl)propyl]amino}-8-methoxychromane-5-carboxamide

Prepared as described for example 2 by using n-butylaldehyde instead of cyclobutanone in example 1. MS (ES) m/z=454.3 [M+H]+.

### Example 12

### (3R)-3-{4,4,4-trifluorobutyl[3-(5-fluoro-1H-indol-3-yl)propyl]amino}-8-methoxychromane-5-carboxamide

Prepared as described for example 2 by using 4,4,4-trifluorobutylaldehyde instead of cyclobutanone in example 1. MS (ES) m/z=508.2 [M+H]+.

### Example 13

### (3R)-3-{2-methylpropyl[3-(5-fluoro-1H-indol-3-yl)propyl]amino}-8-methoxychromane-5-carboxamide

Prepared as described for example 2 by using 2-methylpropaldehyde instead of cyclobutanone in example 1. MS (ES) m/z=454.2 [M+H]+.

### Example 14

### (3R)-3-{ethyl[3-(5-fluoro-1H-indol-3-yl)propyl]amino}-8-methoxychromane-5-carboxamide

Prepared as described for example 2 by using acetaldehyde instead of cyclobutanone in example 1. MS (ES) m/z=426.2 [M+H]+.

### Example 15

### (3R)-3-{methyl[3-(5-fluoro-1H-indol-3-yl)propyl]amino}-8-methoxychromane-5-carboxamide

Prepared as described for example 2 by using formaldehyde instead of cyclobutanone in example 1. MS (ES) m/z=412 [M+H]+.

### Example 16

### (3R)-3-{cyclobutyl[3-(5-fluoro-1H-indol-3-yl)propyl]amino}-8-methoxychromane-5-methylcarboxamide

(3R)-3-{[3-(5-fluoro-1H-indol-3-yl)propyl]amino}-8-methoxychromane-5-carboxamide (500 mg) was dissolved in methanol (50 mL) and treated with concentrated sulfuric acid (1 mL). The reaction mixture was stirred at reflux for 20 hours. The cooled reaction mixture was concentrated under reduced pressure and extracted with methylene chloride (3×) from saturated sodium bicarbonate solution, dried (Na₂SO₄) and concentrated in vacuo to afford the ester product. This intermediate was dissolved in THF (50 mL), treated with a solution of LiOH in water (1.0 M, 2.0 mL) and stirred at room temperature for 20 hours. The resulting mixture was concentrated in vacuo to afford the lithium salt of (3R)-3-{[3-(5-fluoro-1H-indol-3-yl)propyl]amino}-8-methoxychromane-5-carboxylic acid as white solid (400 mg). MS (ES) m/z=399.2 [M+H]+.

100 mg of this carboxylic acid was dissolved in methanol (2.5 mL), treated with cyclobutanone (0.11 mL), AcOH (0.1 mL) and NaBH₃CN (100 mg) sequentially, stirred at room temperature for 2 days. Reaction mixture was quenched with water, extracted with CH₂Cl₂ (2×) from saturated sodium bicarbonate, dried (Na₂SO₄) and concentrated. Purification by HPLC afforded (3R)-3-{cyclobutyl[3-(5-fluoro-1H-indol-3-yl)propyl]amino}-8-methoxychromane-5-carboxamide as white solid. This product was dissolved in THF (3 mL), treated with methylamine in THF (2.0 M, 0.3 mL), EDC (30 mg), HOBt (57 mg) sequentially and stirred at room temperature for 2 days. Reaction mixture was diluted with methanol and purified by HPLC to afford (3R)-3-{[3-(5-fluoro-1H-indol-3-yl)propyl]amino}-8-methoxychromane-5-methylcarboxamide as white solid (50 mg). MS (ES) m/z=466.3 [M+H]+.

### Example 17

### (3R)-3-{3,3,3-trifluoropropyl[3-(5-fluoro-1H-indol-3-yl)propyl]amino}-8-methoxychromane-5-methylcarboxamide

Prepared as described for example 16 by using 3,3,3-trifluoropropaldehyde instead of cyclobutanone in example 16. MS (ES) m/z=508.3 [M+H]+.

### Example 18

### (3R)-3-{cyclobutyl[3-(5,7-difluoro-1H-indol-3-yl)propyl]amino}-8-methoxy-chromane-5-carboxamide

Prepared as described for examples 1 and 2 by using (3R)-3-{[3-(5,7-difluoro-1H-indol-3-yl)propyl]amino}-8-fluorochromane-5-carboxamide as the starting material instead of (3R)-3-{[3-(5-fluoro-1H-indol-3-yl)propyl]amino}-8-fluorochromane-5-carboxamide as in example 1. MS (ES) m/z=470.2 [M+H]+.

### Example 19

### (3R)-3-{cyclobutyl[3-(5-fluoro-1H-indolin-3-yl)propyl]amino}-8-methoxy-chromane-5-carboxamide

(3R)-3-{cyclobutyl[3-(5-fluoro-1H-indol-3-yl)propyl]amino}-8-methoxychromane-5-carboxamide (120 mg) was dissolved in TFA (2.0 mL), treated with NaBH₃CN (100 mg) portion-wise, stirred under nitrogen atmosphere at room temperature for 2 hours. The reaction mixture was then carefully quenched with a solution of NaOH in water (50%), extracted with CH₂Cl₂ (3×) from saturated NaHCO₃, dried (Na₂SO₄) and concentrated under reduced pressure. Chromatography with MeOH—CH₂Cl₂ (0-20% gradient) afforded the title compound as white solid (50 mg). MS (ES) m/z=454.3 [M+H]+.

### Example 20

### (3R)-3-{cyclobutyl[3-(5-fluoro-1H-indol-3-yl)propyl]amino}-8-methylchromane-5-carboxamide

(3R)-3-{cyclobutyl[3-(5-fluoro-1H-indol-3-yl)propyl]amino}-8-hydroxychromane-5-carboxamide (0.30 g, 0.69 mmol) was dissolved in pyridine (3.00 mL) and cooled to 0° C., afterwhich, triflic anhydride (0.14 mL, 0.82 mmol) was added dropwise. This solution was allowed to slowly warm to room temperature and was then stirred for an additional hour. LC/MS indicated that reaction was complete, so reaction mixture was diluted with ether and was then washed with a solution of saturated NaHCO₃, followed by H₂O, and finally brine. This resulting organic layer was then dried over Na₂SO₄, and concentrated under reduced pressure. Crude material was subjected to silica gel chromatography to afford the triflate (0.20 g, 50%) needed for the next step. This triflate (0.075 g, 0.132 mmol) was then dissolved in DMF (3.00 mL), and the flask was covered in aluminum foil to keep reaction free of light. This mixture was then treated with SnMe₄ (0.18 mL, 1.32 mmol) and tetrakis (triphenylphosphine) palladium(0) (0.015 g, 0.013 mmol). This reaction mixture was heated at 140° C. under an inert atmosphere. After stirring overnight, LC/MS indicated the product had been formed. The reaction mixture was cooled to room temperature, diluted with a saturated solution of NaHCO₃, and extracted with CH₂Cl₂. The organic layer was washed with brine, dried over Na₂SO₄, and concentrated under reduced pressure. The resulting crude material was purified by HPLC to afford the title compound; MS (APPI) m/z=436 [M+H]+.

### Example 21

### (3R)-3-{cyclobutyl[3-(5-fluoro-1H-indol-3-yl)propyl]amino}-8-ethylchromane-5-carboxamide

(3R)-3-{cyclobutyl[3-(5-fluoro-1H-indol-3-yl)propyl]amino}-8-hydroxychromane-5-carboxamide (0.30 g, 0.69 mmol) was dissolved in pyridine (3.00 mL) and cooled to 0° C., after which triflic anhydride (0.14 mL, 0.82 mmol) was added drop wise. This solution was allowed to slowly warm to room temperature and was then stirred for an additional hour. LC/MS indicated that reaction was complete, so reaction mixture was diluted with ether and was then washed with a solution of saturated NaHCO₃, followed by H₂O, and finally brine. The resulting organic layer was then dried over Na₂SO₄, and concentrated under reduced pressure. Crude material was subjected to silica gel chromatography to afford the triflate (0.20 g, 50%) needed for the next step. This triflate (0.086 g, 0.15 mmol) was then dissolved in DMF (1.50 mL), and the flask was covered in aluminum foil to keep reaction free of light. This mixture was then treated with SnMe₄ (0.29 mL, 1.46 mmol) and tetrakis (triphenylphosphine) palladium(0) (0.017 g, 0.015 mmol). This reaction mixture was heated at 140° C. under an inert atmosphere. After stirring overnight, LC/MS indicated product had been formed. The reaction mixture was cooled to room temperature, diluted with a saturated solution of NaHCO₃, and extracted with CH₂Cl₂. The organic layer was washed with brine, dried over Na₂SO₄, and concentrated under reduced pressure. The resulting crude material was purified by HPLC to afford the title compound; MS (ES) m/z=450.2 [M+H]+, 472.2 [M+Na]+.

### Example 22

### (3R)-3-{cyclobutyl[3-(5-fluoro-1H-indol-3-yl)propyl]amino}-8-(methylthio)chromane-5-carboxamide

(3R)-8-fluoro-3-{[3-(5-fluoro-1H-indol-3-yl)propyl]amino}chromane-5-carboxamide (0.50 g, 1.30 mmol) was dissolved in DMSO (5.00 mL) and treated with NaSMe (0.182 g, 2.60 mmol). After stirring overnight at 100° C., reaction was cooled to room temperature, and queched with NaHCO₃. This mixture was extracted with CH₂Cl₂; the organic layer was washed with H₂O followed by brine, then dried over Na₂SO₄, and concentrated to give an off-white solid. Crude product was used directly in the next step without further purification. The crude product (0.50 g, 1.21 mmol) was dissolved in MeOH (15.00 mL), treated with cyclobutanone (0.32 mL, 4.23 mmol), AcOH (0.16 mL, 2.54 mmol), and NaCNBH₃ (0.16 g, 2.54 mmol). The reaction mixture was allowed to stir at room temperature overnight, after which TLC and LC/MS indicated reaction was complete. The mixture was treated with NaOH (30%) until pH˜10, and was then concentrated under reduced pressure to eliminate of most of the MeOH. The resulting material was then diluted with CH₂Cl₂, washed with H₂O followed by brine, dried over Na₂SO₄, and concentrated under reduced pressure. This light yellow oil was purified by HPLC to afford the title compound; MS (ES) m/z=468.2 [M+H]+, 490.2 [M+Na]+.

### Example 23

### (3R)-3-{cyclobutyl[3-(5-fluoro-1H-indol-3-yl)propyl]amino}-8-(ethylthio)chromane-5-carboxamide

To a suspension of EtSH (2.00 mL, 27.00 mmol) and NaH (0.80 g, 33.00 mmol) was added drop wise a solution of (3R)-8-fluoro-3-{[3-(5-fluoro-1H-indol-3-yl)propyl]amino}chromane-5-carboxamide (0.14 g, 0.36 mmol) dissolved in DMF (0.5 mL). This reaction was allowed to stir under an inert atmosphere for 3 days, and was then quenched with NaHCO₃. The reaction mixture was extracted with CH₂Cl₂; organic layer was washed with brine, dried over Na₂SO₄, and concentrated. The resulting oil was purified by HPLC, and used directly in the next step. The purified product (0.050 g, 0.12 mmol) was dissolved in MeOH (1.50 mL), treated with cyclobutanone (0.031 mL, 0.41 mmol), AcOH (0.015 mL, 0.25 mmol), and NaCNBH₃ (0.015 g, 0.25 mmol). The reaction mixture was allowed to stir at room temperature overnight, after which TLC and LC/MS indicated reaction was complete. The mixture was treated with NaOH (30%) until pH˜10, and was then concentrated under reduced pressure to rid of most of the MeOH. The resulting material was then diluted with CH₂Cl₂, washed with H₂O followed by brine, dried over Na₂SO₄, and concentrated under reduced pressure. This colorless oil was purified by silica gel chromatography to afford the title compound; MS (ES) m/z=482.2 [M+H]+.

### Example 24

### (3R)-3-{cyclobutyl[3-(5-fluoro-1H-indol-3-yl)propyl]amino}-8-(dimethylamino) chromane-5-carboxamide

Dimethylamine (2.0 M in THF; 10.00 mL, 20.00 mmol) was added to a flask under an inert atmosphere and was cooled to 0° C. Once cooled, n-BuLi (2.5 M in hexanes; 6.4 mL, 16.00 mmol) was added drop wise, and the mixture was allowed to stir for 0.5 hours at room temperature. After stirring was complete, the mixture was again cooled down to 0° C., and (3R)-8-fluoro-3-{[3-(5-fluoro-1H-indol-3-yl)propyl]amino}chromane-5-carboxamide (0.20 g, 0.52 mmol), which was dissolved in THF (1.00 mL), was added drop wise to the cooled reaction mixture. After stirring overnight, the reaction mixture was treated with NaHCO₃, and extracted with EtOAc. The organic extract was washed with brine, dried over NaSO₄, and concentrated to afford product as a brown oil which was used directly without purification. The crude product (0.200 g, 0.49 mmol) was dissolved in MeOH (6.00 mL), treated with cyclobutanone (0.13 mL, 1.73 mmol), AcOH (0.07 mL, 1.03 mmol), and NaCNBH₃ (0.07 g, 1.03 mmol). The reaction mixture was allowed to stir at room temperature overnight, after which TLC and LC/MS indicated reaction was complete. The mixture was treated with NaOH (30%) until pH˜10, and was then concentrated under reduced pressure to rid of most of the MeOH. The resulting material was then diluted with CH₂Cl₂, washed with H₂O followed by brine, dried over Na₂SO₄, and concentrated under reduced pressure. This brown oil was purified by HPLC to afford the title compound; MS (ES) m/z=465.3 [M+H]+.

### Example 25

### (3R)-3-{cyclobutyl[3-(5-fluoro-1H-indol-3-yl)propyl]amino}-8-(methylamino)-chromane-5-carboxamide

Methylamine (2.0 M in THF; 5.00 mL, 10.00 mmol) was added to a flask under an inert atmosphere and was then cooled to 0° C. Once cooled, n-BuLi (2.5 M in hexanes; 3.2 mL, 8.00 mmol) was added drop wise, and the mixture was allowed to stir for 0.5 hours at room temperature. After stirring was complete, the mixture was again cooled down to 0° C., and (3R)-8-fluoro-3-{[3-(5-fluoro-1H-indol-3-yl)propyl]amino}chromane-5-carboxamide (0.10 g, 0.26 mmol), which was dissolved in THF (0.50 mL), was added drop wise to the cooled reaction mixture. After stirring overnight, reaction mixture was treated with NaHCO₃, and extracted with EtOAc. The organic extract was washed with brine, dried over NaSO₄, and concentrated to afford product as a brown oil which was used directly without purification. The crude product (0.100 g, 0.25 mmol) was dissolved in MeOH (3.00 mL), treated with cyclobutanone (0.07 mL, 0.88 mmol), AcOH (0.03 mL, 0.53 mmol), and NaCNBH₃ (0.03 g, 0.53 mmol). The reaction mixture was allowed to stir at room temperature overnight, after which, TLC and LC/MS indicated reaction was complete. The mixture was treated with NaOH (30%) until pH˜10, and was then concentrated under reduced pressure to rid of most of the MeOH. The resulting material was then diluted with CH₂Cl₂, washed with H₂O followed by brine, dried over Na₂SO₄, and concentrated under reduced pressure. This brown oil was then subjected to silica gel chromatography to afford the title compound; MS (ES) m/z=451.2 [M+H]+, 473.2 [M+Na]+.

### Biological Assays

A protocol similar to that used by Cheetham et al. (Neuropharmacol., 1993, 32: 737) was used to determine the affinity of representative compounds described herein for the serotonin transporter. The compound's ability to displace ³H-paroxetine from male rat cortical membranes was determined using a Tom Tech filtration device to separate bound from free ³H-paroxetine and Wallac 1205 Beta Plate® counter to quantitate bound radioactivity. K is thus determined for standard clinical antidepressants are 1.96 nM for fluoxetine, 14.2 nM for imipramime and 67.6 nM for zimelidine. A strong correlation has been found between ³H-paroxetine binding in rat frontal cortex and ³H-serotonin uptake inhibition.

High affinity for the serotonin 5-HT_{1A} receptor was established by testing the claimed compound's ability to displace [³H] 8-OH-DPAT (dipropylaminotetralin) from the 5-HT_{1A} serotonin receptor following a modification of the procedure of Hall et al., (J. Neurochem., 1985, 44: 1685) which utilizes CHO cells stably transfected with human 5-HT_{1A} receptors. The 5-HT_{1A} affinities for representative compounds described herein are reported below as Kis.

The agonist or antagonist activity at 5-HT_{1A} receptors was established by using two different assays. The ³⁵S-GTPγS binding assay similar to that used by Lazareno and Birdsall (Br. J. Pharmacol., 1993, 109: 1120) was used to determine the test compound's ability to affect the binding of ³⁵S-GTPγS to membranes containing cloned human 5-HT_{1A} receptors. Agonists produce an increase in binding whereas antagonists produce no increase but rather reverse the effects of the standard agonist 8-OH-DPAT. The test compound's maximum stimulatory effect is represented as the Eₘₐₓ, while its potency is defined by the EC₅₀. The test compound's maximum inhibitory effect is represented as the Iₘₐₓ, while its potency is defined by the IC₅₀. The second assay measured cAMP accumulation upon binding of the ligand to the 5-HT_{1A} receptor. Antagonists block the effect of the standard agonist 8-OH-DPAT resulting in an increase in cAMP accumulation while agonists have the reverse effect. The test compound's maximum stimulatory or inhibitory effect is represented as the Eₘₐₓ while its potency is defined by either IC₅₀ for an antagonist or EC₅₀ for an agonist. [³H]-8-OH-DPAT was used to determine maximum agonist or antagonist response in both functional assays.

The results of the three standard experimental test procedures described above, as well as further details regarding the procedures, are provided below.

### Example 26

### 8-OH-DPAT Binding in CHO Cells Stably Transfected with Human 5HT1ₐ Receptor

Stabily transfected CHO cells were grown in DMEM containing 10% heat inactivated FBS and non-essential amino acids. Cells were scraped off the plate, transferred to centrifuge tubes, and washed twice by centrifugation (2000 rpm for 10 min., 4° C.) in buffer (50 mM Tris pH 7.5). The resulting pellets were aliquoted and placed at −80° C. On day of assay, the cells were thawed on ice, and resuspended in buffer. The binding assay was performed in a 96 well microtiter plate in a total volume of 250 mL.

Non-specific binding was determined in the presence of 10 mM 5HT, final ligand concentration is 1.5 nM. Following a 30 minute incubation at room temperature, the reaction was terminated by the addition of ice cold buffer and rapid filtration through a GF/B filter presoaked for 30 minutes in 0.5% PEI. Compounds were initially tested in a single point assay to determine percent inhibition at 1, 0.1, and 0.01 mM. Subsequently, Kᵢ values were determined for compounds defined to be active. Results are shown in Table 1.

### Example 27

### cAMP RIA in CHO Cell Stably Transfected with the h5HT1ₐ Receptor

Stabily transfected CHO cells were grown in DMEM containing 10% heat inactivated FBS and non-essential amino acids. The cells were plated at a density of 10(6) cells per well in a 24 well plate and incubated for 2 days in a CO₂ incubator. On the second day, the media was replaced with 0.5 ml treatment buffer (DMEM+25 mM HEPES, 5 mM theophylline, 10 uM pargyline) and incubated 10 minutes at 37° C.

Wells were treated with forskolin (1 μM final conc) followed immediately by test compound (0.1 and 1 uM for initial screen) and incubated for an additional 10 min at 37° C. The reaction was terminated by removal of the media and addition of 0.5 ml ice cold assay buffer (supplied in RIA kit). Plates were stored at −20° C. prior to assessment of cAMP formation by RIA.

Compounds shown to have no agonist activities were further analyzed for ability to reverse agonist activity. In separate experiments, 6 concentrations of antagonist were preincubated for 20 minutes prior to the addition of agonist and forskolin. Cells were harvested as described. The cAMP kit was supplied by Amersham and the RIA was performed as per kit instructions. Results are shown in Table 1.

### Example 28

### ³H-Paroxetine Binding to Assess Affinity of Drugs for the Serotonin Transporter

A protocol similar to that used by Cheetham et al. (Neuropharmacol. 32:737, 1993) was used to determine the affinity of compounds for the serotonin transporter. Briefly, frontal cortical membranes prepared from male S.D. rats were incubated with ³H-paroxetine (0.1 nM) for 60 min at 25° C. All tubes also contained either vehicle, test compound (one to eight concentrations), or a saturating concentration of fluoxetine (10 μM) to define specific binding. All reactions are terminated by the addition of ice cold Tris buffer followed by rapid filtration using a Tom Tech filtration device to separate bound from free ³H-paroxetine. Bound radioactivity was quantitated using a Wallac 1205 Beta Plate® counter. Nonlinear regression analysis was used to determine IC₅₀ values, which were converted to Kᵢ values using the method of Cheng and Prusoff (Biochem. Pharmacol. 22: 3099, 1973); Ki=IC50/((Radioligand conc.)/(1+KD)). Results are also shown in Table 1.

TABLE 1

| | | | | | 5-HT_{1A} |
|---|---|---|---|---|---|
| | 5-HT_{1A} | h5-HT | 5-HT_{1A} | 5-HT_{1A} | Function |
| | Receptor | Transporter | Function | Function | cAMP |
| Compound | Affinity | Function | GTPγS | GTPγS | EC₅₀ or IC₅₀ |
| (Example #) | Kᵢ (nM) | IC₅₀ (nM) | EC₅₀ (nM) | IC₅₀ (nM) | (nM) |
| 1 | 112.5 | 234 | 489 | Not Tested | EC₅₀ 230 |
| 2 | 10.4 | 13.2 | Not Tested | 119 | IC₅₀ 92.1 |
| 3 | 107.6 | 329 | Not Tested | 5033 | IC₅₀: 768 |
| 4 | 70.1 | 450 | Not Tested | 684 | EC₅₀ 151 |
| 5 | 35.3 | 214 | Not Tested | 3145 | EC₅₀ 118 |
| 6 | 136 | 347 | Not Tested | 2549 | EC₅₀ 312 |
| 7 | 636 | 2684 | Not Tested | Not Tested | Not Tested |
| 8 | 25.5 | 45.3 | Not Tested | 442 | IC₅₀ 439 |
| 9 | 515 | 34.0 | Not Tested | Not Tested | Not Tested |
| 10 | 20.5 | 8.0 | Not Tested | 321 | EC₅₀ 186 |
| 11 | 98.7 | 279 | Not Tested | 3030 | IC₅₀ 375 |
| 12 | Not Tested | 2581 | Not Tested | Not Tested | Not Tested |
| 13 | 214 | 339 | Not Tested | 2950 | Not Tested |
| 14 | 402 | 2.62 | Not Tested | 1372 | Not Tested |
| 15 | 1700 | 160 | Not Tested | Not Tested | Not Tested |
| 16 | 570 | 55 | Not Tested | 738 | EC₅₀ 323 |
| 17 | 662 | 5544 | Not Tested | Not Tested | Not Tested |
| 18 | 61.3 | 110 | Not Tested | Not Tested | IC₅₀ 94.2 |
| 19 | 43.6 | 407 | Not Tested | Not Tested | IC₅₀ 99.9 |
| 20 | 4.26 | 52.2 | Not Tested | 311 | EC₅₀ 51.8 |
| 21 | 5.93 | 90.4 | Not Tested | 724 | EC₅₀ 15.6 |
| 22 | 16.9 | 364 | Not Tested | 1184 | IC₅₀ 59.8 |
| 23 | 138 | 831 | Not Tested | Not Tested | IC₅₀ 137 |
| 24 | 72.7 | 125 | Not Tested | Not Tested | Not Tested |
| 25 | 51.5 | 137 | Not Tested | 3554 | IC₅₀: 140 |

Like the antidepressants fluoxetine, paroxetine and sertraline, the compounds described herein have the ability to block the reuptake of the brain neurotransmitter serotonin. They are thus useful for the treatment of diseases described herein, in particular, those commonly treated by the administration of serotonin selective reuptake inhibitor (SSRI) antidepressants, such as depression, (including but not limited to major depressive disorder, childhood depression and dysthymia), anxiety, panic disorder, post-traumatic stress disorder, premenstrual dysphoric disorder (also known as premenstrual syndrome), attention deficit disorder (with and without hyperactivity), obsessive compulsive disorder, social anxiety disorder, generalized anxiety disorder, obesity, eating disorders such as anorexia nervosa, bulimia nervosa, vasomotor flushing, cocaine and alcohol addiction, sexual dysfunction, and related illnesses. Moreover, some of the compounds described herein have potent affinity for and antagonist activity at brain 5-HT_{1A} serotonin receptors. Clinical trials employing drug mixtures (e.g., fluoxetine and pindolol) have demonstrated a more rapid onset of antidepressant efficacy for a treatment combining SSRI activity and 5-HT_{1A} antagonism (Blier and Bergeron, J. Clin. Psychopharmacol., 1995, 15(3): 217-22; F. Artigas et al., Trends Neurosci., 1996, 19(9): 378-83; Tome et al., J. Affect Disord., 1997, 44(2-3): 101-9). The compounds described herein are thus interesting and useful for treating depressive illnesses, as well as other disease states described herein.

When ranges are used herein for physical properties, such as molecular weight, or chemical properties, such as chemical Formulae, all combination and subcombinations of ranges of specific embodiments therein are intended to be included.

The disclosures of each patent, patent application, and publication cited or described in this document are hereby incorporated herein by reference, in their entirety.

Those skilled in the art will appreciate that numerous changes and modifications can be made to the embodiments disclosed herein and that such changes and modifications can be made without departing from the spirit of the invention. It is, therefore, intended that the appended claims cover all such equivalent variations as fall within the true spirit and scope of the invention.

We claim:

1. A compound of Formula I: 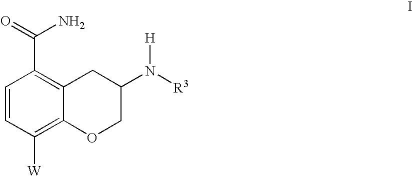
or an enantiomer, diastereomer, tautomer, or pharmaceutically acceptable salt or solvate thereof, wherein:
X is (C₁-C₆)-alkyl, —OR¹², —SR¹², or —NR¹³R¹⁴;
R¹ is hydrogen, (C₁-C₃)-alkyl, cycloalkyl, cycloalkylalkyl, phenyl, or benzyl;
R² is hydrogen, (C₁-C₄)-alkyl, (C₃-C₆)-cycloalkyl, cycloalkylalkyl, aralkyl, or haloalkyl;
R³ is —(CH₂)ₙ—R⁴, 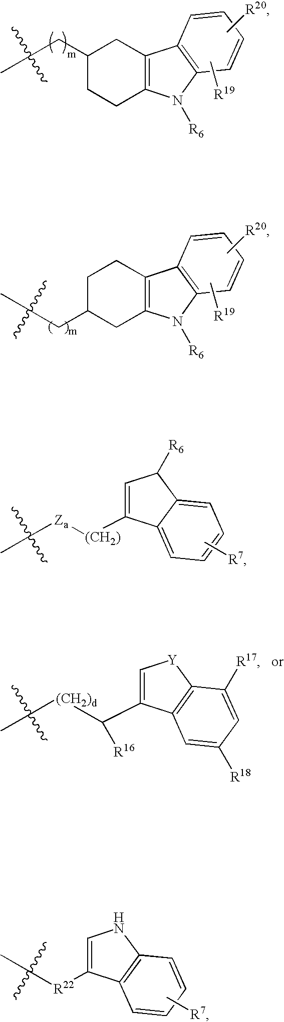
wherein n is an integer of 2 to 4; and R⁴ is A, B, C, D, E, G, J, K, L, P, U, AA or BB, provided that when n is an integer of 2, then R⁴ is A, B, C, D, E, G, J, K, L, U, AA or BB; and further provided that when n is an integer of 3 or 4, then R⁴ is A, B, C, D, K, L, P, U, AA or BB; 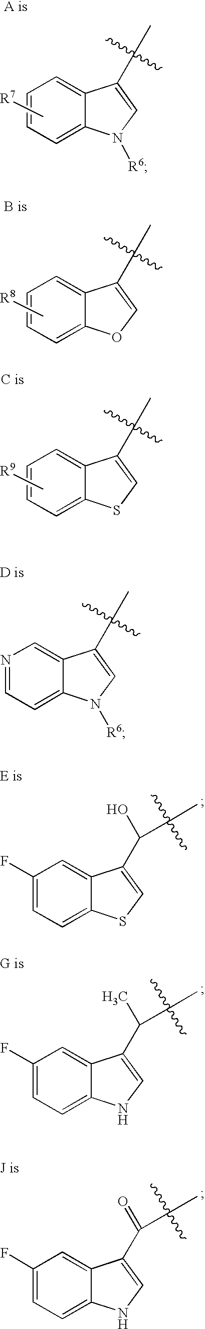 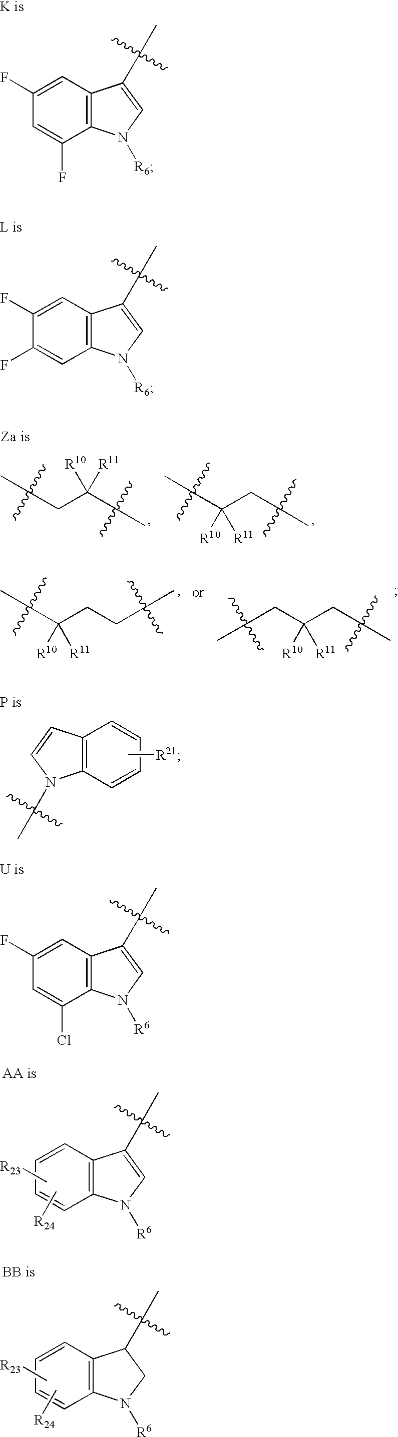
R⁶ is hydrogen or (C₁-C₆)-alkyl;
R⁷ is hydrogen, fluoro, chloro, cyano, or alkoxy;
R⁸ is hydrogen, halo, (C₁-C₃)-alkoxy or (C₁-C₃)-alkyl;
R⁹ is hydrogen, halo, (C₁-C₃)-alkoxy or (C₁-C₃)-alkyl;
R¹⁰ is hydrogen or methyl;
R¹¹ is methyl;
R¹², R¹³ and R¹⁴ are each independently hydrogen or (C₁-C₆)-alkyl, or R¹³ and R¹⁴ together with the N to which they are bonded optionally form a heterocycle;
R¹⁹ and R²⁰ are independently hydrogen, fluoro, chloro, cyano, or (C₁-C₆)-alkyl;
R²¹ is hydrogen or fluoro;
R²² is a 3- to 7-membered saturated carbocyclic ring;
R²³ and R²⁴ are independently hydrogen, halogen, cyano, or (C₁-C₆)-alkyl;
m is an integer of 1 or 2;
Y is O, S, or NH;
provided that when Y is O, then R¹⁶ is hydrogen; R¹⁷ is hydrogen or OCH₃; R¹⁸ is hydrogen; and d is an integer of 2 or 3;
further provided that when Y is S, then R¹⁶ is hydrogen or hydroxyl; R¹⁷ is hydrogen; R¹⁸ is hydrogen or fluoro; and d is an integer of 2;
further provided that when Y is NH, then R¹⁶ is keto (═O) or methyl; R¹⁷ is hydrogen; R¹⁸ is fluoro; and d is an integer of 2.

2. The compound of claim 1, wherein
R³ is —(CH₂)ₙ—R⁴; and
R⁴ is 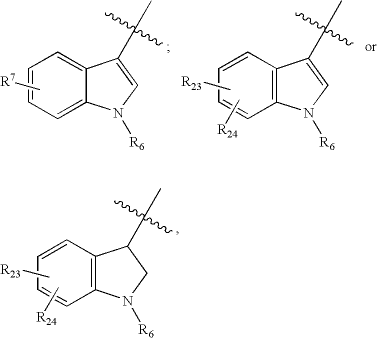
in which R⁶,
R⁷, R²³ and R²⁴ are as defined in claim 1.

3. The compound of claim 1 wherein formula I has the structure of Formula II or III: 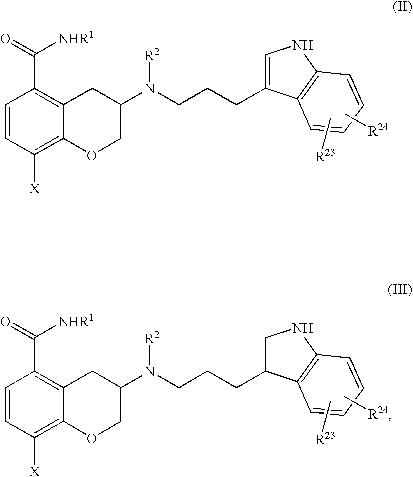
wherein X, R¹, R², R²³ and R²⁴ are as defined in claim 1.

4. The compound of claim 1 of Formula IV or V: 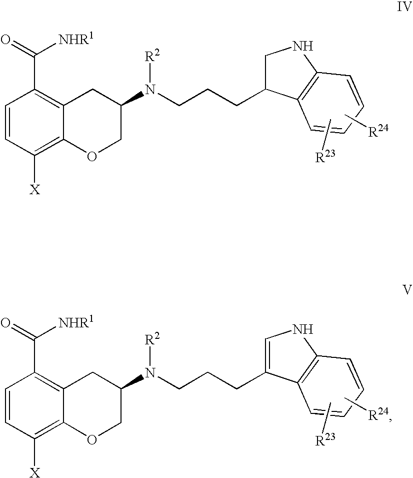
wherein X, R¹, R², R²³ and R²⁴ are as defined in claim 1, or a tautomer, or pharmaceutically acceptable salt or solvate thereof.

5. The compound of claim 1, wherein X is (C₁-C₃)-alkyl, —OR¹², —SR¹², or —NR¹³R¹⁴; and R¹², R¹³, and R¹⁴ are each independently hydrogen or (C₁-C₃)alkyl, or R¹³ and R¹⁴ together with the N to which they are bonded optionally form a substituted or unsubstituted 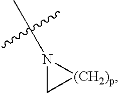
in which p is an integer selected from the group consisting of 1, 2, 3, 4 and 5.

6. The compound of claim 1, wherein X is (C₁-C₃)-alkyl, —OH, —OCH₃, —OCH₂CH₃, —OCH₂CH₂CH₃, —OCH(CH₃)₂, —SCH₃, —SCH₂CH₃, —NHCH₃, —NHCH₂CH₃, —N(CH₃)₂, —N(CH₃)(CH₂CH₃), or —N(CH₂CH₃)₂.

7. The compound of claim 1, wherein R¹ is hydrogen or (C₁-C₃)-alkyl.

8. The compound of claim 1, wherein R² is hydrogen, (C₁-C₄)-alkyl, (C₃-C₆)-cycloalkyl, cycloalkylalkyl, or haloalkyl.

9. The compound of claim 1, wherein R² is hydrogen, methyl, ethyl, propyl, isopropyl, butyl, isobutyl, cyclobutyl, cyclopropyl, trifluoropropyl, trifluoroethyl, trifluorobutyl, or cyclopropylmethyl.

10. The compound of claim 1, wherein R⁷ is hydrogen, fluoro, or chloro.

11. The compound of claim 1, wherein R²³ and R²⁴ are each independently hydrogen, fluoro, chloro, bromo, or iodo.

12. The compound of claim 1, wherein R²³ and R²⁴ are each independently hydrogen or fluoro.

13. The compound of claim 1, wherein R⁶ is hydrogen.

14. A compound according to claim 1, wherein said compound is:
3-{[3-(5-fluoro-1H-indol-3-yl)propyl]amino}-8-methoxychromane-5-carboxamide;
(+)-3-{[3-(5-fluoro-1H-indol-3-yl)propyl]amino}-8-methoxychromane-5-carboxamide;
(−)-3-{[3-(5-fluoro-1H-indol-3-yl)propyl]amino}-8-methoxychromane-5-carboxamide;
3-{cyclobutyl[3-(5-fluoro-1H-indol-3-yl)propyl]amino}-8-methoxychromane-5-carboxamide
(+)-3-{cyclobutyl[3-(5-fluoro-1H-indol-3-yl)propyl]amino}-8-methoxychromane-5-carboxamide;
(−)₃-{cyclobutyl[3-(5-fluoro-1H-indol-3-yl)propyl]amino}-8-methoxychromane-5-carboxamide;
3-{cyclopropylmethyl[3-(5-fluoro-1H-indol-3-yl)propyl]amino}-8-methoxychromane-5-carboxamide;
(−)-3-{cyclopropylmethyl[3-(5-fluoro-1H-indol-3-yl)propyl]amino}-8-methoxy chromane-5-carboxamide;
(+)-3-{cyclopropylmethyl[3-(5-fluoro-1H-indol-3-yl)propyl]amino}-8-methoxy chromane-5-carboxamide;
3-{cyclobutyl[3-(5-fluoro-1H-indol-3-yl)propyl]amino}-8-methylchromane-5-carboxamide;
(+)-3-{cyclobutyl[3-(5-fluoro-1H-indol-3-yl)propyl]amino}-8-methylchromane-5-carboxamide;
(−)-3-{cyclobutyl[3-(5-fluoro-1H-indol-3-yl)propyl]amino}-8-methylchromane-5-carboxamide;
3-{cyclobutyl[3-(5-fluoro-1H-indol-3-yl)propyl]amino}-8-ethylchromane-5-carboxamide;
(+)-3-{cyclobutyl[3-(5-fluoro-1H-indol-3-yl)propyl]amino}-8-ethylchromane-5-carboxamide;
(−)-3-{cyclobutyl[3-(5-fluoro-1H-indol-3-yl)propyl]amino}-8-ethylchromane-5-carboxamide;
3-{cyclobutyl[3-(5,7-difluoro-1H-indol-3-yl)propyl]amino}-8-methoxychromane-5-carboxamide;
(−)-3-{cyclobutyl[3-(5,7-difluoro-1H-indol-3-yl)propyl]amino}-8-methoxychromane-5-carboxamide;
(+)-3-{cyclobutyl[3-(5,7-difluoro-1H-indol-3-yl)propyl]amino}-8-methoxychromane-5-carboxamide;
3-{[3-(5-fluoro-1H-indol-3-yl)propyl](propyl)amino}-8-methoxychromane-5-carboxamide;
(+)-3-{[3-(5-fluoro-1H-indol-3-yl)propyl](propyl)amino}-8-methoxychromane-5-carboxamide;
(−)-3-{[3-(5-fluoro-1H-indol-3-yl)propyl](propyl)amino}-8-methoxychromane-5-carboxamide;
3-{cyclobutyl[3-(5-fluoro-1H-indol-3-yl)propyl]amino}-8-methylaminochromane-5-carboxamide;
3-{cyclobutyl[3-(5-fluoro-1H-indol-3-yl)propyl]amino}-8-(methylthio)chromane-5-carboxamide;
3-{cyclobutyl[3-(5-fluoro-1H-indol-3-yl)propyl]amino}-8-dimethylaminochromane-5-carboxamide;
3-{ethyl[3-(5-fluoro-1H-indol-3-yl)propyl]amino}-8-methoxychromane-5-carboxamide;
3-{[3-(5-fluoro-1H-indolin-3-yl)propyl]amino}-8-methoxychromane-5-carboxamide;
(+)-3-{[3-(5-fluoro-1H-indolin-3-yl)propyl]amino}-8-methoxychromane-5-carboxamide;
(−)-3-{[3-(5-fluoro-1H-indolin-3-yl)propyl]amino}-8-methoxychromane-5-carboxamide;
3-{cyclobutyl[3-(5-fluoro-1H-indolin-3-yl)propyl]amino}-8-methoxychromane-5-carboxamide;
(+)-3-{cyclobutyl[3-(5-fluoro-1H-indolin-3-yl)propyl]amino)-8-methoxychromane-5-carboxamide;
(−)₃-{cyclobutyl[3-(5-fluoro-1H-indolin-3-yl)propyl]amino)-8-methoxychromane-5-carboxamide;
3-{cyclopropylmethyl[3-(5-fluoro-1H-indolin-3-yl)propyl]amino}-8-methoxychromane-5-carboxamide;
(−)-3-{cyclopropylmethyl[3-(5-fluoro-1H-indolin-3-yl)propyl]amino}-8-methoxy chromane-5-carboxamide;
(+)-3-{cyclopropylmethyl[3-(5-fluoro-1H-indolin-3-yl)propyl]amino}-8-methoxy chromane-5-carboxamide;
3-{cyclobutyl[3-(5-fluoro-1H-indolin-3-yl)propyl]amino}-8-methylchromane-5-carboxamide;
(+)-3-{cyclobutyl[3-(5-fluoro-1H-indolin-3-yl)propyl]amino}-8-methylchromane-5-carboxamide;
(−)-3-{cyclobutyl[3-(5-fluoro-1H-indolin-3-yl)propyl]amino}-8-methylchromane-5-carboxamide;
3-{cyclobutyl[3-(5-fluoro-1H-indolin-3-yl)propyl]amino}-8-ethylchromane-5-carboxamide;
(+)-3-{cyclobutyl[3-(5-fluoro-1H-indolin-3-yl)propyl]amino}-8-ethylchromane-5-carboxamide;
(−)-3-{cyclobutyl[3-(5-fluoro-1H-indolin-3-yl)propyl]amino}-8-ethylchromane-5-carboxamide;
3-{cyclobutyl[3-(5,7-difluoro-1H-indolin-3-yl)propyl]amino}-8-methoxychromane-5-carboxamide;
(−)-3-{cyclobutyl[3-(5,7-difluoro-1H-indolin-3-yl)propyl]amino}-8-methoxychromane-5-carboxamide;
(+)-3-{cyclobutyl[3-(5,7-difluoro-1H-indolin-3-yl)propyl]amino}-8-methoxychromane-5-carboxamide;
3-{[4-(5-fluoro-1H-indolin-3-yl)-1-propyl](propyl)amino}-8-methoxychromane-5-carboxamide;
(+)-3-{[4-(5-fluoro-1H-indolin-3-yl)-1-propyl](propyl)amino}-8-methoxy-chromane-5-carboxamide;
(−)-3-{[4-(5-fluoro-1H-indolin-3-yl)-1-propyl](propyl)amino}-8-methoxychromane-5-carboxamide;
3-{cyclobutyl[3-(5-fluoro-1H-indolin-3-yl)propyl]amino}-8-methylaminochromane-5-carboxamide;
(+)-3-{cyclobutyl[3-(5-fluoro-1H-indolin-3-yl)propyl]amino}-8-methylaminochromane-5-carboxamide;
(−)-3-{cyclobutyl[3-(5-fluoro-1H-indolin-3-yl)propyl]amino}-8-methylaminochromane-5-carboxamide;
3-{cyclobutyl[3-(5-fluoro-1H-indolin-3-yl)propyl]amino}-(8-methylthio)-chromane-5-carboxamide;
3-{cyclobutyl[3-(5-fluoro-1H-indolin-3-yl)propyl]amino}-(8-methylthio)-chromane-5-carboxamide;
(+)-3-{cyclobutyl[3-(5-fluoro-1H-indolin-3-yl)propyl]amino}-(8-methylthio)-chromane-5-carboxamide;
(−)-3-{cyclobutyl[3-(5-fluoro-1H-indolin-3-yl)propyl]amino}-(8-methylthio)-chromane-5-carboxamide;
3-{cyclobutyl[3-(5-fluoro-1H-indolin-3-yl)propyl]amino}-8-dimethylaminochromane-5-carboxamide;
(+)-3-{cyclobutyl[3-(5-fluoro-1H-indolin-3-yl)propyl]amino}-8-dimethylaminochromane-5-carboxamide;
(−)-3-{cyclobutyl[3-(5-fluoro-1H-indolin-3-yl)propyl]amino}-8-dimethylaminochromane-5-carboxamide;
3-{ethyl[3-(5-fluoro-1H-indolin-3-yl)propyl]amino}-8-methoxychromane-5-carboxamide;
(+)-3-{ethyl[3-(5-fluoro-1H-indolin-3-yl)propyl]amino}-8-methoxychromane-5-carboxamide;
(−)-3-{ethyl[3-(5-fluoro-1H-indolin-3-yl)propyl]amino}-8-methoxychromane-5-carboxamide;
3-{cyclobutyl[3-(5-fluoro-1H-indol-3-yl)propyl]amino}-8-ethoxychromane-5-carboxamide;
(3R)-3-{cyclobutyl[3-(5-fluoro-1H-indol-3-yl)propyl]amino}-8-ethoxychromane-5-carboxamide;
(+)-3-{cyclobutyl[3-(5-fluoro-1H-indol-3-yl)propyl]amino}-8-ethoxychromane-5-carboxamide;
(−)-3-{cyclobutyl[3-(5-fluoro-1H-indol-3-yl)propyl]amino}-8-ethoxychromane-5-carboxamide;
3-{cyclobutyl[3-(5-fluoro-1H-indol-3-yl)propyl]amino}-8-propoxychromane-5-carboxamide;
(3R)-3-{cyclobutyl[3-(5-fluoro-1H-indol-3-yl)propyl]amino}-8-propoxychromane-5-carboxamide;
(+)-3-{cyclobutyl[3-(5-fluoro-1H-indol-3-yl)propyl]amino}-8-propoxychromane-5-carboxamide;
(−)-3-{cyclobutyl[3-(5-fluoro-1H-indol-3-yl)propyl]amino}-8-propoxychromane-5-carboxamide;
3-{cyclobutyl[3-(5-fluoro-1H-indol-3-yl)propyl]amino}-8-isopropoxychromane-5-carboxamide;
(+)-3-{cyclobutyl[3-(5-fluoro-1H-indol-3-yl)propyl]amino}-8-isopropoxychromane-5-carboxamide;
(−)-3-{cyclobutyl[3-(5-fluoro-1H-indol-3-yl)propyl]amino}-8-isopropoxychromane-5-carboxamide;
3-{cyclobutyl[3-(5-fluoro-1H-indol-3-yl)propyl]amino}-8-isopropoxychromane-5-carboxamide;
(3R)-3-{cyclobutyl[3-(5-fluoro-1H-indol-3-yl)propyl]amino}-8-isopropoxychromane-5-carboxamide;
(+)-3-{cyclobutyl[3-(5-fluoro-1H-indol-3-yl)propyl]amino}-8-isopropoxychromane-5-carboxamide;
(−)-3-{cyclobutyl[3-(5-fluoro-1H-indol-3-yl)propyl]amino}-8-isopropoxychromane-5-carboxamide;
3-{cyclobutyl[3-(5-fluoro-1H-indol-3-yl)propyl]amino}-8-hydroxychromane-5-carboxamide;
(3R)-3-{cyclobutyl[3-(5-fluoro-1H-indol-3-yl)propyl]amino}-8-hydroxychromane-5-carboxamide;
(+)-3-{cyclobutyl[3-(5-fluoro-1H-indol-3-yl)propyl]amino}-8-hydroxychromane-5-carboxamide;
(−)-3-{cyclobutyl[3-(5-fluoro-1H-indol-3-yl)propyl]amino}-8-hydroxychromane-5-carboxamide;
3-{(3,3,3-trifluoropropyl)-[3-(5-fluoro-1H-indol-3-yl)propyl]amino}-8-methoxychromane-5-carboxamide;
(3R)-3-{(3,3,3-trifluoropropyl)-[3-(5-fluoro-1H-indol-3-yl)propyl]amino}-8-methoxychromane-5-carboxamide;
(+)-3-{(3,3,3-trifluoropropyl)-[3-(5-fluoro-1H-indol-3-yl)propyl]amino}-8-methoxychromane-5-carboxamide;
(−)-3-{(3,3,3-trifluoropropyl)-[3-(5-fluoro-1H-indol-3-yl)propyl]amino}-8-methoxychromane-5-carboxamide;
3-{isopropyl[3-(5-fluoro-1H-indol-3-yl)propyl]amino}-8-methoxychromane-5-carboxamide;
(3R)-3-{isopropyl[3-(5-fluoro-1H-indol-3-yl)propyl]amino}-8-methoxychromane-5-carboxamide;
(+)-3-{isopropyl[3-(5-fluoro-1H-indol-3-yl)propyl]amino}-8-methoxychromane-5-carboxamide;
(−)-3-{isopropyl[3-(5-fluoro-1H-indol-3-yl)propyl]amino}-8-methoxychromane-5-carboxamide;
-3-{butyl[3-(5-fluoro-1H-indol-3-yl)propyl]amino}-8-methoxychromane-5-carboxamide;
(3R)-3-{butyl[3-(5-fluoro-1H-indol-3-yl)propyl]amino}-8-methoxychromane-5-carboxamide;
(+)-3-{butyl[3-(5-fluoro-1H-indol-3-yl)propyl]amino}-8-methoxychromane-5-carboxamide;
(−)-3-{butyl[3-(5-fluoro-1H-indol-3-yl)propyl]amino}-8-methoxychromane-5-carboxamide;
(3R)-3-{4,4,4-trifluorobutyl[3-(5-fluoro-1H-indol-3-yl)propyl]amino}-8-methoxychromane-5-carboxamide;
3-{2-isobutyl[3-(5-fluoro-1H-indol-3-yl)propyl]amino}-8-methoxychromane-5-carboxamide;
(3R)-3-{2-isobutyl[3-(5-fluoro-1H-indol-3-yl)propyl]amino}-8-methoxychromane-5-carboxamide;
(+)-3-{2-isobutyl[3-(5-fluoro-1H-indol-3-yl)propyl]amino}-8-methoxychromane-5-carboxamide;
(−)-3-{2-isobutyl[3-(5-fluoro-1H-indol-3-yl)propyl]amino}-8-methoxychromane-5-carboxamide;
3-{methyl[3-(5-fluoro-1H-indol-3-yl)propyl]amino}-8-methoxychromane-5-carboxamide;
(3R)-3-{methyl[3-(5-fluoro-1H-indol-3-yl)propyl]amino}-8-methoxychromane-5-carboxamide;
(+)-3-{methyl[3-(5-fluoro-1H-indol-3-yl)propyl]amino}-8-methoxychromane-5-carboxamide;
(−)-3-{methyl[3-(5-fluoro-1H-indol-3-yl)propyl]amino}-8-methoxychromane-5-carboxamide;
3-{cyclobutyl[3-(5-fluoro-1H-indol-3-yl)propyl]amino}-8-methoxychromane-5-methylcarboxamide;
(3R)-3-{cyclobutyl[3-(5-fluoro-1H-indol-3-yl)propyl]amino}-8-methoxychromane-5-methylcarboxamide;
(+)-3-{cyclobutyl[3-(5-fluoro-1H-indol-3-yl)propyl]amino}-8-methoxychromane-5-methylcarboxamide;
(−)-3-{cyclobutyl[3-(5-fluoro-1H-indol-3-yl)propyl]amino}-8-methoxychromane-5-methylcarboxamide;
3-{(3,3,3-trifluoropropyl)-[3-(5-fluoro-1H-indol-3-yl)propyl]amino}-8-methoxychromane-5-methylcarboxamide;
(3R)-3-{(3,3,3-trifluoropropyl)-[3-(5-fluoro-1H-indol-3-yl)propyl]amino}-8-methoxychromane-5-methylcarboxamide;
(+)-3-{(3,3,3-trifluoropropyl)-[3-(5-fluoro-1H-indol-3-yl)propyl]amino}-8-methoxychromane-5-methylcarboxamide;
(−)-3-{(3,3,3-trifluoropropyl)-[3-(5-fluoro-1H-indol-3-yl)propyl]amino}-8-methoxychromane-5-methylcarboxamide;
3-{cyclobutyl[3-(5-fluoro-1H-indol-3-yl)propyl]amino}-8-(ethylthio)chromane-5-carboxamide;
(3R)-3-{cyclobutyl[3-(5-fluoro-1H-indol-3-yl)propyl]amino}-8-(ethylthio)chromane-5-carboxamide;
(+)-3-{cyclobutyl[3-(5-fluoro-1H-indol-3-yl)propyl]amino}-8-(ethylthio)chromane-5-carboxamide; and
(−)-3-{cyclobutyl[3-(5-fluoro-1H-indol-3-yl)propyl]amino}-8-(ethylthio)chromane-5-carboxamide;
or a pharmaceutically acceptable salt thereof.

15. A pharmaceutical composition comprising a compound of claim 1 and one or more pharmaceutically acceptable carriers.

16. A method of synthesizing a compound comprising:
(a) reacting a compound of Formula 1: 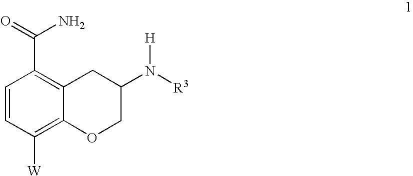
wherein
W is halogen; and
R³ is —(CH₂)ₙ—R⁴, 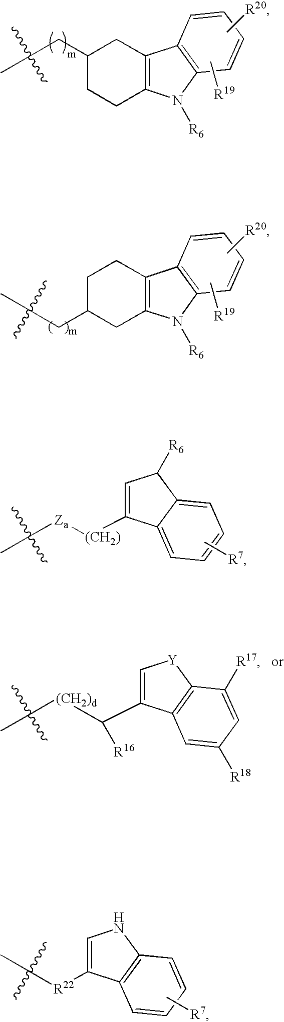
wherein n is an integer of 2 to 4; and R⁴ is A, B, C, D, E, G, J, K, L, P, U, AA or BB, provided that when n is an integer of 2, then R⁴ is A, B, C, D, E, G, J, K, L, U, AA or BB; and further provided that when n is an integer of 3 or 4, then R⁴ is A, B, C, D, K, L, P, U, AA or BB; 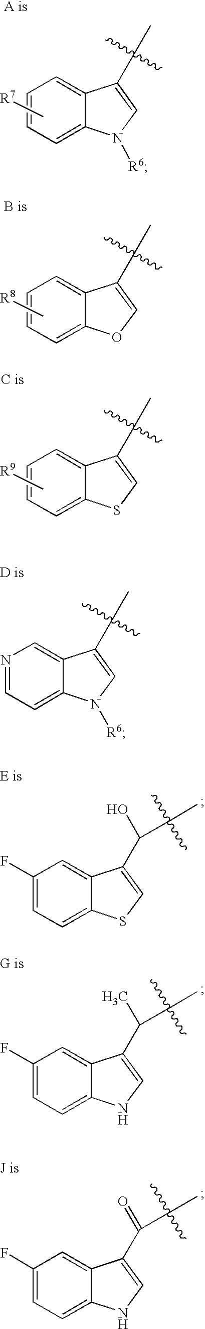 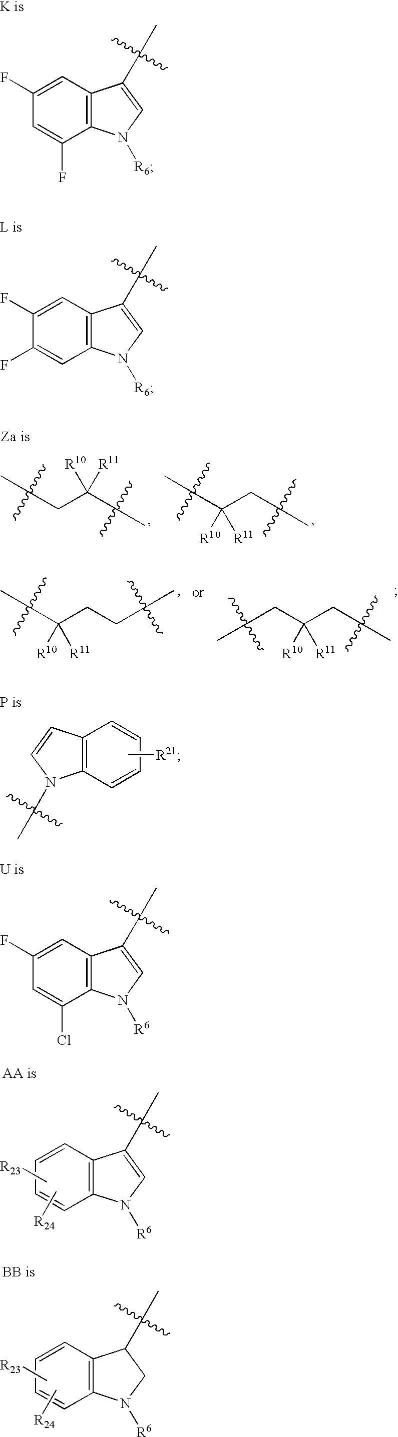
R⁶ is hydrogen or (C₁-C₆)-alkyl;
R⁷ is hydrogen, fluoro, chloro, cyano, or alkoxy;
R⁸ is hydrogen, halo, (C₁-C₃)-alkoxy or (C₁-C₃)-alkyl;
R⁹ is hydrogen, halo, (C₁-C₃)-alkoxy or (C₁-C₃)-alkyl;
R¹⁰ is hydrogen or methyl;
R¹¹ is methyl;
R¹⁹ and R²⁰ are independently hydrogen, fluoro, chloro, cyano, or (C₁-C₆)-alkyl;
R²¹ is hydrogen or fluoro;
R²² is a 3- to 7-membered saturated carbocyclic ring;
R²³ and R²⁴ are independently hydrogen, halogen, cyano, or (C₁-C₆)-alkyl;
m is an integer of 1 or 2;
Y is O, S, or NH;
provided that when Y is O, then R¹⁶ is hydrogen; R¹⁷ is hydrogen or OCH₃; R¹⁸ is hydrogen;
and d is an integer of 2 or 3;
further provided that when Y is S, then R¹⁶ is hydrogen or hydroxyl; R¹⁷ is hydrogen; R¹⁸ is hydrogen or fluoro; and d is an integer of 2;
further provided that when Y is NH, then R¹⁶ is keto (═O) or methyl; R¹⁷ is hydrogen; R¹⁸ is fluoro; and d is an integer of 2;
with an alkyl alcohol salt of Formula Ia:
R¹²O⁻Z⁺ (1a)
wherein R¹² is (C₁-C₆)-alkyl and
Z is a pharmaceutically acceptable counter ion;
under conditions effective to produce a compound of Formula 2: 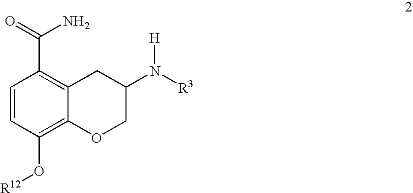
(b) optionally converting the compound of Formula 2 to a compound of Formula 3: 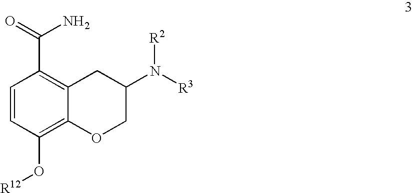
wherein R² is (C₁-C₄)-alkyl, (C₃-C₆)-cycloalkyl, cycloalkylalkyl, aralkyl, or haloalkyl;
under conditions effective to bring about reductive amination at the basic nitrogen of the compound of Formula 2; and
(c) further optionally reacting the compound of Formula 3 under conditions effective to remove the R¹² group, thereby providing a compound of Formula 4:

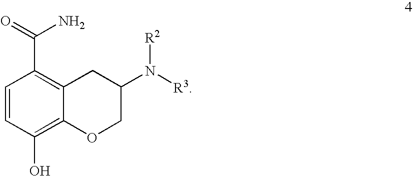

17. The method of claim 16, further comprising:
(a) reacting the compound of Formula 2, 3 or 4 under conditions effective to bring about hydrolysis of the amino group to provide a compound of Formula 8: 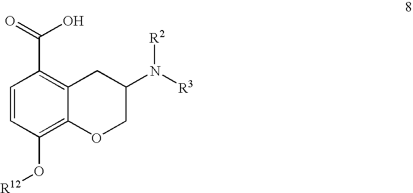
wherein R² is hydrogen, (C₁-C₄)-alkyl, (C₃-C₆)-cycloalkyl, cycloalkylalkyl, aralkyl, or haloalkyl; R³ is as defined claim 16; and R¹² is hydrogen or (C₁-C₆)-alkyl;
(b) reacting the compound of Formula 8 with an alkylamine of Formula 8a:
R¹NH₂ (8a)
wherein R¹ is hydrogen or (C₁-C₃)-alkyl;
under conditions effective to provide a compound of Formula Ia: 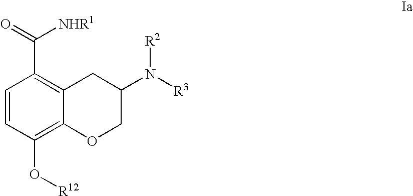
wherein R¹, R², R³, and R¹² are as defined hereinabove.

18. The method of claim 16, further comprising:
(a) reacting the compound of Formula 4 under conditions effective to produce a compound of Formula 5 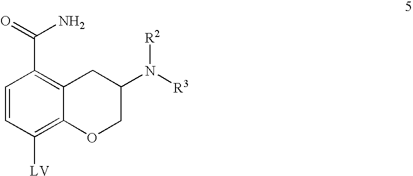
wherein LV is a precursor for a palladium catalyzed reaction;
(b) reacting a compound of Formula 5 under conditions effective to product a compound of Formula 7: 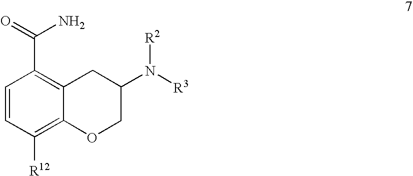
wherein R¹² is (C₁-C₆)-alkyl;
(c) reacting the compound of Formula 7 under conditions effective to bring about hydrolysis of the amino group to provide a compound of Formula 8: 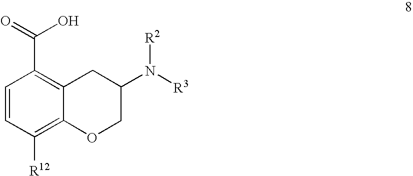
(d) reacting the compound of Formula 8 with an alkylamine of Formula 8a:
R¹NH₂ (8a)
wherein R¹ is hydrogen or (C₁-C₃)-alkyl;
under conditions effective to provide a compound of Formula Ib:

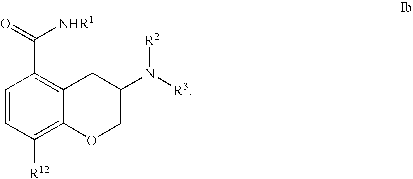

19. A method of synthesizing a compound comprising:
(a) reacting a compound of Formula 1: 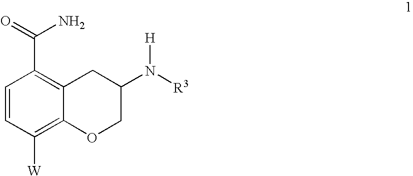
wherein
W is halogen; and
R³ is —(CH₂)ₙ—R⁴, 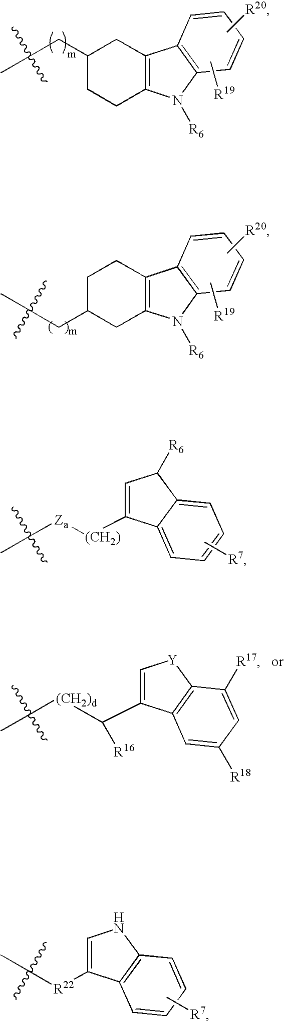
wherein n is an integer of 2 to 4; and R⁴ is A, B, C, D, E, G, J, K, L, P, U, AA or BB, provided that when n is an integer of 2, then R⁴ is A, B, C, D, E, G, J, K, L, U, AA or BB; and further provided that when n is an integer of 3 or 4, then R⁴ is A, B, C, D, K, L, P, U, AA or BB; 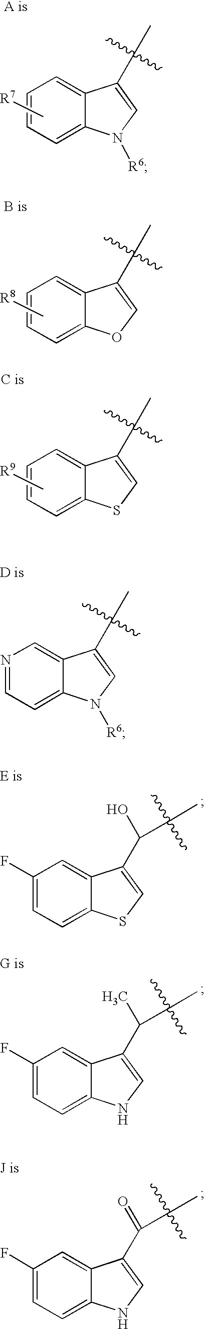 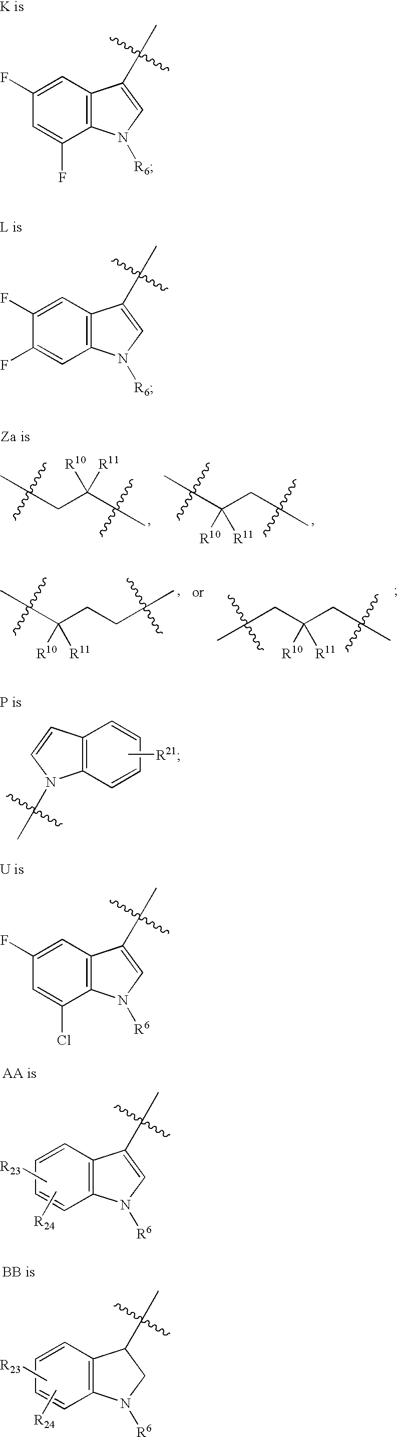
R⁶ is hydrogen or (C₁-C₆)-alkyl;
R⁷ is hydrogen, fluoro, chloro, cyano, or alkoxy;
R⁸ is hydrogen, halo, (C₁-C₃)-alkoxy or (C₁-C₃)-alkyl;
R⁹ is hydrogen, halo, (C₁-C₃)-alkoxy or (C₁-C₃)-alkyl;
R¹⁰ is hydrogen or methyl;
R¹¹ is methyl;
R¹⁹ and R²⁰ are independently hydrogen, fluoro, chloro, cyano, or (C₁-C₆)-alkyl;
R²¹ is hydrogen or fluoro;
R²² is a 3- to 7-membered saturated carbocyclic ring;
R²³ and R²⁴ are independently hydrogen, halogen, cyano, or (C₁-C₆)-alkyl;
m is an integer of 1 or 2;
Y is O, S, or NH;
provided that when Y is O, then R¹⁶ is hydrogen; R¹⁷ is hydrogen or OCH₃; R¹⁸ is hydrogen;
and d is an integer of 2 or 3;
further provided that when Y is S, then R¹⁶ is hydrogen or hydroxyl; R¹⁷ is hydrogen; R¹⁸ is hydrogen or fluoro; and d is an integer of 2;
further provided that when Y is NH, then R¹⁶ is keto (═O) or methyl; R¹⁷ is hydrogen; R¹⁸ is fluoro; and d is an integer of 2;
with an alkylthio salt of Formula Ia or an amine salt of Formula 1b:
R¹²S⁻Z⁺ (1a)
or R¹³R¹⁴N⁻Z⁺ (1b)
wherein R¹², R¹³, and R¹⁴ are each independently hydrogen or (C₁-C₆)-alkyl, and Z is a pharmaceutically acceptable counter ion;
under conditions effective to produce a compound of Formula 6: 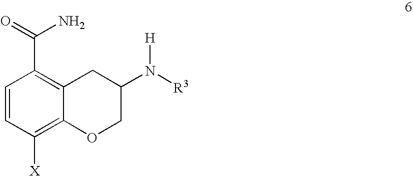
wherein X is —SR¹² or —NR¹³R¹⁴;
(b) reacting the compound of Formula 6 with an alkylaldehyde of Formula 6a or a ketone of Formula 6b:
R²C(O)H (6a);
R²C(O)R² (6b);
wherein R² is hydrogen, (C₁-C₄)-alkyl, (C₃-C₆)-cycloalkyl, cycloalkylalkyl, aralkyl, or haloalkyl;
under conditions effective to bring about reductive alkylation at the basic nitrogen to product a compound of Formula 7: 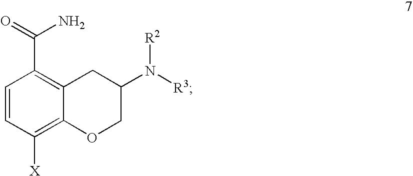
(c) reacting the compound of Formula 7 under conditions effective to bring about hydrolysis of the amino group to provide a compound of Formula 8: 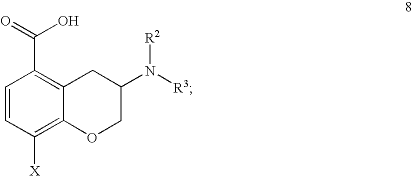
(d) reacting the compound of Formula 8 with an alkylamine of Formula 8a:
R¹NH₂ (8a)
wherein R¹ is hydrogen or (C₁-C₃)-alkyl;
under conditions effective to provide a compound of Formula Ic:

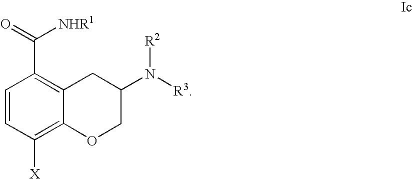

20. The method of claim 16, wherein W is —F, —Cl, or —Br.

21. The method of claim 16, wherein R¹² is (C₁-C₃)-alkyl.

22. The method of claim 16, wherein Z is Na⁺, K⁺, or Li⁺.

23. The method of claim 18, wherein LV is triflate.

24. A method of synthesizing a compound comprising:
(a) reacting a compound of Formula 10: 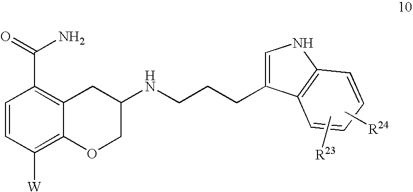
wherein W is halogen:
R²³ and R²⁴ are each independently hydrogen, —F, —Cl, —Br, —I, —CN, or (C₁-C₆)-alkyl;
with an alkyl alcohol salt of Formula Ia:
R¹²O⁻Z⁺ (1a)
wherein R¹² is (C₁-C₆)-alkyl and Z is a pharmaceutically acceptable counter ion;
under conditions effective to produce a compound of Formula 11: 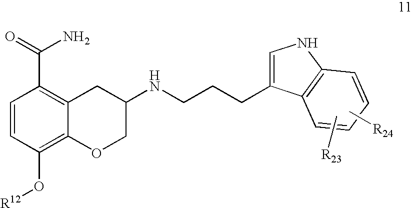
(b) converting the compound of Formula 11 to a compound of Formula 12: 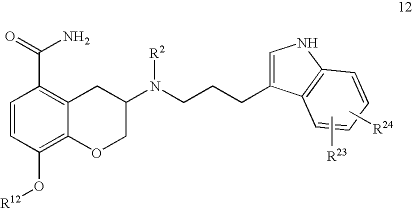
wherein R² is hydrogen, (C₁-C₄)-alkyl, (C₃-C₆)-cycloalkyl, cycloalkylalkyl, aralkyl, or haloalkyl;
under conditions effective to bring about reductive amination at the basic nitrogen of the compound of Formula 11;
(c) optionally reacting the compound of Formula 12 under conditions effective to remove the R¹² group, thereby providing a compound of Formula 13:

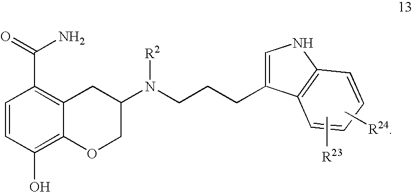

25. The method of claim 24, further comprising:
(a) reacting a compound of Formula 12 or 13 under conditions effective to bring about hydrolysis of the amino group to provide a compound of Formula 16, 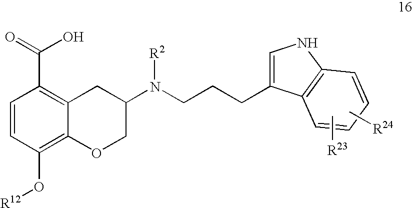
wherein R¹² is hydrogen or (C₁-C₆)-alkyl; and
(b) reacting the compound of Formula 16 with an alkylamine of Formula 16a:
R¹NH₂ (16a)
wherein R¹ is hydrogen or (C₁-C₃)-alkyl; under conditions effective to provide a compound of Formula Ia:

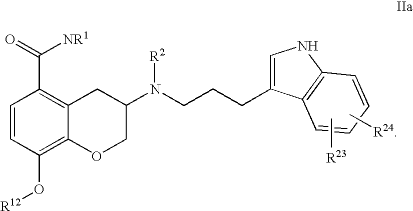

26. The method of claim 25, further comprising reacting the compound of Formula Ia under conditions effective to bring about reduction of the indol to produce a compound of Formula IIIa:

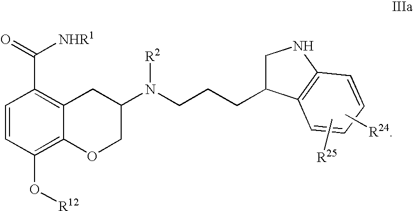

27. A method of synthesizing a compound comprising:
(a) reacting a compound of Formula 10: 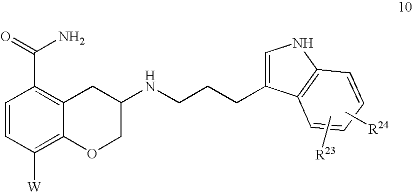
wherein W is halogen:
R²³ and R²⁴ are each independently hydrogen, —F, —Cl, —Br, —I, —CN, or (C₁-C₆)-alkyl;
with an alkyl alcohol salt of Formula 10a:
R¹²O⁻Z⁺ (10a);
wherein R¹² is hydrogen or (C₁-C₆)-alkyl and Z is a pharmaceutically acceptable counter ion;
under conditions effective to produce a compound of Formula 11: 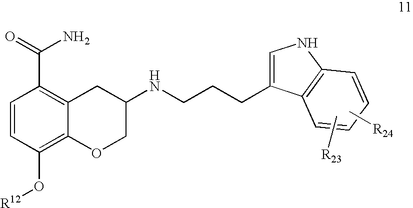
(b) converting the compound of Formula 11 to a compound of Formula 12: 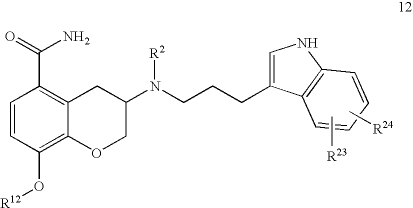
wherein R² is hydrogen, (C₁-C₄)-alkyl, (C₃-C₆)-cycloalkyl, cycloalkylalkyl, aralkyl, or haloalkyl;
under conditions effective to bring about reductive amination at the basic nitrogen of the compound of Formula 11;
(c) reacting the compound of Formula 12 under conditions effective to remove the R¹² group, thereby providing a compound of Formula 13: 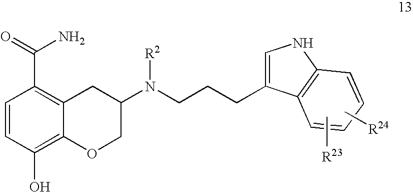
(d) reacting the compound of Formula 13 under conditions effective to produce a compound of Formula 14 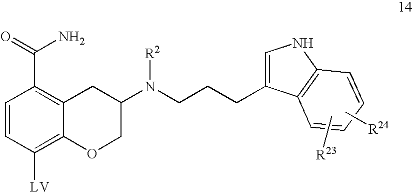
wherein LV is a precursor for a palladium catalyzed reaction; and
(e) reacting a compound of Formula 14 under conditions effective to product a compound of Formula 15: 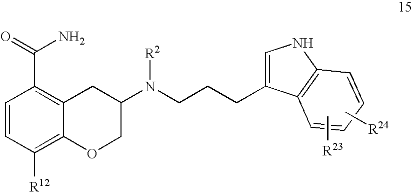
wherein R¹² is (C₁-C₆)-alkyl.

28. The method of claim 27, further comprising
(a) reacting the compound of Formula 15 under conditions effective to bring about hydrolysis of the amino group to provide a compound of Formula 16: 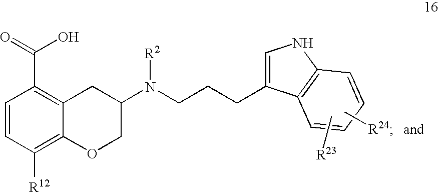
(b) reacting the compound of Formula 16 with an alkylamine of Formula 16a:
R¹NH₂ (16a)
wherein R¹ is hydrogen or (C₁-C₃)-alkyl;
under conditions effective to provide a compound of Formula IIb:

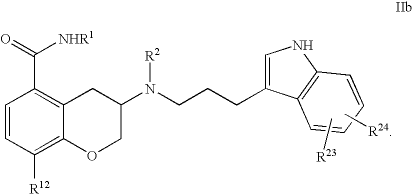

29. The method of claim 28, further comprising reacting the compound of Formula IIb under conditions effective to bring about reduction of the indol to produce a compound of Formula IIIb:

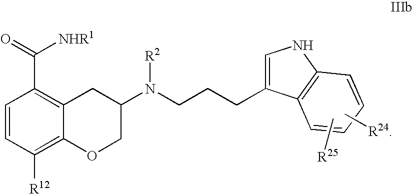

30. A method of synthesizing a compound comprising:
(a) reacting a compound of Formula 10: 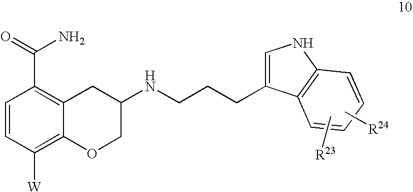
wherein W is halogen:
R²³ and R²⁴ are each independently hydrogen, —F, —Cl, —Br, —I, —CN, or (C₁-C₆)-alkyl;
with an alkylthio salt of Formula 10a or an amine salt of Formula 10b:
R¹²S⁻Z⁺ (10a)
or R¹³R¹⁴N⁻Z⁺ (10b)
wherein R¹², R¹³, and R¹⁴ are each independently hydrogen or (C₁-C₆)-alkyl and Z is a pharmaceutically acceptable counter ion;
under conditions effective to produce a compound of Formula 18: 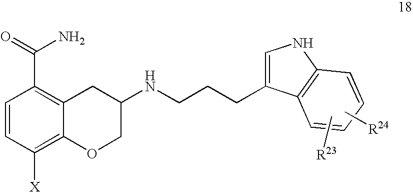
wherein X is —SR¹² or —NR¹³R¹⁴;
(b) converting the compound of Formula 18 to a compound of Formula 15: 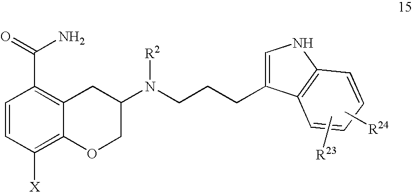
wherein R² is hydrogen, (C₁-C₄)-alkyl, (C₃-C₆)-cycloalkyl, cycloalkylalkyl, aralkyl, or haloalkyl;
under conditions effective to bring about reductive alkylation at the basic nitrogen of Formula 18.

31. The method of claim 30, further comprising:
(a) reacting the compound of Formula 15 under conditions effective to bring about hydrolysis of the amino group to provide a compound of Formula 16: 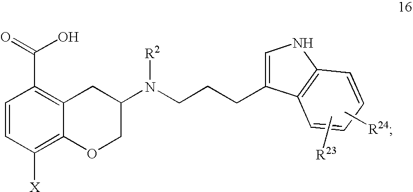
(b) reacting the compound of Formula 16 with an alkylamine of Formula 16a:
R¹NH₂ (16a)
wherein R¹ is hydrogen or (C₁-C₃)-alkyl;
under conditions effective to provide a compound of Formula IIc:

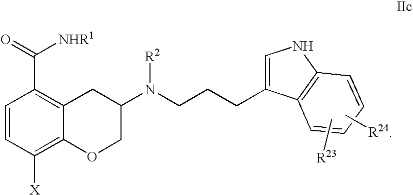

32. The method of claim 31, further comprising reacting the compound of Formula 11c under conditions effective to bring about reduction of the indol to produce a compound of Formula IIIc:

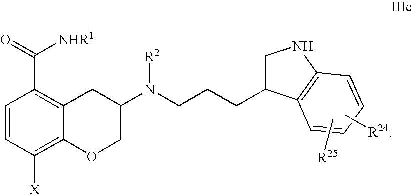

33. The method of claim 24, wherein W is —F, —Cl, or —Br.

34. The method of claim 24, wherein R¹² is (C₁-C₃)-alkyl.

35. The method of claim claims 24, wherein Z is Na⁺, K⁺, or Li⁺.

36. The method of claim 27, wherein LV is triflate.

37. A method of treating a central nervous system disorder in a patient in need thereof, the method comprising administering to the patient an effective amount of a compound of claim 1.

38. The method of claim 37, wherein said central nervous system disorder is an anxiety-related disorder, a cognition-related disorder, depression, or schizophrenia.

39. The method of claim 38, wherein the cognition-related disorder is cognitive deficits, dementia, Parkinson's disease, Huntington's disease, Alzheimer's disease, cognitive deficits associated with Alzheimer's disease, mild cognitive impairment, or schizophrenia.

40. The method of claim 38, wherein the anxiety-related disorder is attention deficit disorder, obsessive compulsive disorder, substance addition, withdrawal from substance addiction, premenstrual dysphoric disorder, social anxiety disorder, anorexia nervosa, or bulimia nervosa.

41. A method of modulating the activity of a 5-HT_{1A} receptor, comprising the step of contacting said receptor with at least one compound of claim 1.

42. A method of binding a 5-HT_{1A} receptor in a patient, comprising the step of administering to the patient an effective amount of at least one compound of claim 1.

43. A method of antagonizing 5-HT_{1A} receptors in a patient, comprising the step of administering to the patient an effective amount of at least one compound of claim 1.

44. A method of agonizing 5-HT_{1A} receptors in a patient, comprising the step of administering to the patient an effective amount of at least one compound of claim 1.

45. A method of modulating serotonin reuptake in a patient, comprising the step of administering to the patient an effective amount of at least one compound of claim 1.

46. A method of treating a patient suspected of suffering from a 5-HT_{1A}-related disorder, comprising the step of administering to the patient a therapeutically effective amount of at least one compound of claim 1.

47. The method according to claim 46, wherein the 5-HT_{1A} disorder is a serotonin disorder.

48. The method according to claim 46, wherein the 5-HT_{1A} disorder is depression, anxiety, panic disorder, post-traumatic stress disorder, premenstrual dysphoric disorder, attention deficit disorder, obsessive compulsive disorder, social anxiety disorder, generalized anxiety disorder, obesity, anorexia nervosa, bulimia nervosa, vasomotor flushing, cocaine addiction, alcohol addiction, or sexual dysfunction.
